SEQUENCE LISTING (118 sequences)

SEQ ID NO: 1 - protein, 6 aa, Mus musculus
RNYWMN

SEQ ID NO: 2 - protein, 6 aa, Mus musculus
TDYAMH

SEQ ID NO: 3 - protein, 6 aa, Artificial Sequence
  Synthetic
  misc_feature (2)..(2): Xaa can be Asn or Asp.
TXYAMN

SEQ ID NO: 4 - protein, 6 aa, Mus musculus
THYSMH

SEQ ID NO: 5 - protein, 6 aa, Mus musculus
SDYFMT

SEQ ID NO: 6 - protein, 6 aa, Mus musculus
TNYDIS

SEQ ID NO: 7 - protein, 6 aa, Mus musculus
SSYWIH

SEQ ID NO: 8 - protein, 19 aa, Artificial Sequence
  Synthetic
  misc_feature (3)..(3): Xaa can be Ser or Thr.
EIXLKSNTYATHYAESVKG

SEQ ID NO: 9 - protein, 17 aa, Mus musculus
WKNTETGESTYAEDFKG

SEQ ID NO: 10 - protein, 17 aa, Artificial Sequence
  Synthetic
  misc_feature (1)..(1): Xaa can be Phe or Trp.
  misc_feature (13)..(14): Xaa in location 13 can be Gln or Asp, Xaa in location 14 can be Asp or Gly.
XINTYTGEPTYAXXFKG

SEQ ID NO: 11 - protein, 17 aa, Mus musculus
WINTETAEPTYVDDFKG

SEQ ID NO: 12 - protein, 17 aa, Mus musculus
NVNYDGRSTYYLDSLKS

SEQ ID NO: 13 - protein, 16 aa, Mus musculus
VIWTGGDTNFNSAFMS

SEQ ID NO: 14 - protein, 17 aa, Mus musculus
LIHPNSGNTDCSETFKN

SEQ ID NO: 15 - protein, 12 aa, Mus musculus
FTKVVADWHLDV

SEQ ID NO: 16 - protein, 11 aa, Mus musculus
GGYGSNYVMDY

SEQ ID NO: 17 - protein, 14 aa, Mus musculus
TRGYYDFDGGAFDY

SEQ ID NO: 18 - protein, 8 aa, Mus musculus
GGLRQGDY

SEQ ID NO: 19 - protein, 12 aa, Mus musculus
EGSQTPLYAVDY

SEQ ID NO: 20 - protein, 13 aa, Mus musculus
VQYFGGSYGPMDY

SEQ ID NO: 21 - protein, 10 aa, Mus musculus
DGASYDWFVH

SEQ ID NO: 22 - protein, 16 aa, Mus musculus
RSSQNIVHSNGNTYLE

SEQ ID NO: 23 - protein, 11 aa, Mus musculus
KASEDIYNRLA

SEQ ID NO: 24 - protein, 11 aa, Artificial Sequence
  Synthetic
  misc_feature (1)..(1): Xaa can be Lys or Arg.
XASQNVGTHLA

SEQ ID NO: 25 - protein, 12 aa, Mus musculus
SATSSVSASYLY

SEQ ID NO: 26 - protein, 11 aa, Mus musculus
KASQNVGTAVA

SEQ ID NO: 27 - protein, 11 aa, Mus musculus
EASDHINDWLA

SEQ ID NO: 28 - protein, 16 aa, Mus musculus
KSSQSLLYTNGKTYLN

SEQ ID NO: 29 - protein, 7 aa, Artificial Sequence
  Synthetic
  misc_feature (2)..(2): Xaa can be Val or Ile.
KXSNRFS

SEQ ID NO: 30 - protein, 7 aa, Mus musculus
GATSLET

SEQ ID NO: 31 - protein, 7 aa, Artificial Sequence
  Synthetic
  misc_feature (3)..(3): Xaa can be Ser or Ile.
SAXYRYI

SEQ ID NO: 32 - protein, 7 aa, Mus musculus
STSNLAS

SEQ ID NO: 33 - protein, 7 aa, Mus musculus
LASNRYT

SEQ ID NO: 34 - protein, 112 aa, Mus musculus
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 35 - protein, 7 aa, Mus musculus
LVSKLDS

SEQ ID NO: 36 - protein, 9 aa, Mus musculus
FQGSHVPFT

SEQ ID NO: 37 - protein, 9 aa, Mus musculus
QQYWNSPRT

SEQ ID NO: 38 - protein, 9 aa, Mus musculus
QQYNTYPLT

SEQ ID NO: 39 - protein, 9 aa, Mus musculus
HQWSSYPYT

SEQ ID NO: 40 - protein, 9 aa, Mus musculus
QQYSIYPFT

SEQ ID NO: 41 - protein, 9 aa, Mus musculus
QQYWNTPLT

SEQ ID NO: 42 - protein, 9 aa, Mus musculus
VQGTHFPRT

SEQ ID NO: 43 - protein, 6 aa, Mus musculus
TNYAMN

SEQ ID NO: 44 - protein, 29 aa, Homo sapiens
QIQLVQSGSELKKPGASVKVSCKASGYTF

SEQ ID NO: 45 - protein, 6 aa, Artificial Sequence
  Synthetic
TDYAMN

SEQ ID NO: 46 - protein, 11 aa, Artificial Sequence
  Synthetic
FDQGTKLEIKR

SEQ ID NO: 47 - protein, 11 aa, Artificial Sequence
  Synthetic
  misc_feature (2)..(2): Xaa can be Gly or Asp.
  misc_feature (11)..(11): Xaa can be Arg or absent.
FXQGTKLEIKX

SEQ ID NO: 48 - protein, 19 aa, Mus musculus
EISLKSNTYATHYAESVKG

SEQ ID NO: 49 - protein, 19 aa, Mus musculus
EITLKSNTYATHYAESVKG

SEQ ID NO: 50 - protein, 17 aa, Mus musculus
FINTYTGEPTYADDFKG

SEQ ID NO: 51 - protein, 17 aa, Mus musculus
WINTYTGEPTYAQGFKG

SEQ ID NO: 52 - protein, 17 aa, Artificial Sequence
  Synthetic
FINTYTGEPTYAQGFKG

SEQ ID NO: 53 - protein, 11 aa, Mus musculus
KASQNVGTHLA

SEQ ID NO: 54 - protein, 11 aa, Mus musculus
RASQNVGTHLA

SEQ ID NO: 55 - protein, 7 aa, Mus musculus
KVSNRFS

SEQ ID NO: 56 - protein, 7 aa, Mus musculus
KISNRFS

SEQ ID NO: 57 - protein, 7 aa, Mus musculus
SASYRYI

SEQ ID NO: 58 - protein, 7 aa, Artificial Sequence
  Synthetic
SAIYRYI

SEQ ID NO: 59 - protein, 123 aa, Mus musculus
EVKLEESGGGLVQPGGSMKLSCVASGFTFRNYWMNWVRQSPERGLEWIAEISLKSNTYATHYAESVKGRFAISRDGSKSSFYLQMNDLRAEDTGIYYCTTFTKVVADWHLDVWGAGTTVTVSS

SEQ ID NO: 60 - protein, 123 aa, Mus musculus
EVKLEESGGGLVQPGGSMILSCVASGFTFRNYWMNWVRQSPERGLEWIAEITLKSNTYATHYAESVKGRFAISRDDSKSSFYLQMNDLRPEDTGIYYCTTFTKVVADWHLDVWGAGTTVTVSS

SEQ ID NO: 61 - protein, 123 aa, Mus musculus
EVRLEESGGGLVQPGGSMKLSCVVSGFTFRNYWMNWVRQSPERGLEWIAEITLKSNTYATHYAESVKGRFAISRDDSKSSFYLQMNDLRPEDTGIYYCTTFTKVVADWHLDVWGAGTTVTVSS

SEQ ID NO: 62 - protein, 120 aa, Mus musculus
QIQLVQSGPELKKPGETVKISCKASGYTFTDYAMHWVKQAPGKGLKWMGWKNTETGESTYAEDFKGRFAFFLETSASTAYLQINNVKNEDTATYFCARGGYGSNYVMDYWGQGTSVIVSS

SEQ ID NO: 63 - protein, 123 aa, Mus musculus
QIQLVQSGPELRKPGETVKISCKASGYSFTNYAMNWVKQAPGKVLKWMGFINTYTGEPTYADDFKGRFAFSLETSASTAYLQINNLKNEDTATYFCTRTRGYYDFDGGAFDYWGQGTSLTVSS

SEQ ID NO: 64 - protein, 123 aa, Artificial Sequence
  Synthetic
QIQLVQSGSELKKPGASVKVSCKASGYTFTNYAMNWVRQAPGQGLEWMGWINTYTGEPTYAQGFKGRFVFSLDTSVSTAYLQISSLKAEDTAVYYCARTRGYYDFDGGAFDYWGQGTLVTVSS

SEQ ID NO: 65 - protein, 123 aa, Artificial Sequence
  Synthetic
QIQLVQSGSELKKPGASVKVSCRARGYTLTDYAMNWVRQAPGQGLEWMGFINTYTGEPTYAQGFKGRFVFSLDTSVSTAYLQIGSLKAEDTAVYYCARTRGYYDFDGGAFDYWGQGTLVTVSS

SEQ ID NO: 66 - protein, 123 aa, Artificial Sequence
  Synthetic
QIQLVQSGSELKKPGASVKVSCRAGGYTLTDYAMNWVRQAPGQGLEWMGFINTYTGEPTYAQGFKGRFVFSLDTSVSTAYLQIGSLKAEDTAVYYCARTRGYYDFDGGAFDYWGQGTLVTVSS

SEQ ID NO: 67 - protein, 123 aa, Artificial Sequence
  Synthetic
QIQLVQSGSELKKPGASVKVSCKASGYSITNYAMNWVRQAPGQGLEWMGFINTYTGEPTYAQGFKGRFVFSLDTSVSTAYLQISSLKAEDTAVYYCARTRGYYDFDGGAFDYWGQGTLVTVSS

SEQ ID NO: 68 - protein, 123 aa, Artificial Sequence
  Synthetic
QIQLVQSGSELKKPGASVKVSCKASGYSITDYAMNWVRQAPGQGLEWMGFINTYTGEPTYAQGFKGRFVFSLDTSVSTAYLQISSLKAEDTAVYYCARTRGYYDFDGGAFDYWGQGTLVTVSS

SEQ ID NO: 69 - protein, 117 aa, Mus musculus
QIQLVQSGPELKKPGETVKISCKASGYTFTHYSMHWVKQAPGKGLKWMGWINTETAEPTYVDDFKGRFAFSLEASASTAFFQINNLKNEDTATYFCARGGLRQGDYWGQGTTLTVSS

SEQ ID NO: 70 - protein, 121 aa, Mus musculus
EVKLVESEGGLVQPGDSMKLSCTASGFTFSDYFMTWIRQVPEKGLEWIANVNYDGRSTYYLDSLKSRFIISRDNANNILYLQMSSLKSEDTATYYCAREGSQTPLYAVDYWGQGTSVTVSS

SEQ ID NO: 71 - protein, 121 aa, Mus musculus
QVQLKESGPGLVAPSESLSITCTVSGFSLTNYDISWIRQSPGKGLEWLGVIWTGGDTNFNSAFMSRLSISKDKSKSQVFLKLNSLQTDDTAIYYCVRVQYFGGSYGPMDYWGQGISVTVSS

SEQ ID NO: 72 - protein, 119 aa, Mus musculus
QVQLQQPRAELVKPGASVMLSCKASGYTFSSYWIHWVRQGPGQGLEWIGLIHPNSGNTDCSETFKNKATLTVDTSSSTAYMQLSSLASEDSAVYYCARDGASYDWFVHWGQGTLVTVSA

SEQ ID NO: 73 - protein, 112 aa, Mus musculus
DVLMTQTPLSLPVSLGDQASISCRSSQNIVHSNGNTYLEWYLQKPGQSPKLLIYKVSNRFSGVPDRFSGSGSGTDFTLRISRVEAEDLGVYYCFQGSHVPFTFGSGTKLEIK

SEQ ID NO: 74 - protein, 112 aa, Mus musculus
DVLMTQTPLSLPVSLGDQASISCRSSQNIVHSNGNTYLEWYLQKPGQSPKLLIYKISNRFSGVPDRFSGSGSGTDFTLRISRVEAEDLGVYYCFQGSHVPFTFGSGTKLEIQ

SEQ ID NO: 75 - protein, 112 aa, Mus musculus
DVLMTQTPLSLPVNLGDQASISCRSSQNIVHSNGNTYLEWYLQKPGQSPKLLIYKVSNRFSGVPDRFSGSGSGTDFTLRISRVEAEDLGVYYCFQGSHVPFTFGSGTKLEIK

SEQ ID NO: 76 - protein, 107 aa, Mus musculus
DIQMTQSSSSFSVSLGDRVTITCKASEDIYNRLAWYQQKPGNAPRLLISGATSLETGVPSRFSGSGSGKDYTLSITSLQTEDVATYYCQQYWNSPRTFGGGTKLEIK

SEQ ID NO: 77 - protein, 107 aa, Mus musculus
DIVMTQSQKFMSTSIGDRVSVTCKASQNVGTHLAWYQQKPGQSPKALIFSASYRYIGVPDRFTGSGSGTDFTLTITNVQSEDLAEYFCQQYNTYPLTFGAGTKLELK

SEQ ID NO: 78 - protein, 107 aa, Artificial Sequence
  Synthetic
DIQMTQSPSSLSASVGDRVTITCRASQNVGTHLAWFQQKPGKAPKSLIYSASYRYIGVPSRFSGSGSGTDFTLTISSLQPEDFATYYCQQYNTYPLTFGQGTKLEIK

SEQ ID NO: 79 - protein, 107 aa, Artificial Sequence
  Synthetic
DIQMTQSPSSLSASVGDRVTITCRASQNVGTHLAWYQQKPGKAPKSLIYSASYRYIGVPSRFSGSGSGTDFTLTISSLQPEDFATYYCQQYNTYPLTFGQGTKLEIK

SEQ ID NO: 80 - protein, 107 aa, Artificial Sequence
  Synthetic
DIQMTQSPSSLSASVGDRVTITCRASQNVGTHLAWYQQKPGKSPKALIFSASYRYIGVPSRFSGSGSGTDFTLTISSLQPEDFATYYCQQYNTYPLTFGQGTKLEIK

SEQ ID NO: 81 - protein, 107 aa, Artificial Sequence
  Synthetic
DIQMTQSPSSLSASVGDRVTITCRASQNVGTHLAWYQQKPGKAPKSLIYSAIYRYIGVPSRFSGSGSGTDFTLTISSLQPEDFATYYCQQYNTYPLTFGQGTKLEIK

SEQ ID NO: 82 - protein, 107 aa, Artificial Sequence
  Synthetic
DIQMTQSPSSLSASVGDRVTITCRASQNVGTHLAWYQQKPGKSPKALIFSAIYRYIGVPSRFSGSGSGTDFTLTISNLQPEDFATYYCQQYNTYPLTFGQGTKLEIK

SEQ ID NO: 83 - protein, 108 aa, Artificial Sequence
  Synthetic
DIQMTQSPSRLGASVGDRVTITCRASQNVGTHLAWYQQKPGKAPKSLIYSAIYRYIGVPSRFSGSGSGTDFTLTISSLQPEDFATYFCQQYNTYPLTFDQGTKLEIKR

SEQ ID NO: 84 - protein, 108 aa, Artificial Sequence
  Synthetic
DIQMTQSPSSLSASVGDRVTITCRASQNVGTHLAWYQQKPGKAPKSLIYSAIYRYIGVPSRFSGSGSGTDFTLTISSLQPEDFATYFCQQYNTYPLTFDQGTKLEIKR

SEQ ID NO: 85 - protein, 108 aa, Mus musculus
QIVLTQSPPIMSASPGEKVTLTCSATSSVSASYLYWFQQKPGSSPKLWIYSTSNLASGVPARFSGSGSGTSYSLTISNMEPADAASYFCHQWSSYPYTFGGGTKLEIK

SEQ ID NO: 86 - protein, 107 aa, Mus musculus
DIVMTQSQKFMSTTVGDRVNITCKASQNVGTAVAWYQQKPGQSPKLLIYLASNRYTGVPDRFTGSGSGTDFTLTVSDMRSEDLADYFCQQYSIYPFTFGSGTKLEIK

SEQ ID NO: 87 - protein, 107 aa, Mus musculus
DIQMTQASSYLSVSLGGRVTITCEASDHINDWLAWYQQTPGNAPRLLISGATSLETGVPSRFSGSGSGKDYTLSITSLQTEDIATYYCQQYWNTPLTFGAGTRLELK

SEQ ID NO: 88 - protein, 112 aa, Mus musculus
DVVMTQTPLTLSVTIGQPASISCKSSQSLLYTNGKTYLNWLLQRPGQSPKRLIYLVSKLDSGVPDRFTGSGSGTDFTLKISRVEAEDLGVYYCVQGTHFPRTFGGGTKLEIK

SEQ ID NO: 89 - protein, 29 aa, Artificial Sequence
  Synthetic
QIQLVQSGSELKKPGASVKVSCRAGGYTL

SEQ ID NO: 90 - protein, 29 aa, Artificial Sequence
  Synthetic
QIQLVQSGSELKKPGASVKVSCRARGYTL

SEQ ID NO: 91 - protein, 29 aa, Artificial Sequence
  Synthetic
QIQLVQSGSELKKPGASVKVSCKASGYSI

SEQ ID NO: 92 - protein, 29 aa, Artificial Sequence
  Synthetic
  misc_feature (2)..(2): Xaa can be Ile or Val.
  misc_feature (23)..(23): Xaa can be Arg or Lys.
  misc_feature (25)..(25): Xaa can be Gly or Arg or Ser.
  misc_feature (28)..(29): Xaa in location 28 can be Thr or Ser, Xaa in location 29 can be Leu or Ile or Phe.
QXQLVQSGSELKKPGASVKVSCXAXGYXX

SEQ ID NO: 93 - protein, 14 aa, Homo sapiens
WVRQAPGQGLEWMG

SEQ ID NO: 94 - protein, 32 aa, Artificial Sequence
  Synthetic
RFVFSLDTSVSTAYLQIGSLKAEDTAVYYCAR

SEQ ID NO: 95 - protein, 32 aa, Homo sapiens
RFVFSLDTSVSTAYLQISSLKAEDTAVYYCAR

SEQ ID NO: 96 - protein, 32 aa, Artificial Sequence
  Synthetic
  misc_feature (18)..(18): Xaa can be Gly or Ser.
RFVFSLDTSVSTAYLQIXSLKAEDTAVYYCAR

SEQ ID NO: 97 - protein, 11 aa, Homo sapiens
WGQGTLVTVSS

SEQ ID NO: 98 - protein, 23 aa, Homo sapiens
DIQMTQSPSSLSASVGDRVTITC

SEQ ID NO: 99 - protein, 23 aa, Artificial Sequence
  Synthetic
DIQMTQSPSRLGASVGDRVTITC

SEQ ID NO: 100 - protein, 23 aa, Artificial Sequence
  Synthetic
  misc_feature (10)..(10): Xaa can be Ser or Arg.
  misc_feature (12)..(12): Xaa can be Ser or Gly.
DIQMTQSPSXLXASVGDRVTITC

SEQ ID NO: 101 - protein, 15 aa, Artificial Sequence
  Synthetic
WYQQKPGKAPKSLIY

SEQ ID NO: 102 - protein, 15 aa, Artificial Sequence
  Synthetic
WYQQKPGKSPKALIF

SEQ ID NO: 103 - protein, 15 aa, Homo sapiens
WFQQKPGKAPKSLIY

SEQ ID NO: 104 - protein, 15 aa, Artificial Sequence
  Synthetic
  misc_feature (2)..(2): Xaa can be Tyr or Phe.
  misc_feature (9)..(9): Xaa can be Ala or Ser.
  misc_feature (12)..(12): Xaa can be Ser or Ala.
  misc_feature (15)..(15): Xaa can be Tyr or Phe.
WXQQKPGKXPKXLIX

SEQ ID NO: 105 - protein, 32 aa, Homo sapiens
GVPSRFSGSGSGTDFTLTISSLQPEDFATYYC

SEQ ID NO: 106 - protein, 32 aa, Artificial Sequence
  Synthetic
GVPSRFSGSGSGTDFTLTISNLQPEDFATYYC

SEQ ID NO: 107 - protein, 32 aa, Artificial Sequence
  Synthetic
GVPSRFSGSGSGTDFTLTISSLQPEDFATYFC

SEQ ID NO: 108 - protein, 32 aa, Artificial Sequence
  Synthetic
  misc_feature (21)..(21): Xaa can be Ser or Asn.
  misc_feature (31)..(31): Xaa can be Tyr or Phe.
GVPSRFSGSGSGTDFTLTISXLQPEDFATYXC

SEQ ID NO: 109 - protein, 10 aa, Homo sapiens
FGQGTKLEIK

SEQ ID NO: 110 - protein, 129 aa, Homo sapiens
EVQLVESGGGLVQPGGSLRLSCAASGFNISYYFIHWVRQAPGKGLEWVASVYSSFGYTYYADSVKGRFTISADTSKNTAYLQMNSLRAEDTAVYYCARFTFPGLFDGFFGAYLGSLDYWGQGTLVTVSS

SEQ ID NO: 111 - protein, 113 aa, Mus musculus
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSS

SEQ ID NO: 112 - protein, 120 aa, Mus musculus
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 113 - protein, 110 aa, Homo sapiens
DIQMTQSPSSLSASVGDRVTITCRASQSVSSAVAWYQQKPGKAPKLLIYSASSLYSGVPSRFSGSRSGTDFTLTISSLQPEDFATYYCQQAVNWVGALVTFGQGTKVEIK

SEQ ID NO: 114 - protein, 108 aa, Mus musculus
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELK

SEQ ID NO: 115 - protein, 118 aa, Homo sapiens
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSS

SEQ ID NO: 116 - protein, 122 aa, Homo sapiens
EVQLLESGGGLVQPGGSLRLSCAASGFTFSSYAMSWVRQAPGKGLEWVATISEYGGSYTYYAESVKGRFTISRDNSKNTLYLQMNSLRAEDTAVYYCARPPYDDYYGGFQYWGQGTLVTVSS

SEQ ID NO: 117 - protein, 108 aa, Homo sapiens
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVL

SEQ ID NO: 118 - protein, 111 aa, Homo sapiens
DIQLTQSPSSLSASVGDRVTITCRASKSVSSGGYSYMHWYQQKPGKAPKLLIYLASNLESGVPSRFSGSGSGTDFTLTISSVQPEDFATYYCQHNRELPVTFGQGTKLEIK

(19) United States
(12) Patent Application Publication
(10) Pub. No.: US 2021/0347908 A1
(43) Pub. Date: Nov. 11, 2021

(54) NOVEL ANTI-SIRPA ANTIBODIES

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Xiaofeng NIU, Shanghai (CN); Jingfeng YU, Shanghai (CN); Jinfeng ZHAO, Shanghai (CN); Fengli WANG, Shanghai (CN); Dan XU, Shanghai (CN); Roumei XING, Shanghai (CN); Zhihao WU, Shanghai (CN); Qinglin DU, Shanghai (CN); Yangsheng QIU, Shanghai (CN); Robert H. ARCH, Shanghai (CN); Hongtao LU, Shanghai (CN)

(21) Appl. No.: 17/283,558

(22) PCT Filed: Aug. 18, 2020
(86) PCT No.: PCT/CN2020/109717

§ 371 Date: Apr. 8, 2021

(30) Foreign Application Priority Data
Aug. 20, 2019 (CN) PCT/CN2019/101564
Aug. 14, 2020 (CN) 202010818127.X

(51) Int. Cl. C07K 16/28; A61K 47/68; A61K 39/395

(52) U.S. Cl. CPC C07K 16/2896; A61K 47/6849; A61K 39/3955; C07K 2317/565; C07K 2317/33; C07K 2317/92; C07K 2317/24; C07K 2317/76; C07K 2317/567

(57) ABSTRACT

Provided are anti-SIRPα antibodies or antigen-binding fragments thereof, isolated polynucleotides encoding the same, pharmaceutical compositions comprising the same and the uses thereof.

### FIELD OF THE INVENTION

The present disclosure generally relates to novel anti-SIRPα antibodies.

### BACKGROUND

Signal-regulatory protein alpha (SIRPα), is an inhibitory receptor expressed primarily on myeloid cells and dendritic cells. In addition to SIRPα, the SIRPs family also includes several other transmembrane glycoproteins, including, SIRPβ and SIRPγ. Each member of the SIRPs family contains 3 similar extracellular Ig-like domains with distinct transmembrane and cytoplasmic domains. CD47 is a broadly expressed transmembrane glycoprotein with an extracellular N-terminal IgV domain, five transmembrane domains, and a short C-terminal intracellular tail. CD47 functions as a cellular ligand for SIRPα. Binding of CD47 to SIRPα delivers a “don't eat me” signal to suppress phagocytosis, and blocking the CD47 mediated engagement of SIRPα on a phagocyte can cause removal of live cells bearing “eat me” signals. Tumor cells frequently overexpress CD47 to evade macrophage-mediated destruction. The interaction of CD47 and SIRPα has been shown to be involved in the regulation of macrophage-mediated phagocytosis (Takenaka et al., Nature Immunol., 8(12): 1313-1323, 2007). In a diverse range of preclinical models, therapies that block the interaction of CD47 and SIRPα stimulate phagocytosis of cancer cells in vitro and anti-tumor immune responses in vivo. Currently, multiple agents targeting CD47 (anti-CD47 antibodies and SIRPα fusion proteins) have proceeded to clinical trials. However, these agents have been associated with hemolytic anemia and thrombocytopenia. In addition to safety issues, universal expression of CD47 may also cause antigen sink, which leads to reduced efficacy.

Needs remain for novel anti-SIRPα antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an antibody or an antigen-binding fragment thereof capable of specifically binding to human SIRPα, comprising a heavy chain variable region comprising HCDR1, HCDR2 and HCDR3, and/or a light chain variable region comprising LCDR1, LCDR2 and LCDR3, wherein a) the HCDR1 comprises a sequence selected from the group consisting of RNYWMN (SEQ ID NO: 1), TDYAMH (SEQ ID NO: 2), TX₁YAMN (SEQ ID NO: 3), THYSMH (SEQ ID NO: 4), SDYFMT (SEQ ID NO: 5), TNYDIS (SEQ ID NO: 6), SSYWIH (SEQ ID NO: 7); and b) the HCDR2 comprises a sequence selected from the group consisting of EIX₂LKSNTYATHYAESVKG (SEQ ID NO: 8), WKNTETGESTYAEDFKG (SEQ ID NO: 9), X₃INTYTGEPTYAX₄X₅FKG (SEQ ID NO: 10), WINTETAEPTYVDDFKG (SEQ ID NO: 11), NVNYDGRSTYYLDSLKS (SEQ ID NO: 12), VIWTGGDTNFNSAFMS (SEQ ID NO: 13), or LIHPNSGNTDCSETFKN (SEQ ID NO: 14); and c) the HCDR3 comprises a sequence selected from the group consisting of FTKVVADWHLDV (SEQ ID NO: 15), GGYGSNYVMDY (SEQ ID NO: 16), TRGYYDFDGGAFDY (SEQ ID NO: 17), GGLRQGDY (SEQ ID NO: 18), EGSQTPLYAVDY (SEQ ID NO: 19), VQYFGGSYGPMDY (SEQ ID NO: 20), DGASYDWFVH (SEQ ID NO: 21); and d) the LCDR1 comprises a sequence selected from the group consisting of RSSQNIVHSNGNTYLE (SEQ ID NO: 22), KASEDIYNRLA (SEQ ID NO: 23), X₆ASQNVGTHLA (SEQ ID NO: 24), SATSSVSASYLY (SEQ ID NO: 25), KASQNVGTAVA (SEQ ID NO: 26), EASDHINDWLA (SEQ ID NO: 27), KSSQSLLYTNGKTYLN (SEQ ID NO: 28); and e) the LCDR2 comprises a sequence selected from the group consisting of KX₇SNRFS (SEQ ID NO: 29), GATSLET (SEQ ID NO: 30), SAX₈YRYI (SEQ ID NO: 31), STSNLAS (SEQ ID NO: 32), LASNRYT (SEQ ID NO: 33), LVSKLDS (SEQ ID NO: 35); and f) the LCDR3 comprises a sequence selected from the group consisting of FQGSHVPFT (SEQ ID NO: 36), QQYWNSPRT (SEQ ID NO: 37), QQYNTYPLT (SEQ ID NO: 38), HQWSSYPYT (SEQ ID NO: 39), QQYSIYPFT (SEQ ID NO: 40), QQYWNTPLT (SEQ ID NO: 41), VQGTHFPRT (SEQ ID NO: 42); wherein X₁ is N or D, X₂ is S or T, X₃ is F or W, X₄ is Q or D, X₅ is D or G, X₆ is K or R, X₇ is V or I, X₈ is S or I.

In some embodiments, the HCDR1 comprises an amino acid sequence of SEQ ID NO: 1, and/or the HCDR2 comprises an amino acid sequence of SEQ ID NO: 8, and/or the HCDR3 comprises an amino acid sequence of SEQ ID NO: 15, and/or the LCDR1 comprises an amino acid sequence of SEQ ID NO: 22, and/or the LCDR2 comprises an amino acid sequence of SEQ ID NO: 29, and/or the LCDR3 comprises an amino acid sequence of SEQ ID NO: 36, wherein X₂ and X₇ are as defined above.

In some embodiments, the HCDR2 comprises an amino acid sequence selected from the group consisting of EISLKSNTYATHYAESVKG (SEQ ID NO: 48), EITLKSNTYATHYAESVKG (SEQ ID NO: 49), and/or the LCDR2 comprises an amino acid sequence selected from the group consisting of KVSNRFS (SEQ ID NO: 55), and KISNRFS (SEQ ID NO: 56).

In some embodiments, the HCDR1 comprises an amino acid sequence of SEQ ID NO: 3, and/or the HCDR2 comprises an amino acid sequence of SEQ ID NO: 10, and/or the HCDR3 comprises an amino acid sequence of SEQ ID NO: 17, and/or the LCDR1 comprises an amino acid sequence of SEQ ID NO: 24, and/or the LCDR2 comprises an amino acid sequence of SEQ ID NO: 31, and/or the LCDR3 comprises an amino acid sequence of SEQ ID NO: 38, wherein X₁, X₃, X₄, X₅, X₆ and X₈ are defined as above.

In some embodiments, the HCDR1 comprises an amino acid sequence selected from the group consisting of TNYAMN (SEQ ID NO: 43) and TDYAMN (SEQ ID NO: 45), and/or the HCDR2 comprises an amino acid sequence selected from the group consisting of FINTYTGEPTYADDFKG (SEQ ID NO: 50), WINTYTGEPTYAQGFKG (SEQ ID NO: 51), and FINTYTGEPTYAQGFKG (SEQ ID NO: 52), and/or the HCDR3 comprises an amino acid sequence of SEQ ID NO: 17, and/or the LCDR1 comprises an amino acid sequence selected from the group consisting of KASQNVGTHLA (SEQ ID NO: 53), and RASQNVGTHLA (SEQ ID NO: 54), and/or the LCDR2 comprises an amino acid sequence selected from the group consisting of SASYRYI (SEQ ID NO: 57) and SAIYRYI (SEQ ID NO: 58), and/or the LCDR3 comprises an amino acid sequence of SEQ ID NO: 38.

In some embodiments, the heavy chain variable region of the antibody or an antigen-binding fragment thereof provided herein comprises a) a HCDR1 comprising the sequence of SEQ ID NO: 1, a HCDR2 comprising the sequence of SEQ ID NO: 48, and a HCDR3 comprising the sequence of SEQ ID NO: 15; or b) a HCDR1 comprising the sequence of SEQ ID NO: 1, a HCDR2 comprising the sequence of SEQ ID NO: 49, and a HCDR3 comprising the sequence of SEQ ID NO: 15; or c) a HCDR1 comprising the sequence of SEQ ID NO: 2, a HCDR2 comprising the sequence of SEQ ID NO: 9, and a HCDR3 comprising the sequence of SEQ ID NO: 16; or d) a HCDR1 comprising the sequence of SEQ ID NO: 43, a HCDR2 comprising the sequence of SEQ ID NO: 50, and a HCDR3 comprising the sequence of SEQ ID NO: 17; or e) a HCDR1 comprising the sequence of SEQ ID NO: 43, a HCDR2 comprising the sequence of SEQ ID NO: 51, and a HCDR3 comprising the sequence of SEQ ID NO: 17; or f) a HCDR1 comprising the sequence of SEQ ID NO: 45, a HCDR2 comprising the sequence of SEQ ID NO: 52, and a HCDR3 comprising the sequence of SEQ ID NO: 17; or g) a HCDR1 comprising the sequence of SEQ ID NO: 43, a HCDR2 comprising the sequence of SEQ ID NO: 52, and a HCDR3 comprising the sequence of SEQ ID NO: 17; or h) a HCDR1 comprising the sequence of SEQ ID NO: 4, a HCDR2 comprising the sequence of SEQ ID NO: 11, and a HCDR3 comprising the sequence of SEQ ID NO: 18; or i) a HCDR1 comprising the sequence of SEQ ID NO: 5, a HCDR2 comprising the sequence of SEQ ID NO: 12, and a HCDR3 comprising the sequence of SEQ ID NO: 19; or j) a HCDR1 comprising the sequence of SEQ ID NO: 6, a HCDR2 comprising the sequence of SEQ ID NO: 13, and a HCDR3 comprising the sequence of SEQ ID NO: 20; or k) a HCDR1 comprising the sequence of SEQ ID NO: 7, a HCDR2 comprising the sequence of SEQ ID NO: 14, and a HCDR3 comprising the sequence of SEQ ID NO: 21.

In some embodiments, the light chain variable region of the antibody or an antigen-binding fragment thereof provided herein comprises a) a LCDR1 comprising the sequence of SEQ ID NO: 22, a LCDR2 comprising the sequence of SEQ ID NO: 55, and a LCDR3 comprising the sequence of SEQ ID NO: 36; or b) a LCDR1 comprising the sequence of SEQ ID NO: 22, a LCDR2 comprising the sequence of SEQ ID NO: 56, and a LCDR3 comprising the sequence of SEQ ID NO: 36; or c) a LCDR1 comprising the sequence of SEQ ID NO: 23, a LCDR2 comprising the sequence of SEQ ID NO: 30, and a LCDR3 comprising the sequence of SEQ ID NO: 37; or d) a LCDR1 comprising the sequence of SEQ ID NO: 53, a LCDR2 comprising the sequence of SEQ ID NO: 57, and a LCDR3 comprising the sequence of SEQ ID NO: 38; or e) a LCDR1 comprising the sequence of SEQ ID NO: 54, a LCDR2 comprising the sequence of SEQ ID NO: 57, and a LCDR3 comprising the sequence of SEQ ID NO: 38; or f) a LCDR1 comprising the sequence of SEQ ID NO: 54, a LCDR2 comprising the sequence of SEQ ID NO: 58, and a LCDR3 comprising the sequence of SEQ ID NO: 38; or g) a LCDR1 comprising the sequence of SEQ ID NO: 25, a LCDR2 comprising the sequence of SEQ ID NO: 32, and a LCDR3 comprising the sequence of SEQ ID NO: 39; or h) a LCDR1 comprising the sequence of SEQ ID NO: 26, a LCDR2 comprising the sequence of SEQ ID NO: 33, and a LCDR3 comprising the sequence of SEQ ID NO: 40; or i) a LCDR1 comprising the sequence of SEQ ID NO: 27, a LCDR2 comprising the sequence of SEQ ID NO: 30, and a LCDR3 comprising the sequence of SEQ ID NO: 41; or j) a LCDR1 comprising the sequence of SEQ ID NO: 28, a LCDR2 comprising the sequence of SEQ ID NO: 35, and a LCDR3 comprising the sequence of SEQ ID NO: 42.

In some embodiments, in the antibody or an antigen-binding fragment thereof provided herein, the HCDR1 comprises the sequence of SEQ ID NO: 1, the HCDR2 comprises the sequence of SEQ ID NO: 48, the HCDR3 comprises the sequence of SEQ ID NO: 15, the LCDR1 comprises the sequence of SEQ ID NO: 22, the LCDR2 comprises the sequence of SEQ ID NO: 55, and the LCDR3 comprises the sequence of SEQ ID NO: 36; or the HCDR1 comprises the sequence of SEQ ID NO: 1, the HCDR2 comprises the sequence of SEQ ID NO: 49, the HCDR3 comprises the sequence of SEQ ID NO: 15, the LCDR1 comprising the sequence of SEQ ID NO: 22, the LCDR2 comprises the sequence of SEQ ID NO: 56, and the LCDR3 comprises the sequence of SEQ ID NO: 36; or the HCDR1 comprises the sequence of SEQ ID NO: 1, the HCDR2 comprises the sequence of SEQ ID NO: 49, the HCDR3 comprises the sequence of SEQ ID NO: 15, the LCDR1 comprises the sequence of SEQ ID NO: 22, the LCDR2 comprises the sequence of SEQ ID NO: 55, and the LCDR3 comprises the sequence of SEQ ID NO: 36; or the HCDR1 comprises the sequence of SEQ ID NO: 2, the HCDR2 comprises the sequence of SEQ ID NO: 9, the HCDR3 comprises the sequence of SEQ ID NO: 16, the LCDR1 comprises the sequence of SEQ ID NO: 23, the LCDR2 comprises the sequence of SEQ ID NO: 30, and the LCDR3 comprises the sequence of SEQ ID NO: 37; or the HCDR1 comprises the sequence of SEQ ID NO: 43, the HCDR2 comprises the sequence of SEQ ID NO: 50, the HCDR3 comprises the sequence of SEQ ID NO: 17, the LCDR1 comprises the sequence of SEQ ID NO: 53, the LCDR2 comprises the sequence of SEQ ID NO: 57, and the LCDR3 comprises the sequence of SEQ ID NO: 38; or the HCDR1 comprises the sequence of SEQ ID NO: 43, the HCDR2 comprises the sequence of SEQ ID NO: 51, the HCDR3 comprises the sequence of SEQ ID NO: 17, the LCDR1 comprises the sequence of SEQ ID NO: 54, the LCDR2 comprises the sequence of SEQ ID NO: 57, and the LCDR3 comprises the sequence of SEQ ID NO: 38; or the HCDR1 comprises the sequence of SEQ ID NO: 45, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 57, and the LCDR3 comprises the sequence of SEQ ID NO: 38; or the HCDR1 comprises the sequence of SEQ ID NO: 45, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 58, and the LCDR3 comprises the sequence of SEQ ID NO: 38; or the HCDR1 comprises the sequence of SEQ ID NO: 43, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 58, and the LCDR3 comprising the sequence of SEQ ID NO: 38; or the HCDR1 comprises the sequence of SEQ ID NO: 4, the HCDR2 comprises the sequence of SEQ ID NO: 11, the HCDR3 comprises the sequence of SEQ ID NO: 18, the LCDR1 comprises the sequence of SEQ ID NO: 25, the LCDR2 comprises the sequence of SEQ ID NO: 32, and the LCDR3 comprises the sequence of SEQ ID NO: 39; or the HCDR1 comprises the sequence of SEQ ID NO: 5, the HCDR2 comprises the sequence of SEQ ID NO: 12, the HCDR3 comprises the sequence of SEQ ID NO: 19, the LCDR1 comprises the sequence of SEQ ID NO: 26, the LCDR2 comprises the sequence of SEQ ID NO: 33, and the LCDR3 comprises the sequence of SEQ ID NO: 40; or the HCDR1 comprises the sequence of SEQ ID NO: 6, the HCDR2 comprises the sequence of SEQ ID NO: 13, the HCDR3 comprises the sequence of SEQ ID NO: 20, the LCDR1 comprises the sequence of SEQ ID NO: 27, the LCDR2 comprises the sequence of SEQ ID NO: 30, and the LCDR3 comprises the sequence of SEQ ID NO: 41; or the HCDR1 comprises the sequence of SEQ ID NO: 7, the HCDR2 comprises the sequence of SEQ ID NO: 14, the HCDR3 comprises the sequence of SEQ ID NO: 21, the LCDR1 comprises the sequence of SEQ ID NO: 28, the LCDR2 comprises the sequence of SEQ ID NO: 35, and the LCDR3 comprises the sequence of SEQ ID NO: 42.

In some embodiments, the antibody or an antigen-binding fragment thereof provided herein further comprises one or more of heavy chain HFR1, HFR2, HFR3 and HFR4, and/or one or more of light chain LFR1, LFR2, LFR3 and LFR4, wherein a) the HFR1 comprises QX₉QLVQSGSELKKPGASVKVSCX₁₀AX₁₁GYX₁₂X₁₃ (SEQ ID NO: 92) or a homologous sequence of at least 80% sequence identity thereof, b) the HFR2 comprises WVRQAPGQGLEWMG (SEQ ID NO: 93) or a homologous sequence of at least 80% sequence identity thereof, c) the HFR3 sequence comprises RFVFSLDTSVSTAYLQIX₁₄SLKAEDTAVYYCAR (SEQ ID NO: 96) or a homologous sequence of at least 80% sequence identity thereof, d) the HFR4 comprises WGQGTLVTVSS (SEQ ID NO: 97) or a homologous sequence of at least 80% sequence identity thereof, e) the LFR1 comprises DIQMTQSPSX₁₅LX₁₆ASVGDRVTITC (SEQ ID NO: 100) or a homologous sequence of at least 80% sequence identity thereof, f) the LFR2 comprises WX₁₇QQKPGKX₁₈PKX₁₉LIX₂₀ (SEQ ID NO: 104) or a homologous sequence of at least 80% sequence identity thereof, g) the LFR3 comprises GVPSRFSGSGSGTDFTLTISX₂₁LQPEDFATYX₂₂C (SEQ ID NO: 108) or a homologous sequence of at least 80% sequence identity thereof, and h) the LFR4 comprises FX₂₃QGTKLEIKX₂₄ (SEQ ID NO: 47) or a homologous sequence of at least 80% sequence identity thereof, wherein X₉ is I or V, X₁₀ is R or K, X₁₁ is G or R or S, X₁₂ is T or S, X₁₃ is L or I or F, X₁₄ is G or S, X₁₅ is S or R, X₁₆ is S or G, X₁₇ is Y or F, X₁₈ is A or S, X₁₉ is S or A, X₂₀ is Y or F, X₂₁ is S or N, X₂₂ is Y or F, X₂₃ is G or D, X₂₄ is R or absent.

In some embodiments, the HFR1 comprises a sequence selected from the group consisting of SEQ ID NOs: 44, 89, 90, and 91, the HFR2 comprises the sequence of SEQ ID NO 93, the HFR3 comprises the sequence selected from the group consisting of SEQ ID NOs: 94 and 95, the HFR4 comprises a sequence of SEQ ID NO: 97, the LFR1 comprises the sequence from the group consisting of SEQ ID NO: 98 and 99, the LFR2 comprises the sequence selected from the group consisting of SEQ ID NOs: 101, 102, and 103, the LFR3 comprises a sequence selected from the group consisting of SEQ ID NOs: 105, 106, and 107 and the LFR4 comprises a sequence selected from the group consisting of SEQ ID NO: 109 and 46.

In some embodiments, the heavy chain variable region of the antibody or an antigen-binding fragment thereof provided herein comprises the sequence selected from the group consisting of SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and a homologous sequence thereof having at least 80% sequence identity yet retaining specific binding affinity to human SIRPα.

In some embodiments, the light chain variable region of the antibody or an antigen-binding fragment thereof provided herein comprises the sequence selected from the group consisting of SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, and a homologous sequence thereof having at least 80% sequence identity yet retaining specific binding affinity to human SIRPα.

In some embodiments, in the antibody or an antigen-binding fragment thereof provided herein, the heavy chain variable region comprises the sequence of SEQ ID NO: 59 and the light chain variable region comprises the sequence of SEQ ID NO: 73; or the heavy chain variable region comprises the sequence of SEQ ID NO: 60 and the light chain variable region comprises the sequence of SEQ ID NO: 74; or the heavy chain variable region comprises the sequence of SEQ ID NO: 61 and the light chain variable region comprises the sequence of SEQ ID NO: 75; or the heavy chain variable region comprises the sequence of SEQ ID NO: 62 and the light chain variable region comprises the sequence of SEQ ID NO: 76; or the heavy chain variable region comprises the sequence of SEQ ID NO: 63 and the light chain variable region comprises the sequence of SEQ ID NO: 77; or the heavy chain variable region comprises the sequence of SEQ ID NO: 64 and the light chain variable region comprises the sequence of SEQ ID NO: 78; or the heavy chain variable region comprises the sequence of SEQ ID NO: 65 and the light chain variable region comprises the sequence of SEQ ID NO: 79; or the heavy chain variable region comprises the sequence of SEQ ID NO: 65 and the light chain variable region comprises the sequence of SEQ ID NO: 80; or the heavy chain variable region comprises the sequence of SEQ ID NO: 66 and the light chain variable region comprises the sequence of SEQ ID NO: 81; or the heavy chain variable region comprises the sequence of SEQ ID NO: 65 and the light chain variable region comprises the sequence of SEQ ID NO: 82; or the heavy chain variable region comprises the sequence of SEQ ID NO: 67 and the light chain variable region comprises the sequence of SEQ ID NO: 83, or the heavy chain variable region comprises the sequence of SEQ ID NO: 68 and the light chain variable region comprises the sequence of SEQ ID NO: 82; or the heavy chain variable region comprises the sequence of SEQ ID NO: 65 and the light chain variable region comprises the sequence of SEQ ID NO: 84; or the heavy chain variable region comprises the sequence of SEQ ID NO: 69 and the light chain variable region comprises the sequence of SEQ ID NO: 85, or the heavy chain variable region comprises the sequence of SEQ ID NO: 70 and the light chain variable region comprises the sequence of SEQ ID NO: 86; or the heavy chain variable region comprises the sequence of SEQ ID NO: 71 and the light chain variable region comprises the sequence of SEQ ID NO: 87; or the heavy chain variable region comprises the sequence of SEQ ID NO: 72 and the light chain variable region comprises the sequence of SEQ ID NO: 88.

In some embodiments, the antibody or an antigen-binding fragment thereof provided herein further comprises one or more amino acid residue substitutions or modifications yet retains specific binding affinity to human SIRPα. In some embodiments, at least one of the substitutions or modifications is in one or more of the CDR sequences, and/or in one or more of the non-CDR sequences of the heavy chain variable region or light chain variable region. In some embodiments, at least one of the substitutions is a conservative substitution.

In some embodiments, the antibody or an antigen-binding fragment thereof provided herein further comprises an Fc region, optionally an Fc region of human immunoglobulin (Ig), or optionally an Fc region of human IgG. In some embodiments, the Fc region is derived from human IgG1, IgG2, IgG3, IgG4, IgA1, IgA2 or IgM. In some embodiments, the Fc region is derived from human IgG4. In some embodiments, the Fc region derived from human IgG4 comprises a S228P mutation. In some embodiments, the Fc region derived from human IgG4 comprises a L235E mutation.

In some embodiments, the antibody or an antigen-binding fragment thereof provided herein is humanized. In some embodiments, the antibody or an antigen-binding fragment thereof provided herein is a monoclonal antibody, a bispecific antibody, a multi-specific antibody, a recombinant antibody, a chimeric antibody, a labeled antibody, a bivalent antibody, an anti-idiotypic antibody or a fusion protein.

In some embodiments, the antibody or an antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or an antigen-binding fragment thereof provided herein has one or more binding properties to human SIRPα selected from the group consisting of: a) having a binding affinity to human SIRPα of no more than 10⁻⁷M as measured by Biacore assay, b) specifically binding to human SIRPα v1 extracellular domain (ECD) at an EC₅₀ of no more than 1 nM as measured by ELISA assay, and c) specifically binding to human SIRPα v2 ECD at an EC₅₀ of no more than 1 nM as measured by ELISA assay.

In some embodiments, the antibody or an antigen-binding fragment thereof provided herein has one or more properties selected from the group consisting of: a) not detectably binding to SIRPγ ECD, b) binding to SIRPγ ECD at an EC₅₀ of no more than 50 nM as measured by ELISA assay; c) specifically binding to SIRPβ ECD at an EC₅₀ of no more than 1 nM as measured by ELISA assay; d) not detectably binding to SIRPβ ECD as measured by ELISA assay; e) specifically binding to human SIRPα IgV domain as measured by FACS binding assay; f) not detectably binding to human SIRPα IgV domain as measured by FACS binding assay; g) specifically binding to mouse SIRPα at a binding affinity of no more than 10⁻⁵M as measured by Biacore assay; h) specifically binding to cyno SIRPα at a concentration of 10 nM as measured by FACS assay; i) capable of inducing phagocytosis of a CD47-expressing target cell by a macrophage cell at a concentration of 10 nM as measured by a phagocytosis assay; and j) not reducing proliferation of CD4⁺ T cells or CD8⁺ T cells.

In another aspect, the prevent disclosure provides an anti-SIRPα antibody or an antigen-binding fragment thereof that competes for binding to human SIRPα with the antibody or an antigen-binding fragment thereof as provided above. In some embodiments, the antibody or an antigen-binding fragment thereof competes for binding to human SIRPα with an antibody comprising a heavy chain variable region comprising the sequence of SEQ ID NO: 70, and a light chain variable region comprising the sequence of SEQ ID NO: 86. In some embodiments, the antibody or an antigen-binding fragment thereof competes for binding to human SIRPα with an antibody comprising a heavy chain variable region comprising the sequence of SEQ ID NO: 72, and a light chain variable region comprising the sequence of SEQ ID NO: 88. In some embodiments, the antibody or an antigen-binding fragment thereof competes for binding to human SIRPα with an antibody comprising a heavy chain variable region comprising the sequence of SEQ ID NO: 62, and a light chain variable region comprising the sequence of SEQ ID NO: 76, or competes for binding to human SIRPα with an antibody comprising a heavy chain variable region comprising the sequence of SEQ ID NO: 69, and a light chain variable region comprising the sequence of SEQ ID NO: 85. In some embodiments, the antibody or an antigen-binding fragment thereof competes for binding to human SIRPα with an antibody comprising a heavy chain variable region comprising the sequence of SEQ ID NO: 71, and a light chain variable region comprising the sequence of SEQ ID NO: 87.

In some embodiments, the antibody or an antigen-binding fragment thereof provided herein is bispecific. In some embodiments, the antibody or an antigen-binding fragment thereof provided herein is capable of specifically binding to a second antigen other than SIRPα, or a second epitope on SIRPα. In some embodiments, the second antigen is selected from the group consisting of CD19, CD20, CD22, CD24, CD25, CD30, CD33, CD38, CD44, CD52, CD56, CD70, CD96, CD97, CD99, CD123, CD279 (PD-1), CD274 (PD-L1), GPC-3, B7-H3, B7-H4, TROP2, CLDN18.2, EGFR, HER2, CD117, C-Met, PTHR2, and HAVCR2 (TIM3).

In some embodiments, the antibody or an antigen-binding fragment thereof provided herein is linked to one or more conjugate moieties. In some embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

In another aspect, the present disclosure provides a pharmaceutical composition comprising the antibody or an antigen-binding fragment thereof of the present disclosure and one or more pharmaceutically acceptable carriers.

In another aspect, the present disclosure provides an isolated polynucleotide encoding the antibody or an antigen-binding fragment thereof of the present disclosure.

In another aspect, the present disclosure provides a vector comprising the isolated polynucleotide of the present disclosure.

In another aspect, the present disclosure provides a host cell comprising the vector of the present disclosure.

In another aspect, the present disclosure provides a kit comprising the antibody or an antigen-binding fragment thereof and/or the pharmaceutical composition of the present disclosure, and a second therapeutic agent.

In another aspect, the present disclosure provides a method of expressing the antibody or an antigen-binding fragment thereof of the present disclosure, comprising culturing the host cell of the present disclosure under the condition at which the vector of the present disclosure is expressed.

In another aspect, the present disclosure provides a method of treating, preventing or alleviating a SIRPα related disease, disorder or condition in a subject, comprising administering to the subject a therapeutically effective amount of the antibody or an antigen-binding fragment thereof of the present disclosure and/or the pharmaceutical composition of the present disclosure. In some embodiments, the disease, disorder or condition is cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis. In some embodiments, the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma. In some embodiments, the cancer is a CD47-positive cancer. In some embodiments, the subject is human. In some embodiments, the administration is via oral, nasal, intravenous, subcutaneous, sublingual, or intramuscular administration. In some embodiments, the method further comprises administering a therapeutically effective amount of a second therapeutic agent. In some embodiments, the second therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

In another aspect, the present disclosure provides a method of modulating SIRPα activity in a SIRPα-positive cell, comprising exposing the SIRPα-positive cell to the antibody or antigen-binding fragment thereof of the present disclosure and/or the pharmaceutical composition of the present disclosure. In some embodiments, the cell is a phagocytic cell.

In another aspect, the present disclosure provides a method of detecting the presence or amount of SIRPα in a sample, comprising contacting the sample with the antibody or an antigen-binding fragment thereof of the present disclosure and/or the pharmaceutical composition of the present disclosure, and determining the presence or the amount of SIRPα in the sample.

In another aspect, the present disclosure provides a method of diagnosing a SIRPα related disease, disorder or condition in a subject, comprising: a) contacting a sample obtained from the subject with the antibody or an antigen-binding fragment thereof of the present disclosure and/or the pharmaceutical composition of the present disclosure; b) determining the presence or amount of SIRPα in the sample; and c) correlating the presence or the amount of SIRPα to existence or status of the SIRPα related disease, disorder or condition in the subject.

In certain embodiments, the antibody or an antigen-binding fragment thereof comprises the HCDR1 comprising the sequence of SEQ ID NO: 5, the HCDR2 comprising the sequence of SEQ ID NO: 12, the HCDR3 comprising the sequence of SEQ ID NO: 19, the LCDR1 comprising the sequence of SEQ ID NO: 26, the LCDR2 comprising the sequence of SEQ ID NO: 33, and the LCDR3 comprising the sequence of SEQ ID NO: 40.

In another aspect, the present disclosure provides use of the antibody or an antigen-binding fragment thereof of the present disclosure and/or the pharmaceutical composition of the present disclosure in the manufacture of a medicament for treating, preventing or alleviating a SIRPα related disease, disorder or condition in a subject.

In another aspect, the present disclosure provides use of the antibody or an antigen-binding fragment thereof of the present disclosure and/or the pharmaceutical composition of the present disclosure in the manufacture of a diagnostic reagent for diagnosing a SIRPα related disease, disorder or condition in a subject. In another aspect, the present disclosure provides a kit comprising the antibody or an antigen-binding fragment thereof of the present disclosure and/or the pharmaceutical composition of the present disclosure, useful in detecting SIRPα. In certain embodiments, the antibody or an antigen-binding fragment thereof comprises the HCDR1 comprising the sequence of SEQ ID NO: 5, the HCDR2 comprising the sequence of SEQ ID NO: 12, the HCDR3 comprising the sequence of SEQ ID NO: 19, the LCDR1 comprising the sequence of SEQ ID NO: 26, the LCDR2 comprising the sequence of SEQ ID NO: 33, and the LCDR3 comprising the sequence of SEQ ID NO: 40.

In another aspect, the present disclosure provides a method of inducing phagocytosis in a subject, comprising administering to the subject the antibody or an antigen-binding fragment thereof of the present disclosure and/or the pharmaceutical composition of the present disclosure in a dose effective to induce phagocytosis. In some embodiments, the subject is human. In some embodiments, the subject has a disease, disorder or condition selected from the group consisting of cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, and arthritis.

In another aspect, the present disclosure provides a method of inducing phagocytosis in vitro, comprising contacting a target cell with a SIRPα positive phagocytic cell sample in the presence of the antibody or an antigen-binding fragment thereof of the present disclosure and/or the pharmaceutical composition of the present disclosure, thereby inducing the phagocytosis of the target cell by the SIRPα positive phagocytic cell. In some embodiments, the target cell is a CD47 expressing cell.

### BRIEF DESCRIPTION OF THE DRAWINGS

FIG. 1 shows ELISA binding specificity of anti-SIRPα antibodies (human IgG4 chimeric antibodies with S228P mutation) against recombinant proteins of human SIRPα v1 ECD (FIG. 1A), human SIRPα v2 ECD (FIG. 1B), human SIRPβ ECD (FIG. 1C) and human SIRPγ ECD (FIG. 1D).

FIG. 2 shows FACS binding curves of anti-SIRPα antibodies (human IgG4 chimeric antibodies with S228P mutation) against CHOK1-human SIRPα v1-1B4 cells (FIG. 2A), CHOK1-cyno SIRPα-2A2 cells (FIG. 2B) and CHOK1-C57BL/6 mouse SIRPα-2.22 cells (FIG. 2C).

FIG. 3 shows phagocytosis of Jurkat cells (FIG. 3A, 3D), Raji cells (FIG. 3B) and DLD-1 cells (FIG. 3C) by human macrophages in the presence of the indicated anti-SIRPα antibodies (human IgG4 chimeric antibodies with S228P mutation).

FIG. 4A illustrates the targeting strategy of B-hSIRPα mice (Biocytogen). FIG. 4B shows binding of anti-SIRPα antibodies (human IgG4 chimeric antibodies with S228P mutation) to B-hSIRPA mice monocytes.

FIG. 5A shows FACS binding curve of humanized antibody hu035.01 against CHOK1-human SIRPα v1-1B4 cells. FIG. 5B shows ELISA binding of humanized antibody hu035.01 against recombinant proteins of human SIRPα v2 ECD and mouse SIRPα (C57BL/6) ECD. FIG. 5C shows full kinetics of binding affinity of humanized antibody hu035.01 against human SIRPα v2 determined by surface plasmon resonance.

FIG. 6 shows ELISA binding specificity of optimized hu035 candidates against recombinant proteins of human SIRPα v1 ECD (FIG. 6A), human SIRPα v2 ECD (FIG. 6B), human SIRPα v8 ECD (FIG. 6C), human SIRPβ ECD (FIG. 6D), human SIRPγ ECD (FIG. 6E) and mouse SIRPα (C57BL/6) ECD (FIG. 6F).

FIG. 7 shows FACS binding curves of optimized hu035 candidates against CHOK1-human SIRPα v1-1B4 cells (FIG. 7A), CHOK1-cyno SIRPα-2A2 cells (FIG. 7B) and CHOK1-C57BL/6 mouse SIRPα-2.22 cells (FIG. 7C).

FIG. 8 shows CD47 and SIRPα interaction blocking activity of optimized hu035 candidates as measured by competitive ELISA assay.

FIG. 9 shows phagocytosis of Jurkat cells (FIG. 9A), DLD1 cells (FIG. 9B), and Raji cells (FIG. 9C) by human macrophages in the presence of chimeric antibody 035c and the optimized hu035 candidates.

FIG. 10 shows CD3/CD28 activator stimulated T cell IFNγ secretion (FIG. 10A), proliferation ratios of CD4⁺ T cells (FIG. 10B) and CD8⁺ T cells (FIG. 10C) in the presence of anti-SIRPα antibodies (human IgG4 chimeric antibodies with S228P mutation) and the optimized hu035 candidates.

FIG. 11 shows allogeneic dendritic cells stimulated T cell IFNγ secretion (FIG. 11A), proliferation ratios of CD4⁺ T cells (FIG. 11B) and CD8⁺ T cells (FIG. 11C) in the presence of anti-SIRPα antibodies (human IgG4 chimeric antibodies with S228P mutation) and the optimized hu035 candidates.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g. of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231 (1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and SIRPα (e.g. human SIRPα).

The ability to “compete for binding to human SIRPα” as used herein refers to the ability of a first antibody or antigen-binding fragment to inhibit the binding interaction between human SIRPα and a second anti-SIRPα antibody to any detectable degree. In certain embodiments, an antibody or antigen-binding fragment that compete for binding to human SIRPα inhibits the binding interaction between human SIRPα and a second anti-SIRPα antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e. including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Name of Amino Acid | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | w |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell, and phagocytosis. Effector functions can be evaluated using various assays such as Fc receptor binding assay, C1q binding assay, and cell lysis assay.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a SIRPα related disease, or refer to identification of a subject with a SIRPα related disease who may benefit from a particular treatment regimen. In some embodiments, diagnosis contains the identification of abnormal amount or activity of SIRPα. In some embodiments, diagnosis refers to the identification of a cancer or an autoimmune disease in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

“SIRPα” as used herein, refers to a regulatory membrane glycoprotein from signal regulatory protein (SIRP) family expressed mainly by myeloid cells, dendritic cells and also by stem cells or neurons. The structure of SIRPα includes an extracellular domain and a cytoplasmic domain. The extracellular domain of SIRPα consists of a membrane-distal Ig variable-like (IgV) fold, and two membrane-proximal Ig constant-like (IgC) folds. The IgV domain of SIRPα is responsible for the binding of the extracellular Ig-domain of CD47. In certain embodiments, the SIRPα is human SIRPα The gene coding for human SIRPα is a polymorphic gene and several variants were described in human population. The most common protein variants are SIRPα v1 and SIRPα v2 (accession numbers NP_542970 (P78324) and CAA71403). SIRPα as used herein may be from other animal species, such as from mouse, and cynomolgus, among others. Exemplary sequence of Mus musculus (mouse) SIRPα protein is disclosed in NCBI Ref Seq No. NP_031573, or BAA20376.1, or BAA13521.1. Exemplary sequence of Cynomolgus (monkey) SIRPα protein is disclosed in NCBI Ref Seq No. NP_001271679.

In addition to SIRPα, the SIRPs family also comprise several other transmembrane glycoproteins, including, SIRPβ and SIRPγ. Each member of the SIRPs family contains 3 similar extracellular Ig-like domains with distinct transmembrane and cytoplasmic domains. “SIRPβ”, encoded by SIRP beta gene, generates a positive signal by intracellular signaling of its cytoplasmic tail through its association with a transmembrane protein called DNAX activation protein 12 or DAP12. The cytoplasmic tail of DAP12 possesses immunoreceptor tyrosine-based activation motifs (ITAMs) that link SIRPβ1 to activation machinery. “SIRPγ”, also named as SIRPg, is encoded by the SIRPG gene, and is highly homologous in the extracellular Ig domains to SIRPα and SIRPβ, but the cytoplasmic tail of SIRPγ is distinct. SIRPγ was also shown to bind to CD47 but with a lower affinity than SIRPα.

The term “anti-SIRPα antibody” refers to an antibody that is capable of specific binding to SIRPα (e.g. human or monkey SIRPα). The term “anti-human SIRPα antibody” refers to an antibody that is capable of specific binding to human SIRPα.

A “SIRPα related” disease, disorder or condition as used herein refers to any disease or condition caused by, exacerbated by, or otherwise linked to increased or decreased expression or activities of SIRPα. In some embodiments, the SIRPα related disease, disorder or condition is an immune-related disorder, such as, for example, an autoimmune disease. In some embodiments, the SIRPα related disease, disorder or condition is a disorder related to excessive cell proliferation, such as, for example, cancer. In certain embodiments, the SIRPα related disease or condition is characterized in expressing or over-expressing of SIRPα gene. In certain embodiments, the SIRPα related disease or condition is characterized in expressing or over-expressing of CD47.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “SIRPα-positive cell” as used herein refer to a cell (e.g. a phagocytic cell) that expresses SIRPα on the surface of the cell. In some embodiments, a “SIRPα-positive cell” may also express SIRPβ or SIRPγ on the surface of the cell.

Anti-SIRPα Antibodies

The present disclosure provides anti-SIRPα antibodies and antigen-binding fragments thereof. The anti-SIRPα antibodies and antigen-binding fragments provided herein are capable of specific binding to SIRPα.

In certain embodiments, the antibodies and the antigen-binding fragments thereof provided herein specifically bind to human SIRPα at an K_{D} value of no more than 10⁻⁷ M, no more than 8×10⁻⁸ M, no more than 5×10⁻⁸ M, no more than 2×10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, or no more than 6×10⁻¹⁰ M by Biacore assay. Biacore assay is based on surface plasmon resonance technology, see, for example, Murphy, M. et al., Current protocols in protein science, Chapter 19, unit 19.14, 2006. In certain embodiments, the K_{D} value is measured by the method as described in Example 4.3 of the present disclosure.

Binding of the antibodies or the antigen-binding fragments thereof provided herein to human SIRPα can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), flow cytometry assay, and other binding assay. In certain embodiments, the antibodies and the antigen-binding fragments thereof provided herein specifically bind to human SIRPα at an EC₅₀ (i.e. 50% binding concentration) of no more than 1 nM, no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.1 nM, no more than 0.09 nM, no more than 0.08 nM, no more than 0.07 nM, no more than 0.06 nM or no more than 0.05 nM by ELISA.

In certain embodiments, the antibodies and the antigen-binding fragments thereof provided herein specifically bind to human SIRPα v1 extracellular domain (ECD) at an EC₅₀ of no more than 1 nM (e.g. no more than 5×10⁻¹⁰ M, no more than 3×10⁻¹⁰ M, no more than 1×10⁻¹⁰ M) as measured by ELISA assay. In certain embodiments, the antibodies and the antigen-binding fragments thereof provided herein specifically bind to human SIRPα v2 ECD at an EC₅₀ of no more than 1 nM (e.g. no more than 5×10⁻¹⁰ M, no more than 3×10⁻¹⁰ M, no more than 1×10⁻¹⁰ M) as measured by ELISA assay.

In certain embodiments, the antibodies and antigen-binding fragments thereof provided herein bind to SIRPγ ECD at an EC₅₀ of no more than 50 nM (e.g. no more than 40 nM, no more than 30 nM, no more than 20 nM, no more than 10 nM, no more than 1 nM) as measured by ELISA assay.

An antibody or antigen-binding fragment thereof that “do not detectably binding” to SIRPγ ECD is one that exhibits no detectable binding to SIRPγ or exhibits a binding to SIRPγ at a level comparable to that a control antibody under equivalent assay conditions. A control antibody can be any antibody that is known not to bind to SIRPγ.

In certain embodiments, the antibodies and antigen-binding fragments thereof provided herein specifically bind to SIRPβ ECD at an EC₅₀ of no more than 1 nM (e.g. no more than 5×10⁻¹⁰ M, no more than 3×10⁻¹⁰ M, no more than 1×10⁻¹⁰ M) as measured by ELISA assay. In certain embodiments, the antibodies and antigen-binding fragments thereof provided herein do not detectably bind to SIRPβ ECD as measured by ELISA assay.

In certain embodiments, the antibodies and antigen-binding fragments thereof provided herein specifically bind to human SIRPα IgV domain as measured by FACS assay. In certain embodiments, the antibodies and antigen-binding fragments thereof provided herein do not detectably bind to human SIRPα IgV domain as measured by FACS assay.

In certain embodiments, the antibodies and antigen-binding fragments thereof provided herein specifically bind to mouse SIRPα at a binding affinity of no more than 10⁻⁵M (e.g. no more than 5×10⁻⁶ M, no more than 3×10⁻⁶ M, no more than 1×10⁻⁶ M, no more than 5×10⁻⁷M, no more than 3×10⁻⁷ M, no more than 1×10⁻⁷ M, no more than 5×10⁻⁸M, no more than 3×10⁻⁸M, no more than 1×10⁻⁸ M) as measured by Biacore assay. In certain embodiments, the antibodies and antigen-binding fragments thereof provided herein specifically bind to cynomoglus SIRPα at a concentration of no more than 10 nM as measured by FACS assay.

In certain embodiments, the antibodies and antigen-binding fragments thereof provided herein are capable of inducing phagocytosis of a CD47-expressing target cell by a macrophage cell at a concentration of no more than 10 nM as measured by a phagocytosis assay.

In certain embodiments, the antibodies and antigen-binding fragments thereof provided herein do not reduce proliferation of CD4⁺ T cells or CD8⁺ T cells. It has been reported that adhesion of human T cells to antigen-presenting cells through SIRPγ-CD47 interaction co-stimulates T cell proliferation. The antibodies and antigen-binding fragments thereof provided herein do not specifically bind to SIRPγ, or do not block SIRPγ-CD47 interaction to such a degree that reduces proliferation of CD4⁺ T cells or CD8⁺ T cells. T cell proliferation can be determined using methods known in the art, for example, by T cell proliferation assay such as those described in Example 5.4 of the present disclosure, for example, by using CellTrace Violet (Life Technologies) labelling to determine proliferation population.

Illustrative Anti-SIRPα Antibodies

In certain embodiments, the present disclosure provides anti-SIRPα antibodies (e.g. anti-human SIRPα antibodies) and antigen-binding fragments thereof comprising one or more (e.g. 1, 2, 3, 4, 5, or 6) CDRs comprising the sequences selected from the group consisting of RNYWMN (SEQ ID NO: 1), TDYAMH (SEQ ID NO: 2), TX₁YAMN (SEQ ID NO: 3), THYSMH (SEQ ID NO: 4), SDYFMT (SEQ ID NO: 5), TNYDIS (SEQ ID NO: 6), SSYWIH (SEQ ID NO: 7), EIX₂LKSNTYATHYAESVKG (SEQ ID NO: 8), WKNTETGESTYAEDFKG (SEQ ID NO: 9), X₃INTYTGEPTYAX₄X₅FKG (SEQ ID NO: 10), WINTETAEPTYVDDFKG (SEQ ID NO: 11), NVNYDGRSTYYLDSLKS (SEQ ID NO: 12), VIWTGGDTNFNSAFMS (SEQ ID NO: 13), or LIHPNSGNTDCSETFKN (SEQ ID NO: 14), FTKVVADWHLDV (SEQ ID NO: 15), GGYGSNYVMDY (SEQ ID NO: 16), TRGYYDFDGGAFDY (SEQ ID NO: 17), GGLRQGDY (SEQ ID NO: 18), EGSQTPLYAVDY (SEQ ID NO: 19), VQYFGGSYGPMDY (SEQ ID NO: 20), DGASYDWFVH (SEQ ID NO: 21), RSSQNIVHSNGNTYLE (SEQ ID NO: 22), KASEDIYNRLA (SEQ ID NO: 23), X₆ASQNVGTHLA (SEQ ID NO: 24), SATSSVSASYLY (SEQ ID NO: 25), KASQNVGTAVA (SEQ ID NO: 26), EASDHINDWLA (SEQ ID NO: 27), KSSQSLLYTNGKTYLN (SEQ ID NO: 28), KX₇SNRFS (SEQ ID NO: 29), GATSLET (SEQ ID NO: 30), SAX₈YRYI (SEQ ID NO: 31), STSNLAS (SEQ ID NO: 32), LASNRYT (SEQ ID NO: 33), LVSKLDS (SEQ ID NO: 35), FQGSHVPFT (SEQ ID NO: 36), QQYWNSPRT (SEQ ID NO: 37), QQYNTYPLT (SEQ ID NO: 38), HQWSSYPYT (SEQ ID NO: 39), QQYSIYPFT (SEQ ID NO: 40), QQYWNTPLT (SEQ ID NO: 41), VQGTHFPRT (SEQ ID NO: 42), wherein X₁ is N or D, X₂ is S or T, X₃ is F or W, X₄ is Q or D, X₅ is D or G, X₆ is K or R, X₇ is V or I, X₈ is S or I. In certain embodiments, the present disclosure further encompass antibodies and antigen binding fragments thereof having no more than one, two or three amino acid residue substitutions to any of SEQ ID NOs: 1-42, wherein X₁ is N or D, X₂ is S or T, X₃ is F or W, X₄ is Q or D, X₅ is D or G, X₆ is K or R, X₇ is V or I, X₈ is S or I.

Antibody “001” as used herein refers to a monoclonal antibody comprising a heavy chain variable region having the sequence of SEQ ID NO: 59, and a light chain variable region having the sequence of SEQ ID NO: 73.

Antibody “002” as used herein refers to a monoclonal antibody comprising a heavy chain variable region having the sequence of SEQ ID NO: 60, and a light chain variable region having the sequence of SEQ ID NO: 74.

Antibody “022” as used herein refers to a monoclonal antibody comprising a heavy chain variable region having the sequence of SEQ ID NO: 62, and a light chain variable region having the sequence of SEQ ID NO: 76.

Antibody “032” as used herein refers to a monoclonal antibody comprising a heavy chain variable region having the sequence of SEQ ID NO: 61, and a light chain variable region having the sequence of SEQ ID NO: 75.

Antibody “035” as used herein refers to a monoclonal antibody comprising a heavy chain variable region having the sequence of SEQ ID NO: 63, and a light chain variable region having the sequence of SEQ ID NO: 77.

Antibody “050” as used herein refers to a monoclonal antibody comprising a heavy chain variable region having the sequence of SEQ ID NO: 69, and a light chain variable region having the sequence of SEQ ID NO: 85.

Antibody “055” as used herein refers to a monoclonal antibody comprising a heavy chain variable region having the sequence of SEQ ID NO: 70, and a light chain variable region having the sequence of SEQ ID NO: 86.

Antibody “060” as used herein refers to a monoclonal antibody comprising a heavy chain variable region having the sequence of SEQ ID NO: 71, and a light chain variable region having the sequence of SEQ ID NO: 87.

Antibody “074” as used herein refers to a monoclonal antibody comprising a heavy chain variable region having the sequence of SEQ ID NO: 72, and a light chain variable region having the sequence of SEQ ID NO: 88.

In certain embodiments, the present disclosure provides anti-SIRPα antibodies and antigen-binding fragments thereof comprising one or more (e.g. 1, 2, 3, 4, 5, or 6) CDRs sequences of Antibody 001, 002, 022, 032, 035, 050, 055, 060, or 074.

In certain embodiments, the present disclosure provides anti-SIRPα antibodies and antigen-binding fragments thereof comprising HCDR1 comprising the sequence selected from the group consisting of SEQ ID NOs: 1-7, HCDR2 comprising the sequence selected from the group consisting of SEQ ID NOs: 8-14, and HCDR3 comprising the sequence selected from the group consisting of SEQ ID NOs: 15-21, and/or LCDR1 comprising the sequence selected from the group consisting of SEQ ID NOs: 22-28, LCDR2 comprising the sequence selected from the group consisting of SEQ ID NOs: 29-33 and 35, and LCDR3 comprising the sequence selected from the group consisting of SEQ ID NOs: 36-42.

In certain embodiments, the present disclosure provides anti-SIRPα antibodies and antigen-binding fragments thereof comprising a HCDR1 comprising the sequence of SEQ ID NO: 1, a HCDR2 comprising the sequence of SEQ ID NO: 48, a HCDR3 comprising the sequence of SEQ ID NO: 15, and/or a LCDR1 comprising the sequence of SEQ ID NO: 22, a LCDR2 comprising the sequence of SEQ ID NO: 55, and a LCDR3 comprising the sequence of SEQ ID NO: 36.

In certain embodiments, the present disclosure provides anti-SIRPα antibodies and antigen-binding fragments thereof comprising a HCDR1 comprising the sequence of SEQ ID NO: 1, a HCDR2 comprising the sequence of SEQ ID NO: 49, a HCDR3 comprising the sequence of SEQ ID NO: 15, and/or a LCDR1 comprising the sequence of SEQ ID NO: 22, a LCDR2 comprising the sequence of SEQ ID NO: 56, and a LCDR3 comprising the sequence of SEQ ID NO: 36.

In certain embodiments, the present disclosure provides anti-SIRPα antibodies and antigen-binding fragments thereof comprising a HCDR1 comprising the sequence of SEQ ID NO: 1, a HCDR2 comprising the sequence of SEQ ID NO: 49, a HCDR3 comprising the sequence of SEQ ID NO: 15, and/or a LCDR1 comprising the sequence of SEQ ID NO: 22, a LCDR2 comprising the sequence of SEQ ID NO: 55, and a LCDR3 comprising the sequence of SEQ ID NO: 36.

In certain embodiments, the present disclosure provides anti-SIRPα antibodies and antigen-binding fragments thereof comprising a HCDR1 comprising the sequence of SEQ ID NO: 2, a HCDR2 comprising the sequence of SEQ ID NO: 9, a HCDR3 comprising the sequence of SEQ ID NO: 16, and/or a LCDR1 comprising the sequence of SEQ ID NO: 23, a LCDR2 comprising the sequence of SEQ ID NO: 30, and a LCDR3 comprising the sequence of SEQ ID NO: 37.

In certain embodiments, the present disclosure provides anti-SIRPα antibodies and antigen-binding fragments thereof comprising a HCDR1 comprising the sequence of SEQ ID NO: 43, a HCDR2 comprising the sequence of SEQ ID NO: 50, a HCDR3 comprising the sequence of SEQ ID NO: 17, and/or a LCDR1 comprising the sequence of SEQ ID NO: 53, a LCDR2 comprising the sequence of SEQ ID NO: 57, and a LCDR3 comprising the sequence of SEQ ID NO: 38.

In certain embodiments, the present disclosure provides anti-SIRPα antibodies and antigen-binding fragments thereof comprising a HCDR1 comprising the sequence of SEQ ID NO: 4, a HCDR2 comprising the sequence of SEQ ID NO: 11, a HCDR3 comprising the sequence of SEQ ID NO: 18, and/or a LCDR1 comprising the sequence of SEQ ID NO: 25, a LCDR2 comprising the sequence of SEQ ID NO: 32, and a LCDR3 comprising the sequence of SEQ ID NO: 39.

In certain embodiments, the present disclosure provides anti-SIRPα antibodies and antigen-binding fragments thereof comprising a HCDR1 comprising the sequence of SEQ ID NO: 5, a HCDR2 comprising the sequence of SEQ ID NO: 12, a HCDR3 comprising the sequence of SEQ ID NO: 19, and/or a LCDR1 comprising the sequence of SEQ ID NO: 26, a LCDR2 comprising the sequence of SEQ ID NO: 33, and a LCDR3 comprising the sequence of SEQ ID NO: 40.

In certain embodiments, the present disclosure provides anti-SIRPα antibodies and antigen-binding fragments thereof comprising a HCDR1 comprising the sequence of SEQ ID NO: 6, a HCDR2 comprising the sequence of SEQ ID NO: 13, a HCDR3 comprising the sequence of SEQ ID NO: 20, and/or a LCDR1 comprising the sequence of SEQ ID NO: 27, a LCDR2 comprising the sequence of SEQ ID NO: 30, and a LCDR3 comprising the sequence of SEQ ID NO: 41.

In certain embodiments, the present disclosure provides anti-SIRPα antibodies and antigen-binding fragments thereof comprising a HCDR1 comprising the sequence of SEQ ID NO: 7, a HCDR2 comprising the sequence of SEQ ID NO: 14, a HCDR3 comprising the sequence of SEQ ID NO: 21, and/or a LCDR1 comprising the sequence of SEQ ID NO: 2, a LCDR2 comprising the sequence of SEQ ID NO: 35, and a LCDR3 comprising the sequence of SEQ ID NO: 42.

Table 1 below shows the CDR amino acid sequences of antibodies 001, 002, 022, 032, 035, 050, 055, 060, and 074. The CDR boundaries were defined or identified by the convention of Kabat. Table 2 below shows the heavy chain and light chain variable region amino acid sequences of antibodies 001, 002, 022, 032, 035, 050, 055, 060, and 074.

TABLE 1
CDR amino acid sequences of 9 antibodies

| Antibody | | CDR1 | CDR2 | CDR3 |
|---|---|---|---|---|
| 001 | HCDR | SEQ ID NO: 1 | SEQ ID NO: 48 | SEQ ID NO: 15 |
| | | RNYWMN | EISLKSNTYATHYA | FTKVVADWHLD |
| | | | ESVKG | V |
| | LCDR | SEQ ID NO: 22 | SEQ ID NO: 55 | SEQ ID NO: 36 |
| | | RSSQNIVHSNG | KVSNRFS | FQGSHVPFT |
| | | NTYLE | | |
| 002 | HCDR | SEQ ID NO: 1 | SEQ ID NO: 49 | SEQ ID NO: 15 |
| | | RNYWMN | EITLKSNTYATHYA | FTKVVADWHLD |
| | | | ESVKG | V |
| | LCDR | SEQ ID NO: 22 | SEQ ID NO: 56 | SEQ ID NO: 36 |
| | | RSSQNIVHSNG | KISNRFS | FQGSHVPFT |
| | | NTYLE | | |
| 032 | HCDR | SEQ ID NO: 1 | SEQ ID NO: 49 | SEQ ID NO: 15 |
| | | RNYWMN | EITLKSNTYATHYA | FTKVVADWHLD |
| | | | ESVKG | V |
| | LCDR | SEQ ID NO: 22 | SEQ ID NO: 55 | SEQ ID NO: 36 |
| | | RSSQNIVHSNG | KVSNRFS | FQGSHVPFT |
| | | NTYLE | | |
| 022 | HCDR | SEQ ID NO: 2 | SEQ ID NO: 9 | SEQ ID NO: 16 |
| | | TDYAMH | WKNTETGESTYAE | GGYGSNYVMDY |
| | | | DFKG | |
| | LCDR | SEQ ID NO: 23 | SEQ ID NO: 30 | SEQ ID NO: 37 |
| | | KASEDIYNRLA | GATSLET | QQYWNSPRT |
| 035 | HCDR | SEQ ID NO: 43 | SEQ ID NO: 50 | SEQ ID NO: 17 |
| | | TNYAMN | FINTYTGEPTYADD | TRGYYDFDGGA |
| | | | FKG | FDY |
| | LCDR | SEQ ID NO: 53 | SEQ ID NO: 57 | SEQ ID NO: 38 |
| | | KASQNVGTHL | SASYRYI | QQYNTYPLT |
| | | A | | |
| 050 | HCDR | SEQ ID NO: 4 | SEQ ID NO: 11 | SEQ ID NO: 18 |
| | | THYSMH | WINTETAEPTYVDD | GGLRQGDY |
| | | | FKG | |
| | LCDR | SEQ ID NO: 25 | SEQ ID NO: 32 | SEQ ID NO: 39 |
| | | SATSSVSASYL | STSNLAS | HQWSSYPYT |
| | | Y | | |
| 055 | HCDR | SEQ ID NO: 5 | SEQ ID NO: 12 | SEQ ID NO: 19 |
| | | SDYFMT | NVNYDGRSTYYLD | EGSQTPLYAVD |
| | | | SLKS | Y |
| | LCDR | SEQ ID NO: 26 | SEQ ID NO: 33 | SEQ ID NO: 40 |
| | | KASQNVGTAV | LASNRYT | QQYSIYPFT |
| | | A | | |
| 060 | HCDR | SEQ ID NO: 6 | SEQ ID NO: 13 | SEQ ID NO: 20 |
| | | TNYDIS | VIWTGGDTNFNSA | VQYFGGSYGPM |
| | | | FMS | DY |
| | LCDR | SEQ ID NO: 27 | SEQ ID NO: 30 | SEQ ID NO: 41 |
| | | EASDHINDWL | GATSLET | QQYWNTPLT |
| | | A | | |
| 074 | HCDR | SEQ ID NO: 7 | SEQ ID NO: 14 | SEQ ID NO: 21 |
| | | SSYWIR | LIFIPNSGNTDCSET | DGASYDWFVH |
| | | | FKN | |
| | LCDR | SEQ ID NO: 28 | SEQ ID NO: 35 | SEQ ID NO: 42 |
| | | KSSQSLLYTNG | LVSKLDS | VQGTHFPRT |
| | | KTYLN | | |

TABLE 2
Variable region amino acid sequences of 9 antibodies

| Antibody | VH | VL |
|---|---|---|
| 001 | SEQ ID NO: 59 | SEQ ID NO: 73 |
| | EVKLEESGGGLVQPGGSMKLS | DVLMTQTPLSLPVSLGDQAS |
| | CVASGFTFRNYWMNWVRQSPE | ISCRSSQNIVHSNGNTYLEW |
| | RGLEWIAEISLKSNTYATHYAES | YLQKPGQSPKLLIYKVSNRF |
| | VKGRFAISRDGSKSSFYLQMND | SGVPDRFSGSGSGTDFTLRIS |
| | LRAEDTGIYYCTTFTKVVADW | RVEAEDLGVYYCFQGSHVPF |
| | FILDVWGAGTTVTVSS | TFGSGTKLEIK |
| 002 | SEQ ID NO: 60 | SEQ ID NO: 74 |
| | EVKLEESGGGLVQPGGSMILSC | DVLMTQTPLSLPVSLGDQAS |
| | VASGFTFRNYWMNWVRQSPER | ISCRSSQNIVHSNGNTYLEW |
| | GLEWIAEITLKSNTYATHYAES | YLQKPGQSPKLLIYKISNRFS |
| | VKGRFAISRDDSKSSFYLQMND | GVPDRFSGSGSGTDFTLRISR |
| | LRPEDTGIYYCTTFTKVVADW | VEAEDLGVYYCFQGSHVPFT |
| | HLDVWGAGTTVTVSS | FGSGTKLEIQ |
| 032 | SEQ ID NO: 61 | SEQ ID NO: 75 |
| | EVRLEESGGGLVQPGGSMKLS | DVLMTQTPLSLPVNLGDQAS |
| | CVVSGFTFRNYWMNWVRQSP | ISCRSSQNIVHSNGNTYLEW |
| | ERGLEWIAEITLKSNTYATHYA | YLQKPGQSPKLLIYKVSNRF |
| | ESVKGRFAISRDDSKSSFYLQM | SGVPDRFSGSGSGTDFTLRIS |
| | NDLRPEDTGIYYCTTFTKVVAD | RVEAEDLGVYYCFQGSHVPF |
| | WHLDVWGAGTTVTVSS | TFGSGTKLEIK |
| 022 | SEQ ID NO: 62 | SEQ ID NO: 76 |
| | QIQLVQSGPELKKPGETVKISC | DIQMTQSSSSFSVSLGDRVTI |
| | KASGYTFTDYAMHWVKQAPG | TCKASEDIYNRLAWYQQKP |
| | KGLKWMGWKNFETGESTYAE | GNAPRLLISGATSLETGVPSR |
| | DFKGRFAFFLETSASTAYLQINN | FSGSGSGKDYTLSITSLQTED |
| | VKNEDTATYFCARGGYGSNYV | VATYYCQQWNSPRTFGGG |
| | MDWGQGTSVIVSS | TKLEIK |
| 035 | SEQ ID NO: 63 | SEQ ID NO: 77 |
| | QIQLVQSGPELRKPGETVKISCK | DIVMTQSQKFMSTSIGDRVS |
| | ASGYSFTNYAMNWVKQAPGK | VTCKASQNVGTHLAWYQQK |
| | VLKWMGFINTYTGEPTYADDF | PGQSPKALIFSASYRYIGVPD |
| | KGRFAFSLETSASTAYLQINNLK | RFTGSGSGTDFTLTITNVQSE |
| | NEDTATYFCTRTRGYYDFDGG | DLAEYFCQQYNTYPLTFGAG |
| | AFDYWGQGTSLTVSS | TKLELK |
| 050 | SEQ ID NO: 69 | SEQ ID NO: 85 |
| | QIQLVQSGPELKKPGETVKISC | QIVLTQSPPIMSASPGEKVTL |
| | KASGYTFTHYSMHWVKQAPG | TCSATSSVSASYLYWFQQKP |
| | KGLKWMGWINTETAEPTYVD | GSSPKLWIYSTSNLASGVPAR |
| | DFKGRFAFSLEASASTAFFQINN | FSGSGSGTSYSLTISNMEPAD |
| | LKNEDTATYFCARGGLRQGDY | AASYFCHQWSSYPYTFGGG |
| | WGQGTTLTVSS | TKLEIK |
| 055 | SEQ ID NO: 70 | SEQ ID NO: 86 |
| | EVKLVESEGGLVQPGDSMKLS | DIVMTQSQKFMSTTVGDRV |
| | CTASGFTFSDYFMTWIRQVPEK | NITCKASQNVGTAVAWYQQ |
| | GLEWIANVNYDGRSTYYLDSL | KPGQSPKLLIYLASNRYTGV |
| | KSRFIISRDNANNILYLQMSSLK | PDRFTGSGSGTDFTLTVSDM |
| | SEDTATYYCAREGSQTPLYAVD | RSEDLADYFCQQYSIYPFTF |
| | YWGQGTSVTVSS | GSGTKLEIK |
| 060 | SEQ ID NO: 71 | SEQ ID NO: 87 |
| | QVQLKESGPGLVAPSESLSITCT | DIQMTQASSYLSVSLGGRVT |
| | VSGFSLTNYDISWIRQSPGKGL | ITCEASDHINDWLAWYQQTP |
| | EWLGVIWTGGDTNFNSAFMSR | GNAPRLLISGATSLETGVPSR |
| | LSISKDKSKSQVFLKLNSLQTD | FSGSGSGKDYTLSITSLQTED |
| | DTAIYYCVRVQYFGGSYGPMD | IATYYCQQYWNTPLTFGAGT |
| | YWGQGISVTVSS | RLELK |
| 074 | SEQ ID NO: 72 | SEQ ID NO: 88 |
| | QVQLQQPRAELVKPGASVMLS | DVVMTQTPLTLSVTIGQPASI |
| | CKASGYTFSSYWIHWVRQGPG | SCKSSQSLLYTNGKTYLNWL |
| | QGLEWIGLIHPNSGNTDCSETF | LQRPGQSPKRLIYLVSKLDSG |
| | KNKATLTVDTSSSTAYMQLSSL | VPDRFTGSGSGTDFTLKISRV |
| | ASEDSAVYYCARDGASYDWFV | EAEDLGVYYCVQGTHFPRTF |
| | HWGQGTLVTVSA | GGGTKLEIK |

Given that each of antibodies 001, 002, 022, 032, 035, 050, 055, 060, and 074 can bind to SIRPα and that antigen-binding specificity is provided primarily by the CDR1, CDR2 and CDR3 regions, the HCDR1, HCDR2 and HCDR3 sequences and LCDR1, LCDR2 and LCDR3 sequences of antibodies 001, 002, 022, 032, 035, 050, 055, 060, and 074 can be “mixed and matched” (i.e., CDRs from different antibodies can be mixed and matched, but each antibody must contain a HCDR1, HCDR2 and HCDR3 and a LCDR1, LCDR2 and LCDR3) to create anti-SIRPα binding molecules of the present disclosure. SIRPα binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence (s). Likewise, when VL CDR sequences are mixed and matched, the LCDR1, LCDR2 and/or LCDR3 sequence from a particular VL sequence preferably is replaced with a structurally similar CDR sequence (s). For example, the HCDR1s of antibodies 001 and 035 share some structural similarity and therefore are amenable to mixing and matching. It will be readily apparent to a person skilled in the art that novel VH and VL sequences can be created by substituting one or more VH and/or VL CDR region sequences with structurally similar sequences from the CDR sequences disclosed herein for monoclonal antibodies 001, 002, 022, 032, 035, 050, 055, 060, and 074.

CDRs are known to be responsible for antigen binding. However, it has been found that not all of the 6 CDRs are indispensable or unchangeable. In other words, it is possible to replace or change or modify one or more CDRs in anti-SIRPα antibodies 001, 002, 022, 032, 035, 050, 055, 060, and 074, yet substantially retain the specific binding affinity to SIRPα.

In certain embodiments, the antibodies and antigen-binding fragments thereof provided herein comprise suitable framework region (FR) sequences, as long as the antibodies and antigen-binding fragments thereof can specifically bind to SIRPα. The CDR sequences provided in Table 1 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Nat. Acad Sci. USA, 89:4285; Presta et al. (1993) J. Immunol., 151:2623).

Table 3 below shows the CDR amino acid sequences of 8 humanized antibodies for antibody 035, which are designated as hu035.01, hu035.02, hu035.03, hu035.09, hu035.10, hu035.13, hu035.14, and hu035.17. The CDR boundaries were defined or identified by the convention of Kabat. Table 4 below shows the heavy chain and light chain variable region amino acid sequences of 8 humanized antibodies hu035.01, hu035.02, hu035.03, hu035.09, hu035.10, hu035.13, hu035.14, and hu035.17. Table 5 below shows the FR amino acid sequences of 8 humanized antibodies hu035.01, hu035.02, hu035.03, hu035.09, hu035.10, hu035.13, hu035.14, and hu035.17.

TABLE 3
CDR amino acid sequences of 8 humanized antibodies

| Antibody | CDR1 | CDR2 | CDR3 | |
|---|---|---|---|---|
| hu035.01 | HCDR | SEQ ID NO: 43 | SEQ ID NO: 51 | SEQ ID NO: 17 |
| | | TNYAMN | WINTYTGEPTYA | TRGYYDFDGGA |
| | | QGFKG | FDY | |
| | LCDR | SEQ ID NO: 54 | SEQ ID NO: 57 | SEQ ID NO: 38 |
| | | RASQNVGTH | SASYRYI | QQYNTYPLT |
| | | LA | | |
| hu035.02 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 57 | SEQ ID NO: 38 |
| | | RASQNVGTH | SASYRYI | QQYNTYPLT |
| | | LA | | |
| hu035.03 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 57 | SEQ ID NO: 38 |
| | | RASQNVGTH | SASYRYI | QQYNTYPLT |
| | | LA | | |
| hu035.09 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 58 | SEQ ID NO: 38 |
| | | RASQNVGTH | SAIYRYI | QQYNTYPLT |
| | | LA | | |
| hu035.10 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 58 | SEQ ID NO: 38 |
| | | RASQNVGTH | SAIYRYI | QQYNTYPLT |
| | | LA | | |
| hu035.13 | HCDR | SEQ ID NO: 43 | SEQ ID NO: 52 | SEQ ID NO: 17 |
| | | TNYAMN | FINTYTGEPTYAQ | TRGYYDFDGGA |
| | | | GFKG | FDY |
| | LCDR | SEQ ID NO: 54 | SEQ ID NO: 58 | SEQ ID NO: 38 |
| | | RASQNVGTH | SAIYRYI | QQYNTYPLT |
| | | LA | | |
| hu035.14 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 58 | SEQ ID NO: 38 |
| | | RASQNVGTH | SAIYRYI | QQYNTYPLT |
| | | LA | | |
| hu035.17 | HCDR | SEQ ID NO: 45 | SEQ ID NO: 52 | SEQ ID NO: 17 |
| | | TDYAMN | FINTYTGEPTYAQ | TRGYYDFDGGA |
| | | | GFKG | FDY |
| | LCDR | SEQ ID NO: 54 | SEQ ID NO: 58 | SEQ ID NO: 38 |
| | | RASQNVGTH | SAIYRYI | QQYNTYPLT |
| | | LA | | |

TABLE 4
Variable region amino acid sequences of 8 humanized antibodies

| Antibody | VH | VL |
|---|---|---|
| hu035.01 | SEQ ID NO: 64 | SEQ ID NO: 78 |
| | QIQLVQSGSELKKPGASVKVS | DIQMTQSPSSLSASVGDRVTI |
| | CKASGYTFTNYAMNWVRQA | TCRASQNVGTHLAWFQQKP |
| | PGQGLEWMGWINTYTGEPTY | GKAPKSLIYSASYRYIGVPSR |
| | AQGFKGRFVFSLDTSVSTAYL | FSGSGSGTDFTLTISSLQPEDF |
| | QISSLKAEDTAVYYCARTRGY | ATYYCQQYNTYPLTFGQGTK |
| | YDFDGGAFDYWGQGTLVTVS | LEIK |
| | S | |
| hu035.02 | SEQ ID NO: 65 | SEQ ID NO: 79 |
| | QIQLVQSGSELKKPGASVKVS | DIQMTQSPSSLSASVGDRVTI |
| | CRARGYTLTDYAMNWVRQA | TCRASQNVGTHLAWYQQKP |
| | PGQGLEWMGFINTYTGEPTY | GKAPKSLIYSASYRYIGVPSR |
| | AQGFKGRFVFSLDTSVSTAYL | FSGSGSGTDFTLTISSLQPEDF |
| | QIGSLKAEDTAVYYCARTRGY | ATYYCQQYNTYPLTFGQGTK |
| | YDFDGGAFDYWGQGTLVTVS | LEIK |
| | S | |
| hu035.03 | SEQ ID NO: 65 | SEQ ID NO: 80 |
| | QIQLVQSGSELKKPGASVKVS | DIQMTQSPSSLSASVGDRVTI |
| | CRARGYTLTDYAMNWVRQA | TCRASQNVGTHLAWYQQKP |
| | PGQGLEWMGFINTYTGEPTY | GKSPKALIFSASYRYIGVPSR |
| | AQGFKGRFVFSLDTSVSTAYL | FSGSGSGTDFTLTISSLQPEDF |
| | QIGSLKAEDTAVYYCARTRGY | ATYYCQQYNTYPLTFGQGTK |
| | YDFDGGAFDYWGQGTLVTVS | LEIK |
| | S | |
| hu035.09 | SEQ ID NO: 66 | SEQ ID NO: 81 |
| | QIQLVQSGSELKKPGASVKVS | DIQMTQSPSSLSASVGDRVTI |
| | CRAGGYTLTDYAMNWVRQA | TCRASQNVGTHLAWYQQKP |
| | PGQGLEWMGFINTYTGEPTY | GKAPKSLIYSAIYRYIGVPSR |
| | AQGFKGRFVFSLDTSVSTAYL | FSGSGSGTDFTLTISSLQPEDF |
| | QIGSLKAEDTAVYYCARTRGY | ATYYCQQYNTYPLTFGQGTK |
| | YDFDGGAFDYWGQGTLVTVS | LEIK |
| | S | |
| hu035.10 | SEQ ID NO: 65 | SEQ ID NO: 82 |
| | QIQLVQSGSELKKPGASVKVS | DIQMTQSPSSLSASVGDRVTI |
| | CRARGYTLTDYAMNWVRQA | TCRASQNVGTHLAWYQQKP |
| | PGQGLEWMGFINTYTGEPTY | GKSPKALIFSAIYRYIGVPSRF |
| | AQGFKGRFVFSLDTSVSTAYL | SGSGSGTDFTLTISNLQPEDF |
| | QIGSLKAEDTAVYYCARTRGY | ATYYCQQYNTYPLTFGQGTK |
| | YDFDGGAFDYWGQGTLVTVS | LEIK |
| | S | |
| hu035.13 | SEQ ID NO: 67 | SEQ ID NO: 83 |
| | QIQLVQSGSELKKPGASVKVS | DIQMTQSPSRLGASVGDRVT |
| | CKASGYSITNYAMNWVRQAP | ITCRASQNVGTHLAWYQQK |
| | GQGLEWMGFINTYTGEPTYA | PGKAPKSLIYSAIYRYIGVPS |
| | QGFKGRFVFSLDTSVSTAYLQI | RFSGSGSGTDFTLTISSLQPE |
| | SSLKAEDTAVYYCARTRGYY | DFATYFCQQYNTYPLTFDQG |
| | DFDGGAFDYWGQGTLVTVSS | TKLEIKR |
| hu035.14 | SEQ ID NO: 68 | SEQ ID NO: 82 |
| | QIQLVQSGSELKKPGASVKVS | DIQMTQSPSSLSASVGDRVTI |
| | CKASGYSITDYAMNWVRQAP | TCRASQNVGTHLAWYQQKP |
| | GQGLEWMGFINTYTGEPTYA | GKSPKALIFSAIYRYIGVPSRF |
| | QGFKGRFVFSLDTSVSTAYLQI | SGSGSGTDFTLTISNLQPEDF |
| | SSLKAEDTAVYYCARTRGYY | ATYYCQQYNTYPLTFGQGTK |
| | DFDGGAFDYWGQGTLVTVSS | LEIK |
| hu035.17 | SEQ ID NO: 65 | SEQ ID NO: 84 |
| | QIQLVQSGSELKKPGASVKVS | DIQMTQSPSSLSASVGDRVTI |
| | CRARGYTLTDYAMNWVRQA | TCRASQNVGTHLAWYQQKP |
| | PGQGLEWMGFINTYTGEPTY | GKAPKSLIYSAIYRYIGVPSR |
| | AQGFKGRFVFSLDTSVSTAYL | FSGSGSGTDFTLTISSLQPEDF |
| | QIGSLKAEDTAVYYCARTRGY | ATYFCQQYNTYPLTFDQGTK |
| | YDFDGGAFDYWGQGTLVTVS | LEIKR |
| | S | |

TABLE 5
FR amino acid sequences of 8 humanized antibodies

| Antibody | | FR1 | FR2 | FR3 | FR4 |
|---|---|---|---|---|---|
| hu035.01 | HFR | SEQ ID NO: 44 | SEQ ID NO: 93 | SEQ ID NO: 95 | SEQ ID NO: 97 |
| | | QIQLVQSGSE | WVRQAPGQG | RFVFSLDTSV | WGQGTLV |
| | | LKKPGASVKV | LEWMG | STAYLQISSLK | TVSS |
| | | SCKASGYTF | | AEDTAVYYC | |
| | | | | AR | |
| | LFR | SEQ ID NO: 98 | SEQ ID NO: 103 | SEQ ID NO: 105 | SEQ ID NO: 109 |
| | | DIQMTQSPSS | WFQQKPGKA | GVPSRFSGSG | FGQGTKLE |
| | | LSASVGDRVT | PKSLIY | SGTDFTLTISS | IK |
| | | ITC | | LQPEDFATYY | |
| | | | | C | |
| hu035.02 | HFR | SEQ ID NO: 90 | SEQ ID NO: 93 | SEQ ID NO: 94 | SEQ ID NO: 97 |
| | | QIQLVQSGSE | WVRQAPGQG | RFVFSLDTSV | WGQGTLV |
| | | LKKPGASVKV | LEWMG | STAYLQIGSL | TVSS |
| | | SCRARGYTL | | KAEDTAVYY | |
| | | | | CAR | |
| | LFR | SEQ ID NO: 98 | SEQ ID NO: 101 | SEQ ID NO: 105 | SEQ ID NO: 109 |
| | | DIQMTQSPSS | WYQQKPGKA | GVPSRFSGSG | FGQGTKLE |
| | | LSASVGDRVT | PKSLIY | SGTDFTLTISS | IK |
| | | ITC | | LQPEDFATYY | |
| | | | | C | |
| hu035.03 | HFR | SEQ ID NO: 90 | SEQ ID NO: 93 | SEQ ID NO: 94 | SEQ ID NO: 97 |
| | | QIQLVQSGSE | WVRQAPGQG | RFVFSLDTSV | WGQGTLV |
| | | LKKPGASVKV | LEWMG | STAYLQIGSL | TVSS |
| | | SCRARGYTL | | KAEDTAVYY | |
| | | | | CAR | |
| | LFR | SEQ ID NO: 98 | SEQ ID NO: 102 | SEQ ID NO: 105 | SEQ ID NO: 109 |
| | | DIQMTQSPSS | WYQQKPGKS | GVPSRFSGSG | FGQGTKLE |
| | | LSASVGDRVT | PKALIF | SGTDFTLTISS | IK |
| | | ITC | | LQPEDFATYY | |
| | | | | C | |
| hu035.09 | HFR | SEQ ID NO: 89 | SEQ ID NO: 93 | SEQ ID NO: 94 | SEQ ID NO: 97 |
| | | QIQLVQSGSE | WVRQAPGQG | RFVFSLDTSV | WGQGTLV |
| | | LKKPGASVKV | LEWMG | STAYLQIGSL | TVSS |
| | | SCRAGGYTL | | KAEDTAVYY | |
| | | | | CAR | |
| | LFR | SEQ ID NO: 98 | SEQ ID NO: 101 | SEQ ID NO: 105 | SEQ ID NO: 109 |
| | | DIQMTQSPSS | WYQQKPGKA | GVPSRFSGSG | FGQGTKLE |
| | | LSASVGDRVT | PKSLIY | SGTDFTLTISS | IK |
| | | ITC | | LQPEDFATYY | |
| | | | | C | |
| hu035.10 | HFR | SEQ ID NO: 90 | SEQ ID NO: 93 | SEQ ID NO: 94 | SEQ ID NO: 97 |
| | | QIQLVQSGSE | WVRQAPGQG | RFVFSLDTSV | WGQGTLV |
| | | LKKPGASVKV | LEWMG | STAYLQIGSL | TVSS |
| | | SCRARGYTL | | KAEDTAVYY | |
| | | | | CAR | |
| | LFR | SEQ ID NO: 98 | SEQ ID NO: 102 | SEQ ID NO: 106 | SEQ ID NO: 109 |
| | | DIQMTQSPSS | WYQQKPGKS | GVPSRFSGSG | FGQGTKLE |
| | | LSASVGDRVT | PKALIF | SGTDFTLTISN | IK |
| | | ITC | | LQPEDFATYY | |
| | | | | C | |
| hu035.13 | HFR | SEQ ID NO: 91 | SEQ ID NO: 93 | SEQ ID NO: 95 | SEQ ID NO: 97 |
| | | QIQLVQSGSE | WVRQAPGQG | RFVFSLDTSV | WGQGTLV |
| | | LKKPGASVKV | LEWMG | STAYLQISSLK | TVSS |
| | | SCKASGYSI | | AEDTAVYYC | |
| | | | | AR | |
| | LFR | SEQ ID NO: 99 | SEQ ID NO: 101 | SEQ ID NO: 107 | SEQ ID NO: 46 |
| | | DIQMTQSPSR | WYQQKPGKA | GVPSRFSGSG | FDQGTKLE |
| | | LGASVGDRVT | PKSLIY | SGTDFTLTISS | IKR |
| | | ITC | | LQPEDFATYF | |
| | | | | C | |
| hu035.14 | HFR | SEQ ID NO: 91 | SEQ ID NO: 93 | SEQ ID NO: 95 | SEQ ID NO: 97 |
| | | QIQLVQSGSE | WVRQAPGQG | RFVFSLDTSV | WGQGTLV |
| | | LKKPGASVKV | LEWMG | STAYLQISSLK | TVSS |
| | | SCKASGYSI | | AEDTAVYYC | |
| | | | | AR | |
| | LFR | SEQ ID NO: 98 | SEQ ID NO: 102 | SEQ ID NO: 106 | SEQ ID NO: 109 |
| | | DIQMTQSPSS | WYQQKPGKS | GVPSRFSGSG | FGQGTKLE |
| | | LSASVGDRVT | PKALIF | SGTDFTLTISN | IK |
| | | ITC | | LQPEDFATYY | |
| | | | | C | |
| hu035.17 | HFR | SEQ ID NO: 90 | SEQ ID NO: 93 | SEQ ID NO: 94 | SEQ ID NO: 97 |
| | | QIQLVQSGSE | WVRQAPGQG | RFVFSLDTSV | WGQGTLV |
| | | LKKPGASVKV | LEWMG | STAYLQIGSL | TVSS |
| | | SCRARGYTL | | KAEDTAVYY | |
| | | | | CAR | |
| | LFR | SEQ ID NO: 98 | SEQ ID NO: 101 | SEQ ID NO: 107 | SEQ ID NO: 46 |
| | | DIQMTQSPSS | WYQQKPGKA | GVPSRFSGSG | FDQGTKLE |
| | | LSASVGDRVT | PKSLIY | SGTDFTLTISS | IKR |
| | | ITC | | LQPEDFATYF | |
| | | | | C | |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-SIRPα antibodies and antigen-binding fragments thereof comprising a heavy chain HFR1 comprising the sequence of QX₉QLVQSGSELKKPGASVKVSCX₁₀AX₁₁GYX₁₂X₁₃ (SEQ ID NO: 92) or a homologous sequence of at least 80% sequence identity thereof, a heavy chain HFR2 comprising the sequence of WVRQAPGQGLEWMG (SEQ ID NO: 93) or a homologous sequence of at least 80% sequence identity thereof, a heavy chain HFR3 comprising the sequence of RFVFSLDTSVSTAYLQIX₁₄SLKAEDTAVYYCAR (SEQ ID NO: 96) or a homologous sequence of at least 80% sequence identity thereof, and a heavy chain HFR4 comprising the sequence of WGQGTLVTVSS (SEQ ID NO: 97) or a homologous sequence of at least 80% sequence identity thereof, wherein X₉ is I or V, X₁₀ is R or K, X₁₁ is G or R or S, X₁₂ is T or S, X₁₃ is L or I or F, X₁₄ is G or S.

In certain embodiments, the present disclosure also provides humanized anti-SIRPα antibodies and antigen-binding fragments thereof comprising a light chain LFR1 comprising the sequence of DIQMTQSPSX₁₅LX₁₆ASVGDRVTITC (SEQ ID NO: 100) or a homologous sequence of at least 80% sequence identity thereof, a light chain LFR2 comprising the sequence of WX₁₇QQKPGKX₁₈PKX₁₉LIX₂₀ (SEQ ID NO: 104) or a homologous sequence of at least 80% sequence identity thereof, a light chain LFR3 comprising the sequence of GVPSRFSGSGSGTDFTLTISX₂₁LQPEDFATYX₂₂C (SEQ ID NO: 108) or a homologous sequence of at least 80% sequence identity thereof, and a light chain LFR4 comprising the sequence of FX₂₃QGTKLEIKX₂₄ (SEQ ID NO: 47) or a homologous sequence of at least 80% sequence identity thereof, wherein X₁₅ is S or R, X₁₆ is S or G, X₁₇ is Y or F, X₁₈ is A or S, X₁₉ is S or A, X₂₀ is Y or F, X₂₁ is S or N, X₂₂ is Y or F, X₂₃ is G or D, X₂₄ is R or absent.

In certain embodiments, the present disclosure also provides humanized anti-SIRPα antibodies and antigen-binding fragments thereof comprising a heavy chain HFR1 comprising a sequence selected from the group consisting of SEQ ID NOs: 44, 89, 90, and 91, a heavy chain HFR2 comprising the sequence of SEQ ID NO 93, a heavy chain HFR3 comprising a sequence selected from the group consisting of SEQ ID NOs: 94 and 95, and a heavy chain HFR4 comprising a sequence of SEQ ID NO: 97; and/or a light chain LFR1 comprising a sequence from the group consisting of SEQ ID NO: 98 and 99, a light chain LFR2 comprising a sequence selected from the group consisting of SEQ ID NOs: 101, 102, and 103, a light chain LFR3 comprising a sequence selected from the group consisting of SEQ ID NOs: 105, 106, and 107, and a light chain LFR4 comprising a sequence selected from the group consisting of SEQ ID NO: 109 and 46.

In certain embodiments, the present disclosure also provides humanized anti-SIRPα antibodies and antigen-binding fragments thereof comprising HFR1, HFR2, HFR3, and/or HFR4 sequences contained in a heavy chain variable region selected from a group consisting of: hu035.01-VH (SEQ ID NO: 64), hu035.02-VH/hu035.03-VH/hu035.10-VH/hu035.17-VH (SEQ ID NO: 65), hu035.09-VH (SEQ ID NO: 66), hu035.13-VH (SEQ ID NO: 67), and hu035.14-VH (SEQ ID NO: 68).

In certain embodiments, the present disclosure also provides humanized anti-SIRPα antibodies and antigen-binding fragments thereof comprising LFR1, LFR2, LFR3, and/or LFR4 sequences contained in a light chain variable region selected from a group consisting of: hu035.01-VL (SEQ ID NO: 78), hu035.02-VL (SEQ ID NO: 79), hu035.03-VL (SEQ ID NO: 80), hu035.09-VL (SEQ ID NO: 81), hu035.10-VL/hu035.14-VL (SEQ ID NO: 82), hu035.13-VL (SEQ ID NO: 83), and hu035.17-VL (SEQ ID NO: 84).

In certain embodiments, the humanized anti-SIRPα antibodies and antigen-binding fragments thereof provided herein comprise a heavy chain variable domain sequence selected from the group consisting of SEQ ID NO: 64, SEQ ID NO: 65, SEQ ID NO: 66, SEQ ID NO: 67, and SEQ ID NO: 68; and/or a light chain variable domain sequence selected from the group consisting of SEQ ID NO: 78, SEQ ID NO: 79, SEQ ID NO: 80, SEQ ID NO: 81, SEQ ID NO: 82, SEQ ID NO: 83, and SEQ ID NO: 84.

The present disclosure also provides exemplary humanized antibodies of 035, including:
1) “hu035.01” comprising the heavy chain variable region of hu035.01-VH (SEQ ID NO: 64) and the light chain variable region of hu035.01-VL (SEQ ID NO: 78);
2) “hu035.02” comprising the heavy chain variable region of hu035.02-VH (SEQ ID NO: 65), and the light chain variable region of hu035.02-VL (SEQ ID NO: 79);
3) “hu035.03” comprising the heavy chain variable region of hu035.03-VH (SEQ ID NO: 65), and the light chain variable region of hu035.03-VL (SEQ ID NO: 80);
4) “hu035.09” comprising the heavy chain variable region of hu035.09-VH (SEQ ID NO: 66), and the light chain variable region of hu035.09-VL (SEQ ID NO: 81);
5) “hu035.10” comprising the heavy chain variable region of hu035.10-VH (SEQ ID NO: 65), and the light chain variable region of hu035.10-VL (SEQ ID NO: 82);
6) “hu035.13” comprising the heavy chain variable region of hu035.13-VH (SEQ ID NO: 67), and the light chain variable region of hu035.13-VL (SEQ ID NO: 83);
7) “hu035.14” comprising the heavy chain variable region of hu035.14-VH (SEQ ID NO: 68), and the light chain variable region of hu035.14-VL (SEQ ID NO: 82);
8) “hu035.17” comprising the heavy chain variable region of hu035.17-VH (SEQ ID NO: 65), and the light chain variable region of hu035.17-VL (SEQ ID NO: 84).

These exemplary humanized anti-SIRPα antibodies retained the specific binding capacity or affinity to SIRPα, and are at least comparable to, or even better than, the parent mouse antibody 035 in that aspect. For example, data is provided in Example 5.

In some embodiments, the anti-SIRPα antibodies and antigen-binding fragments provided herein comprise all or a portion of the heavy chain variable domain and/or all or a portion of the light chain variable domain. In one embodiment, the anti-SIRPα antibody or an antigen-binding fragment thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the anti-SIRPα antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-SIRPα antibodies or the antigen-binding fragments thereof provided herein comprises heavy chain constant regions of human IgG1, IgG2, IgG3, or IgG4. In certain embodiments, the light chain constant region comprises Cκ or Cλ. The constant region of the anti-SIRPα antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-SIRPα antibodies and antigen-binding fragments thereof provided herein comprises an Fc region of IgG1 or IgG3 isotype, which could induce ADCC or CDC; or alternatively, a constant region of IgG4 or IgG2 isotype, which has reduced or depleted effector function. In certain embodiments, the anti-SIRPα antibodies or antigen-binding fragments thereof provided herein comprise a wild type human IgG4 Fc region or other wild type human IgG4 alleles. In certain embodiments, the anti-SIRPα antibodies or antigen-binding fragments thereof provided herein comprise a human IgG4 Fc region comprising a S228P mutation. In certain embodiments, the anti-SIRPα antibodies or antigen-binding fragments thereof provided herein comprise a human IgG4 Fc region comprising a L235E mutation.

In certain embodiments, the antibodies or the antigen-binding fragments thereof provided herein have a specific binding affinity to human SIRPα which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-SIRPα antibody or antigen-binding fragment thereof, which competes for binding to SIRPα with the antibody or antigen-binding fragment thereof provided herein. In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 70, and a light chain variable region comprising the sequence of SEQ ID NO: 86. In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 72, and a light chain variable region comprising the sequence of SEQ ID NO: 88. In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 62, and a light chain variable region comprising the sequence of SEQ ID NO: 76, or competes for binding to human SIRPα with an antibody comprising a heavy chain variable region comprising the sequence of SEQ ID NO: 69, and a light chain variable region comprising the sequence of SEQ ID NO: 85. In certain embodiments, the present disclosure provides an anti-SIRPα antibody or antigen-binding fragment thereof, which competes for binding to human SIRPα with an antibody comprising a heavy chain variable region comprising the sequence of SEQ ID NO: 71, and a light chain variable region comprising the sequence of SEQ ID NO: 87.

In certain embodiments, the present disclosure provides an anti-SIRPα antibody or antigen-binding fragment thereof, which competes for binding to human SIRPα with an antibody selected from the group consisting of: a) an antibody comprising a heavy chain variable region comprising the sequence of SEQ ID NO: 59, and a light chain variable region comprising the sequence of SEQ ID NO: 73; b) an antibody comprising a heavy chain variable region comprising the sequence of SEQ ID NO: 61, and a light chain variable region comprising the sequence of SEQ ID NO: 75; c) an antibody comprising a heavy chain variable region comprising the sequence of SEQ ID NO: 60, and a light chain variable region comprising the sequence of SEQ ID NO: 74; d) an antibody comprising a heavy chain variable region comprising the sequence of SEQ ID NO: 63, and a light chain variable region comprising the sequence of SEQ ID NO: 77, and wherein the antibody or an antigen-binding fragment thereof of is not any of KWAR23, HEFLB, 29-AM4-5, ALX H21 and 3F9-22.

“KWAR23” as used herein refers to an antibody or antigen binding fragment thereof comprising a heavy chain variable region having an amino acid sequence of SEQ ID NO: 111, and a light chain variable region having an amino acid sequence of SEQ ID NO: 114.

“HEFLB” as used herein refers to an antibody or antigen binding fragment thereof comprising a heavy chain variable region having an amino acid sequence of SEQ ID NO: 112, and a light chain variable region having an amino acid sequence of SEQ ID NO: 34.

“29-AM4-5” as used herein refers to an antibody or antigen binding fragment thereof comprising a heavy chain variable region having an amino acid sequence of SEQ ID NO: 110, and a light chain variable region having an amino acid sequence of SEQ ID NO: 113.

“ALX H21” as used herein refers to an antibody or antigen binding fragment thereof comprising a heavy chain variable region having an amino acid sequence of SEQ ID NO: 115, and a light chain variable region having an amino acid sequence of SEQ ID NO: 117.

“3F9-22” as used herein refers to an antibody or antigen binding fragment thereof comprising a heavy chain variable region having an amino acid sequence of SEQ ID NO: 116, and a light chain variable region having an amino acid sequence of SEQ ID NO: 118.

Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences as provided in Tables 1 and 3 above, one or more of the non-CDR sequences of the heavy chain variable region or light chain variable region provided in Tables 2 and 4 above, and/or the constant region (e.g. Fc region). Such variants retain binding specificity to SIRPα of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

Affinity Variants

Affinity variants of antibodies may contain modifications or substitutions in one or more CDR sequences as provided in Tables 1 and 3 above, one or more FR sequences as provided in Table 5 above, or the heavy or light chain variable region sequences provided in Tables 2 and 4 above. FR sequences can be readily identified by a person skilled in the art based on the CDR sequences in Tables 1 and 3 above and variable region sequences in Tables 2 and 4 above, as it is well-known in the art that a CDR region is flanked by two FR regions in the variable region. The affinity variants retain specific binding affinity to SIRPα of the parent antibody, or even have improved SIRPα specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Tables 1 and 3 above, and variable region sequences provided in Tables 2 and 4 above, one or more amino acid residues may be substituted yet the resulting antibody or antigen-binding fragment still retain the binding affinity or binding capacity to SIRPα, or even have an improved binding affinity or capacity. Various methods known in the art can be used to achieve this purpose. For example, a library of antibody variants (such as Fab or scFv variants) can be generated and expressed with phage display technology, and then screened for the binding affinity to human SIRPα. For another example, computer software can be used to virtually simulate the binding of the antibodies to human SIRPα, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-SIRPα antibodies or antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Tables 1 and 3 above yet retaining the specific binding affinity to SIRPα at a level similar to or even higher than its parent antibody.

In certain embodiments, the anti-SIRPα antibodies or antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Tables 2 and 4 above yet retaining the specific binding affinity to SIRPα at a level similar to or even higher than its parent antibody. In some embodiments, a total of 1 to 10 amino acids have been substituted, inserted, or deleted in a variable region sequence listed in Tables 2 and 4 above. In some embodiments, the substitutions, insertions, or deletions occur in regions outside the CDRs (e.g. in the FRs).

Glycosylation Variants

The anti-SIRPα antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

In certain embodiments, the anti-SIRPα antibodies and antigen-binding fragments provided herein comprise a mutation at N297 (e.g. N297A, N297Q, or N297G) to remove the glycosylation site.

Cysteine-Engineered Variants

The anti-SIRPα antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl Methods for engineering antibodies or antigen-binding fragments thereof to introduce free cysteine residues are known in the art, see, for example, WO2006/034488.

Fc Variants

The anti-SIRPα antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821; and WO94/29351 concerning other examples of Fc region variants). One or more amino acids selected from amino acid residues 329, 331 and 322 of the Fc region can be replaced with a different amino acid residue to alter C1q binding and/or reduced or abolished complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-SIRPα antibodies or antigen-binding fragments thereof provided herein has reduced effector functions, and comprise one or more amino acid substitution(s) in IgG1 at a position selected from the group consisting of: 234, 235, 237, and 238, 268, 297, 309, 330, and 331. In certain embodiments, the anti-SIRPα antibodies or antigen-binding fragments thereof provided herein is of IgG1 isotype and comprise one or more amino acid substitution(s) selected from the group consisting of: N297A, N297Q, N297G, L235E, L234A, L235A, L234F, L235E, P331S, and any combination thereof. In certain embodiments, the anti-SIRPα antibodies or antigen-binding fragments thereof provided herein is of IgG2 isotype, and comprises one or more amino acid substitution(s) selected from the group consisting of: H268Q, V309L, A330S, P331S, V234A, G237A, P238S, H268A, and any combination thereof (e.g. H268QN309L/A330S/P331S, V234A/G237A/P238S/H268A/V309L/A330S/P33 S). In certain embodiments, the anti-SIRPα antibodies or antigen-binding fragments thereof provided herein is of IgG4 isotype, and comprises one or more amino acid substitution(s) selected from the group consisting of: N297A, N297Q, N297G, L235E, L234A, L235A, and any combination thereof. In certain embodiments, the anti-SIRPα antibodies or antigen-binding fragments thereof provided herein is of IgG2/IgG4 cross isotype. Examples of IgG2/IgG4 cross isotype is described in Rother R P et al., Nat Biotechnol 25:1256-1264 (2007).

In certain embodiments, the anti-SIRPα antibodies and antigen-binding fragments provided herein is of IgG4 isotype and comprises one or more amino acid substitution(s) at one or more points of 228 and 235. In certain embodiments, the anti-SIRPα antibodies and antigen-binding fragments provided herein is of IgG4 isotype and comprises S228P mutation in the Fc region. In certain embodiments, the anti-SIRPα antibodies and antigen-binding fragments provided herein is of IgG4 isotype and comprises L235E mutation in the Fc region.

In certain embodiments, the anti-SIRPα antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, anti-SIRPα antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

Antigen-Binding Fragments

Provided herein are also anti-SIRPα antigen-binding fragments. Various types of antigen-binding fragments are known in the art and can be developed based on the anti-SIRPα antibodies provided herein, including for example, the exemplary antibodies whose CDRs are shown in Tables 1 and 3 above, and variable sequences are shown in Tables 2 and 4 above, and their different variants (such as affinity variants, glycosylation variants, Fc variants, cysteine-engineered variants and so on).

In certain embodiments, an anti-SIRPα antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. Coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-SIRPα antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

Bispecific Antibodies

In certain embodiments, the anti-SIRPα antibodies or antigen-binding fragments thereof is bispecific. In certain embodiments, the antibody or antigen-binding fragment thereof is further linked to a second functional moiety having a different binding specificity from said SIRPα antibody, or antigen binding fragment thereof.

In certain embodiments, the bispecific antibodies or antigen-binding fragments thereof provided herein are capable of specifically binding to a second antigen other than SIRPα, or a second epitope on SIRPα. In certain embodiments, the second antigen is selected from the group consisting of CD19, CD20, CD22, CD24, CD25, CD30, CD33, CD38, CD44, CD52, CD56, CD70, CD96, CD97, CD99, CD123, CD279 (PD-1), CD274 (PD-L1), GPC-3, B7-H3, B7-H4, TROP2, CLDN18.2, EGFR, HER2, CD117, C-Met, PTHR2, and HAVCR2 (TIM3).

Conjugates

In some embodiments, the anti-SIRPα antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other anticancer drugs.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (I) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moieties, digoxigenin, biotin/avidin, DNA molecules or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibodies or antigen-binding fragments thereof provided herein is used as a base for a conjugate.

Polynucleotides and Recombinant Methods

The present disclosure provides isolated polynucleotides that encode the anti-SIRPα antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-SIRPα antibodies or antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

The present disclosure provides vectors comprising the isolated polynucleotide provided herein. In certain embodiments, the polynucleotide provided herein encodes the antibodies or antigen-binding fragments thereof, at least one promoter (e.g. SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-SIRPα antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neuraspora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for anti-SIRPα antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866, 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-SIRPα antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

Pharmaceutical Composition

The present disclosure further provides pharmaceutical compositions comprising the anti-SIRPα antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-SIRPα antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

Kits

In certain embodiments, the present disclosure provides a kit comprising the antibody or an antigen-binding fragment thereof provided herein. In certain embodiments, the present disclosure provides a kit comprising the antibody or an antigen-binding fragment thereof provided herein, and a second therapeutic agent. In certain embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

Methods of Use

The present disclosure also provides methods of treating a SIRPα related disease, disorder or condition in a subject, comprising administering to the subject a therapeutically effective amount of the antibody or antigen-binding fragment thereof provided herein, and/or the pharmaceutical composition provided herein. In certain embodiments, the subject is human.

In some embodiments, the SIRPα related disease, disorder or condition is characterized in expressing or over-expressing of SIRPα and/or SIRPα signature genes.

In certain embodiments, the SIRPα related disease, disorder or condition include, but are not limited to, cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis.

In certain embodiments, the cancer is a SIRPα-expressing cancer. In certain embodiments, the cancer is a CD47-positive cancer. In certain embodiments, the cancer is selected from the group consisting of anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma.

In some embodiments, the cancer is a CD47-positive cancer. In some embodiments, the subject to be treated has been identified as having a CD47-positive cancer. “CD47-positive” cancer as used herein refers to a cancer characterized in expressing CD47 protein in a cancer cell, or expressing CD47 in a cancer cell at a level significantly higher than that would have been expected of a normal cell. The presence and/or amount of CD47 in an interested biological sample can be indicative of whether the subject from whom the biological sample is derived could likely respond to an anti-SIRPα antibody. Various methods can be used to determine the presence and/or amount of CD47 in a test biological sample from the subject. For example, the test biological sample can be exposed to anti-CD47 antibody or antigen-binding fragment thereof, which binds to and detects the expressed CD47 protein. Alternatively, CD47 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. In certain embodiments, presence or up-regulated level of the CD47 in the test biological sample indicates likelihood of responsiveness. The term “up-regulated” as used herein, refers to an overall increase of no less than 10%, 15%, 20%, 25%, 30%, 35%, 40/s, 45%, 50%, 55%, 60%, 65%, 70%, 75%, 80% or greater, in the expression level of CD47 in the test sample, as compared to the CD47 expression level in a reference sample as detected using the same method. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In another aspect, methods are provided to treat a disease, disorder or condition in a subject that would benefit from modulation of SIRPα activity, comprising administering a therapeutically effective amount of the antibody or antigen-binding fragment thereof provided herein and/or the pharmaceutical composition provided herein to a subject in need thereof. In certain embodiments, the disease or condition is a SIRPα related disease, disorder or condition.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The antibodies or antigen-binding fragments thereof provided herein may be administered by any route known in the art, such as for example parenteral (e.g. subcutaneous, intraperitoneal, intravenous, including intravenous infusion, intramuscular, or intradermal injection) or non-parenteral (e.g. oral, intranasal, intraocular, sublingual, rectal, or topical) routes.

In some embodiments, the antibodies or antigen-binding fragments thereof provided herein may be administered alone or in combination a therapeutically effective amount of a second therapeutic agent. For example, the antibodies or antigen-binding fragments thereof disclosed herein may be administered in combination with a second therapeutic agent, for example, a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, or cytokines.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In another aspect, the present disclosure further provides methods of modulating SIRPα activity in SIRPα-positive cells, comprising exposing the SIRPα-positive cells to the antibodies or antigen-binding fragments thereof provided herein. In some embodiments, the SIRPα-positive cell is a phagocytic cell (e.g. a macrophage).

In another aspect, the present disclosure provides methods of detecting the presence or amount of SIRPα in a sample, comprising contacting the sample with the antibody or antigen-binding fragment thereof provided herein, and determining the presence or the amount of SIRPα in the sample.

In another aspect, the present disclosure provides a method of diagnosing a SIRPα related disease, disorder or condition in a subject, comprising: a) contacting a sample obtained from the subject with the antibody or an antigen-binding fragment thereof provided herein; b) determining the presence or amount of SIRPα in the sample; and c) correlating the presence or the amount of SIRPα to existence or status of the SIRPα related disease, disorder or condition in the subject.

In another aspect, the present disclosure provides kits comprising the antibody or antigen-binding fragment thereof provided herein, optionally conjugated with a detectable moiety, which is useful in detecting a SIRPα related disease, disorder or condition. The kits may further comprise instructions for use.

In another aspect, the present disclosure also provides use of the antibody or antigen-binding fragment thereof provided herein in the manufacture of a medicament for treating, preventing or alleviating a SIRPα related disease, disorder or condition in a subject, in the manufacture of a diagnostic reagent for diagnosing a SIRPα related disease, disorder or condition.

In another aspect, the present disclosure provides a method of inducing phagocytosis in a subject, comprising administering to the subject the antibody or an antigen-binding fragment thereof provided herein and/or the pharmaceutical composition provided herein in a dose effective to induce phagocytosis. For example, the antibody or an antigen-binding fragment thereof provided herein may be administered to induce phagocytosis of cancer cells, inflammatory cells, and/or chronically infected cells that express CD47. In some embodiments, the subject is human. In some embodiments, the subject has a disease, disorder or condition selected from the group consisting of cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, and arthritis.

In another aspect, the present disclosure provides a method of inducing phagocytosis in vitro, comprising contacting a target cell with a SIRPα positive phagocytic cell sample in the presence of the antibody or an antigen-binding fragment thereof provided herein, thereby inducing the phagocytosis of the target cell by the SIRPα positive phagocytic cell. In some embodiments, the target cell is a CD47 expressing cell.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1. Reagent Generation

1.1 Reference Antibody Generation

The DNA sequences encoding variable regions of anti-SIRPα reference antibodies 29-AM4-5 (see US20140242095), KWAR23 (see US20170073414A1), HEFLB (see WO2017178653A2), ALX H21 (see US20180105600A1) or 3F9-22 (see US20190359707A1) were cloned into the vectors expressing human IgG constant regions. The variable region amino acid sequences of reference antibodies 29-AM4-5, KWAR23, HEFLB, ALX H21 and 3F9-22 are shown in Table 6 below. The expression plasmids transfected Expi293 cells (Invitrogen) were cultured at 37° C. for a week. Then the culture medium was collected and centrifuged to remove cell pellets. The harvested supernatant was purified using Protein A affinity chromatography column (Mabselect Sure, GE Healthcare).

TABLE 6
Variable region amino acid sequences of 5 reference antibodies

| Antibody | VH | VL |
|---|---|---|
| 29-AM4-5 | SEQ ID NO: 110 | SEQ ID NO: 113 |
| | EVQLVESGGGLVQPGGSLRLSC | DIQMTQSPSSLSASVGDRVTI |
| | AASGFNISYYFIHWVRQAPGKG | TCRASQSVSSAVAWYQQKPG |
| | LEWVASVYSSFGYTYYADSVK | KAPKLLIYSASSLYSGVPSRF |
| | GRFTISADTSKNTAYLQMNSLR | SGSRSGTDFTLTISSLQPEDFA |
| | AEDTAVYYCARFTFPGLFDGFF | TYYCQQAVNWVGALVTFGQ |
| | GAYLGSLDYWGQGTLVTVSS | GTKVEIK |
| KWAR23 | SEQ ID NO: 111 | SEQ ID NO: 114 |
| | EVQLQQSGAELVKPGASVKLS | QIVLTQSPAIMSASPGEKVTL |
| | CTASGFNIKDYYITIWVQQRTEQ | TCSASSSVSSSYLYWYQQKP |
| | GLEWIGRIDPEDGETKYAPKFQ | GSSPKLWIYSTSNLASGVPAR |
| | DKATITADTSSNTAYLHLSSLTS | FSGSGSGTSYSLTISSMEAED |
| | EDTAVYYCARWGAWGQGTL | AASYFCHQWSSYPRTFGAGT |
| | VTVSS | KLELK |
| HEFLB | SEQ ID NO: 112 | SEQ ID NO: 34 |
| | EVQLVQSGAEVKKPGESLRISC | DVVMTQSPLSLPVTLGQPASI |
| | KASGYSFTSYWVHWVRQMPG | SCRSSQSLVHSYGNTYLYWF |
| | KGLEWMGNIDPSDSDTHYSPSF | QQRPGQSPRLLIYRVSNRFSG |
| | QGHVTLSVDKSISTAYLQLSSL | VPDRFSGSGSGTDFTLKISRV |
| | KASDTAMYYCVRGGTGTLAYF | EAEDVGVYYCFQGTHVPYT |
| | AYWGQGTLVTVSS | FGGGTKVEIK |
| ALX H21 | SEQ ID NO: 115 | SEQ ID NO: 117 |
| | EVQLVESGGGVVQPGGSLRLS | SYELTQPPSVSVSPGQTARIT |
| | CAASGFTFSSNAMSWVRQAPG | CSGGSYSSYYYAWYQQKPG |
| | KGLEWVAGISAGGSDTYYPAS | QAPVTLIYSDDKRPSNIPERF |
| | VKGRFTISRDNSKNTLYLQMNS | SGSSSGTTVTLTISGVQAEDE |
| | LRAEDTAVYYCARETWNHLFD | ADYYCGGYDQSSYTNPFGG |
| | YWGQGTLVTVSS | GTKLTVL |
| 3F9-22 | SEQ ID NO: 116 | SEQ ID NO: 118 |
| | EVQLLESGGGLVQPGGSLRLSC | DIQLTQSPSSLSASVGDRVTI |
| | AASGFTFSSYAMSWVRQAPGK | TCRASKSVSSGGYSYMHWY |
| | GLEWVATISEYGGSYTYYAESV | QQKPGKAPKLLIYLASNLES |
| | KGRFTISRDNSKNTLYLQMNSL | GYPSRFSGSGSGTDFTLTISS |
| | RAEDTAVYYCARPPYDDYYGG | VQPEDFATYYCQHNRELPVT |
| | FQYWGQGTLVTVSS | FGQGTKLEIK |

1.2. Human, Cynomolgus Monkey, Mouse SIRPα Stable Expression Cell Lines Generation

The DNA sequence encoding full length human SIRPα v (NP_542970), cyno SIRPα (NP_001271679), or C57BL/6 mouse SIRPα (NP_031573) was cloned into the pIRES vector (Clontech) respectively. 293F cells (Invitrogen) transfected with human SIRPα v1 expression plasmid were selectively cultured in medium containing 0.5 μg/ml puromycin for 2 weeks. Then single cell clones stably expressing human SIRPα v1 were isolated by limiting dilution and screened by FACS using an anti-human SIRPα antibody (Biolegend, 323802).

In a similar way, CHOK1 cells (Invitrogen) transfected with human SIRPα v1, cyno SIRPα or C57BU/6 mouse SIRPα expression plasmid were selectively cultured in medium containing 6 μg/ml puromycin for 2 weeks. Then single cell clones stably expressing human SIRPα v, cyno SIRPα or C57BL/6 mouse SIRPα were isolated by limiting dilution and screened by FACS using anti-human SIRPα (Biolegend, 323802) or anti-mouse SIRPα (Sino Biological, 50956-R001) antibody.

1.3. Recombinant Proteins Generation

The DNA sequences encoding extracellular domains of human CD47 (NP_001768.1, M1-E141), human SIRPα v1 (NP_542970, M1-R370), human SIRPα v2 (CAA71403.1, M1-R369), human SIRPβ (O00241, M1-L371), or human SIRPγ (Q9P1W8, M1-P360) were cloned into the pCPC vector (Chempartner) expressing human IgG Fc region (hFc). Recombinant ECD protein expressing plasmid transfected Expi293 cells (Invitrogen) were cultured at 37° C. for 1 week. Then the culture medium was collected and centrifuged to remove cell pellets. The harvested supernatant was purified using Protein A affinity chromatography column (Mabselect Sure, GE Healthcare).

Recombinant proteins of 6×His tagged human SIRPα 1 ECD and human SIRPα v8 ECD were purchased from Biointron. Recombinant proteins of 6×His tagged human CD47 ECD, human SIRPα v2 ECD and C57BL/6 mouse SIRPα ECD were purchased from Novoprotein.

### Example 2. Antibody Generation

2.1. Preparation of Immunogen for Protein Immunization

Fc tagged human SIRPα v1 ECD recombinant protein was used as immunogen for protein immunization (refer to Example 1.3).

2.2. Preparation of Immunogen for Cell Immunization

293F cells stably expressing human SIRPα v1 were used as immunogen for cell immunization (refer to Example 1.2).

2.3. Preparation of Immunogen for Genetic Immunization

The DNA sequence encoding full length human SIRPα v1 protein (NP_542970) was cloned into the pCP vector (Chempartner). Then prepared plasmids were coating onto colloidal gold bullets (Bio-Rad) as immunogen for genetic immunization.

2.4. Immunization

Balb/c and SJL/J mice (SLAC) were immunized by three different strategies of protein immunization using human SIRPα v1 ECD recombinant protein, cell immunization using 293F cells stably expressing human SIRPα v1 and genetic immunization using gold bullets coated with human SIRPα v1 expression plasmid. ELISA assay with human SIRPα v1 ECD recombinant protein and FACS assay with 293F cells stably expressing human SIRPα v1 were used to detect serum titer of immunized mice. Mice with high serum titer were selected for hybridoma fusion.

2.5. Hybridoma Generation

5 days after final boost, mice were sacrificed and the spleen cells were collected. 1% (v/v) NH₄OH was added to lyse erythrocytes. Then the washed spleen cells were fused with SP2/0 mouse myeloma cells (ATCC) by high-efficiency electro-fusion or PEG method. After cell fusion, the fused cells were seeded into 96-well plates at the density of 2×10⁴ cells/well with 200 μl DMEM medium containing 20% FBS and 1% HAT.

2.6. Hybridoma Screening

10-12 days after fusion, fusion plates were primarily screened by ELISA assay with human SIRPα v1 ECD recombinant protein or Acumen assay (TTP Labtech) with 293F cells stably expressing human SIRPα v1. The hybridoma cells from positive wells were amplified into 24-well plates for 2^{nd} screening. In 2^{nd} screening, binding activity was assessed by ELISA assay with human SIRPα v1 ECD recombinant protein and FACS assay with 293F cells stably expressing human SIRPα v1. Clones with top binding activity were selected for subclones. In addition, the specificity against human SIRPα v2/β/γ, species cross reactivity, CD47 and SIRPα interaction blocking activity, CD47 and SIRPβ interaction blocking activity were also detected in 2^{nd} screening for hybridoma characterization (refer to Example 3 for methods of the characterization assays).

2.7. Hybridoma Subclone

Hybridoma cells of each selected clone were seeded into 96-well plates at the density of 1 cell/well by limiting dilution. The plates were screened by the same way as hybridoma primary screening (refer to Example 2.6). The positive single clones were picked and characterized by the same way as hybridoma 2^{nd} screening (refer to Example 2.6). Then the monoclonal hybridoma cell lines with top binding activity were obtained for further hybridoma antibody production, characterization and sequencing.

A total of 9 antibody clones were identified as functional hits, and the hybridoma antibodies purified from these clones were assigned as 001, 002, 022, 032, 035, 050, 055, 060, and 074 respectively.

### Example 3. Antibody Characterization

3.1. Hybridoma Antibody Production and Purification

After about 14 days of culturing, the hybridoma cell culture medium was collected and centrifuged to remove cells. After filtered through 0.22 μm PES membrane and adjusting pH to 7.2, the harvested supernatants were loaded to Protein A affinity chromatography column (GE). Antibodies were eluted by 0.1 M citrate sodium buffer (pH3.0) followed by immediately neutralization using Tris buffer (pH8.0). After dialysis with PBS buffer, the antibody concentration was determined by Nano Drop (Thermo Fisher). The purity of proteins was evaluated by SDS-PAGE and HPLC-SEC (Agilent). The endotoxin level was detected with Endochrome-K kit (Charles River).

3.2. Monocyte Derived Macrophage Phagocytosis Assay

The function efficacy of the purified hybridoma antibodies was assessed by a flow cytometry based phagocytosis assay. Briefly, human monocyte derived macrophages were co-cultured with CellTrace Violet (Life Technologies) labeled CD47 expressing cancer cells of Jurkat and Raji in the presence of 50 nM/2 nM anti-SIRPα antibodies. Phagocytosis was assayed by determining the percentage of macrophages positive for cell trace violet dye.

As summarized in Table 7, anti-SIRPα hybridoma antibodies 001, 002, 032, 035, 055, 074, 022, 050, and 060 stimulated potent macrophage phagocytosis of Jurkat cells and Rai cells at the concentration of 2 nM, while other known anti-SIRPα antibodies of 29-AM4-5, KWAR23, and HEFLB showed no or weaker effect. These 9 antibodies were considered as functional antibodies.

TABLE 7
anti-SIRPα hybridoma antibodies characterization summary

| | FACS | | | | | | |
|---|---|---|---|---|---|---|---|
| | CHOK1-human | CHOK1-cyno | CHOK1-C578L/6 | | | | |
| | SIRPα v1 | SIRPα | mouse SIRPα | ELISA (EC50, nM) | | | |
| Antibody | (EC50, nM) | (MFI at 10 nM) | (MFI at 100 nM) | α V1 | α V2 | β | γ |
| 001 | 1.0 | 8030 | − | 0.07 | 0.07 | 0.12 | − |
| 002 | 1.0 | 7540 | − | 0.11 | 0.10 | 0.16 | − |
| 032 | 0.4 | 6864 | − | 0.03 | 0.04 | 0.02 | − |
| 035 | 0.7 | 8306 | 2370 | 0.03 | 0.04 | 0.02 | 2.22 |
| 29-AM4-5 | 13.6 | 37904 | − | 0.15 | 0.20 | 0.21 | 0.22 |
| KWAR23 | 2.8 | 12269 | − | 0.17 | 0.22 | 0.18 | 0.15 |
| HEFLB | 11.2 | − | − | 0.17 | − | 0.17 | 13.4 |
| 055 | 1.2 | 4672 | − | 0.04 | 0.04 | − | − |
| 074 | 1.0 | 14256 | − | 0.02 | 0.02 | 0.02 | − |
| 022 | 2.1 | 6427 | − | 0.04 | 0.04 | 0.03 | 0.76 |
| 050 | 1.3 | 13555 | − | 0.08 | 0.08 | 0.11 | 0.38 |
| 060 | 0.9 | − | − | 0.03 | − | 0.04 | − |

| | Blocking (IC50, nM) | | | Phagocytosis | | |
|---|---|---|---|---|---|---|
| | hSIRPα/ | hSIRPγ/ | | (MDM/Target cell) | | Epitope |
| Antibody | CD47 | CD47 | Hemagglutinatron | Jurkat | Raji | Group |
| 001 | 1.4 | − | − | + | + | I-a |
| 002 | 1.3 | − | − | + | + | I-a |
| 032 | 0.2 | − | − | + | + | I-a |
| 035 | 4.9 | − | − | + | + | I-b |
| 29-AM4-5 | 1.4 | 3.4 | − | − | − | I-a |
| KWAR23 | 0.6 | 1.0 | − | weak | − | I-a |
| HEFLB | 0.5 | 43.2 | − | − | − | I-c |
| 055 | − | − | − | + | + | II |
| 074 | − | − | − | + | + | III |
| 022 | − | − | − | + | + | IV |
| 050 | − | − | − | + | + | IV |
| 060 | >1000 | − | − | + | + | V |

Minus symbol stands for no specific signal or no activity. MFI stands for mean fluorescence intensity in indicated FACS assay. EC50 or IC50 is the concentration of the indicated antibody to reach 50% of the signal in indicated ELISA or blocking assays. Plus symbol stands for the tested antibody alone can stimulate macrophage phagocytosis of tumor cells at the concentration of 2 nM in indicated phagocytosis assays.
(The values may be detected from different batches of experiments.)

3.3. Binding Specificity Detection

Binding specificity of the purified hybridoma antibodies against SIRP family members was detected by ELISA assay using recombinant proteins of Fc tagged human SIRPα v1 ECD, human SIRPα v2 ECD, human SIRPβ ECD and human SIRPγ ECD. Briefly the antibodies were incubated with ELISA microplate coated antigens at 37° C. for 1 hour. After washing, horseradish peroxidase (HRP) labeled anti-mouse or anti-human IgG 2^{nd} Ab (Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read and EC₅₀ was calculated. The binding specificity property of 9 functional antibodies is summarized in Table 7. Other than 060 and HEFLB, all of the antibodies as tested can bind with both human SIRPα v1 and SIRPα v2. 060 and HEFLB can bind with human SIRPα v1 but not v2. Other than 055, all of the antibodies as tested can bind with human SIRPβ. In comparison with other known anti-SIRPα antibodies, only 022, 035 and 050 can bind with human SIRPγ weakly.

3.4. Species Cross Reactivity Testing

Species cross reactivity of the purified hybridoma antibodies against human, cyno and mouse SIRPα was determined by FACS assay using CHOK1-human SIRPα v1-1B4 cells, CHOK1-cyno SIRPα-2A2 cells, and CHOK1-C57BL/6 mouse SIRPα-2.22 cells, which stably expressing SIRPα protein. Briefly the antibodies were incubated with 2×10⁵ target cells at 4° C. for 1 hour. After washing, fluorescence labeled anti-mouse or anti-human IgG 2^{nd} antibody (Life Technologies) was added and incubated at 4° C. for 1 hour. Geometric median fluorescence intensity was detected and EC₅₀ was calculated. The species cross reactivity property of 9 functional antibodies is summarized in Table 7. In particular, it is noted, in contrast to the other antibodies tested in the same experiment, 060 cannot bind with cyno SIRPα and 035 has cross reactivity against C57BL/6 mouse SIRPα.

3.5. CD47/SIRPα, CD47/SIRPγ Interaction Blocking Activity Detection

Competitive ELISA assay was used to determine whether the purified hybridoma antibodies can block CD47 and SIRPα interaction or CD47 and SIRPγ interaction. Briefly, for CD47 and SIRPα interaction blocking activity detection, antibody and biotin labeled soluble human SIRPα v1 ECD recombinant protein were co-incubated with ELISA microplate coated human CD47 ECD recombinant protein.

For CD47 and SIRPγ interaction blocking activity detection, antibody and biotin labeled soluble human CD47 ECD recombinant protein were co-incubated with ELISA microplate coated human SIRPγ ECD recombinant protein. After washing, horseradish peroxidase labeled streptavidin (HRP-SA, Sigma) was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Blocking ratio and IC₅₀ was calculated. The CD47 and SIRPα interaction, CD47 and SIRPγ interaction blocking activity of 9 functional antibodies is summarized in Table 7. In comparison with other known anti-SIRPα antibodies, 022, 050, 055, and 074 cannot block CD47 and SIRPα interaction. In particular, all of the antibodies of the invention cannot block CD47 and SIRPγ interaction.

3.6. Hemagglutination Activity

Anti-CD47 antibodies may promote red blood cell (RBC) hemagglutination, which leads to potential safety risk. The hemagglutination activity of the purified hybridoma antibodies were tested. Briefly, human RBCs were diluted to 10% in PBS and incubated at 37° C. for 1 hour at the presence of 100 nM antibodies. Evidence of hemagglutination is demonstrated by the presence of non-settled RBCs, appearing as a haze compared to punctuate red dot of non-hemagglutinated RBCs. Hemagglutination index was determined by quantitating the area of the RBC pellet in the presence of antibody, normalized to that in the absence of antibody. As summarized in Table 7, all 9 functional antibodies didn't exhibit hemagglutination activity.

3.7. Epitope Binning

Competitive ELISA assay was used for epitope binning of 9 functional antibodies. Briefly excessive competitor antibody and biotin labeled soluble human SIRPα v1 ECD recombinant protein were co-incubated with ELISA microplate coated antibody. After washing, HRP-SA was added and incubated at 37° C. for 1 hour. Then, 100l/well of TMB solution (Biotechnology) was added. After incubation for 15 minutes at room temperature, the reaction was stopped by the addition of 5004 of 1N HCl. OD 450 nm was read. Competition ratio was calculated. The antibodies that can compete each other for binding to SIRPα have the similar binding epitope.

A total of 9 anti-SIRPα antibodies, as shown in Table 8, belong to 5 different epitope groups 001, 002, 032 and 035 belong to the same big group with reference antibodies 29-AM4-5, KWAR23 and HEFLB, which are all CD47 and SIRPα interaction blockers. The other blocker 060 and the non-blockers of 055, 074, 022 and 050 belong to the other four different unique epitope groups.

Specifically, anti-SIRPα antibodies 001, 002, 032 and reference antibodies 29-AM4-5, KWAR23 compete each other for binding to human SIRPα, indicating that they may bind to an identical or closely related epitope which is grouped into I-a as shown in Table 7. Anti-SIRPα antibody 035 also compete with 001, 002 and 032 each other for binding to human SIRPα. However, 035 cannot be fully competed by reference antibodies 29-AM4-5 and KWAR23, indicating that 035 may have a slightly different epitope, which is grouped into I-b as shown in Table 7. Competition between reference antibody HEFLB and anti-SIRPα antibodies 001, 002, 032, 035 is not bidirectional. Thus, the binding epitope of HEFLB is grouped into I-c as show in Table 7. I-a, I-b and I-c are considered to belong to a closely related big group of I. Similarly, antibodies 022 and 050 compete each other for binding to human SIRPα, indicating that they may bind to an identical or closely related epitope which is grouped into IV as shown in Table 7. Antibodies 055, 074, and 060 did not show competitive binding to human SIRPα with any other antibodies in the test, indicating that they may each bind to a different epitope, which is grouped into II, III, and V, respectively, as shown in Table 7.

3.8. Hybridoma Sequencing

RNAs isolated from monoclonal hybridoma cells were reversely transcribed into cDNA using SMARTer RACE 5′/3′ kit (Clontech). Then the cDNA was used as templates to amplify heavy chain and light chain variable region with the primers of Mouse Ig-Primer Set (Novagen). PCR products were analyzed by electrophoresis on agarose gel. DNA fragments with correct size were collected and purified with NucleoSpin Gel and PCR Clear-up kit (MACHEREY NAGEL) followed by ligation with pMD18-T vector (Takara). The ligation products were transformed into DH5α competent cells. Clones were selected and insert fragments were analyzed by DNA sequencing.

### Example 4. Chimeric Antibody Generation and Characterization

4.1. Chimeric Antibody Generation and Production

To validate the result of hybridoma sequencing, mouse antibodies were converted into human IgG4 chimeric antibodies with S228P mutation. Briefly the DNA sequence encoding heavy chain variable region was cloned into the pcDNA3.4-hIgG4P vector (Biointron) carrying human IgG4 heavy chain constant region with S228P mutation. The DNA sequence encoding light chain variable region was cloned into the pcDNA3.4-hIgGk vector (Biointron) carrying human kappa light chain constant region. The resulting chimeric antibodies are referred to herein as 001c, 002c, 022c, 032c, 035c, 050c, 055c, 060c, and 074c, where the suffix “c” indicates chimeric.

TABLE 8
anti-SIRPα hybridoma antibodies epitope binning summary

| Coating mAbs | Competitor mAbs (20 ug), % Competition | | | | | | | | | | | |
|---|---|---|---|---|---|---|---|---|---|---|---|---|
| (0.1 ug) | 001 | 032 | 002 | KWAR23 | HEFLB | 29-AM4-5 | 035 | 055 | 074 | 022 | 050 | 060 |
| 001 | 96 | 96 | 95 | 83 | 36 | 92 | 96 | 4 | 5 | −19 | 5 | 19 |
| 032 | 94 | 94 | 93 | 69 | 14 | 83 | 91 | 3 | 6 | 1 | 4 | 7 |
| 002 | 94 | 92 | 90 | 71 | 7 | 87 | 93 | −16 | −10 | −5 | −13 | −10 |
| KWAR23 | 85 | 82 | 49 | 95 | 27 | 54 | 4 | 1 | 1 | 6 | 5 | 5 |
| HEFLB | 89 | 91 | 81 | 94 | 88 | 70 | 51 | 1 | 2 | 4 | 4 | 7 |
| 29-AM4-S | 97 | 97 | 94 | 93 | 25 | 91 | 98 | 0 | 4 | 8 | 7 | 10 |
| 035 | 92 | 89 | 86 | −6 | 2 | 50 | 93 | −1 | 6 | −1 | −3 | −1 |
| 055 | −3 | −1 | −8 | 45 | −10 | 14 | 8 | 91 | 3 | 2 | 7 | 8 |
| 074 | 33 | 34 | 14 | −14 | −14 | −19 | 10 | −17 | 92 | −5 | 7 | −1 |
| 022 | 2 | 4 | 6 | 1 | 1 | 2 | 0 | 1 | −1 | 81 | 79 | 26 |
| 050 | 2 | 8 | 3 | 0 | −14 | −6 | −4 | −16 | 2 | 79 | 83 | 17 |
| 060 | 0 | 4 | 1 | −4 | −3 | −5 | 3 | 7 | −1 | 10 | 14 | 93 |

Expi293 cells (Life Technologies) co-transfected with antibody heavy and light chain expression plasmids were expanded at 37° C. for 1 week. Then the culture medium was collected and centrifuged to remove cells. The harvested supernatants were loaded to Protein A affinity chromatography column (Nanomicrotech). Antibodies were eluted by 0.1 M citrate sodium buffer (pH3.4) followed by immediately neutralization using Tris buffer (pH8.0). After dialysis with PBS buffer, the antibody concentration was determined by Nano Drop (ThermoFisher). The purity of proteins was evaluated by SDS-PAGE and HPLC-SEC (Agilent). The endotoxin level was detected with Endochrome-K kit (Charles River).

4.2. Chimeric Antibody Characterization

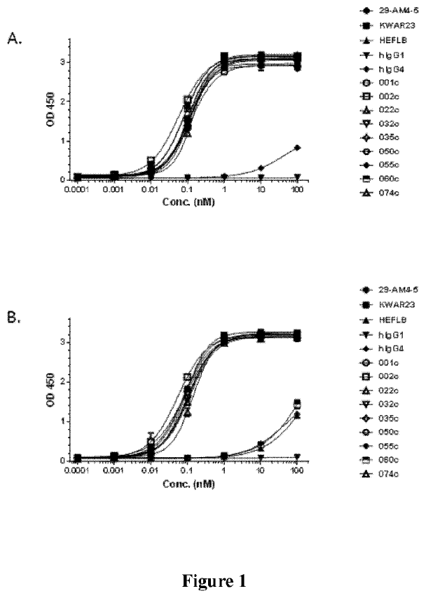

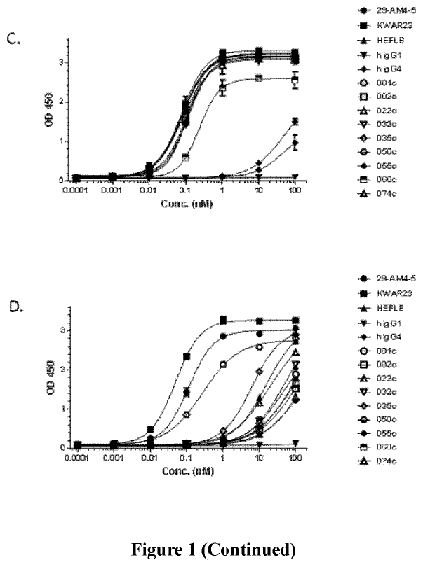

The purified chimeric antibodies were applied for binding specificity analysis and species cross reactivity analysis (refer to methods described in Example 3.3 and 3.4). FIGS. 1A to 1D show binding specificity of anti-SIRPα chimeric antibodies against recombinant proteins of human SIRPα v1 ECD (A), human SIRPα v2 ECD (B), human SIRPβ ECD (C) and human SIRPγ ECD (D) as measured by ELISA analysis.

All the 9 chimeric antibodies as tested showed a subnanomolar EC₅₀ for binding to human SIRPα v1 ECD (FIG. 1 A, Table 9), as measured by ELISA. The reference antibodies 29-AM4-5, KWAR23, and HEFLB also showed similar binding affinity.

Other than 060c and HEFLB, all the other chimeric antibodies and reference antibodies showed a subnanomolar EC₅₀ for binding to human SIRPα v2 ECD (FIG. 1B, Table 10), as measured by ELISA.

Other than 055c, all the other chimeric antibodies and reference antibodies showed a subnanomolar EC₅₀ for binding to human SIRPβ ECD (FIG. 1C, Table 11), as measured by ELISA.

TABLE 9

| Ab | EC₅₀ (nM) |
|---|---|
| 29-AM4-5 | 0.11 |
| KWAR23 | 0.10 |
| HEFLB | 0.11 |
| 001c | 0.12 |
| 002c | 0.06 |
| 022c | 0.11 |
| 032c | 0.12 |
| 035c | 0.08 |
| 050c | 0.11 |
| 055c | 0.11 |
| 060c | 0.08 |
| 074c | 0.14 |

TABLE 10

| Ab | EC₅₀ (nM) |
|---|---|
| 29-AM4-5 | 0.08 |
| KWAR23 | 0.09 |
| HEFLB | N/A |
| 001c | 0.09 |
| 002c | 0.06 |
| 022c | 0.11 |
| 032c | 0.11 |
| 035c | 0.08 |
| 050c | 0.10 |
| 055c | 0.11 |
| 060c | N/A |
| 074c | 0.14 |

TABLE 11

| Ab | EC₅₀ (nM) |
|---|---|
| 29-AM4-5 | 0.08 |
| KWAR23 | 0.08 |
| HEFLB | 0.08 |
| 001c | 0.12 |
| 002c | 0.11 |
| 022c | 0.08 |
| 032c | 0.12 |
| 035c | 0.08 |
| 050c | 0.08 |
| 055c | N/A |
| 060c | 0.24 |
| 074c | 0.11 |

Chimeric antibodies 001c, 002c, 032c, 055c, 060c, 074c did not show specific binding to SIRPγ ECD (FIG. 1D, Table 12), as measured by ELISA. Chimeric antibodies 022c, 035c, and 050c, similar to reference antibodies 29-AM4-5, KWAR23, and HEFLB, all showed specific binding to human SIRPγ ECD (FIG. 1D, Table 12), as measured by ELISA.

TABLE 12

| Ab | EC₅₀ (nM) |
|---|---|
| 29-AM4-5 | 0.11 |
| KWAR23 | 0.05 |
| HEFLB | 15.52 |
| 001c | N/A |
| 002c | N/A |
| 022c | 18.73 |
| 032c | N/A |
| 035c | 6.11 |
| 050c | 0.27 |
| 055c | N/A |
| 060c | N/A |
| 074c | N/A |

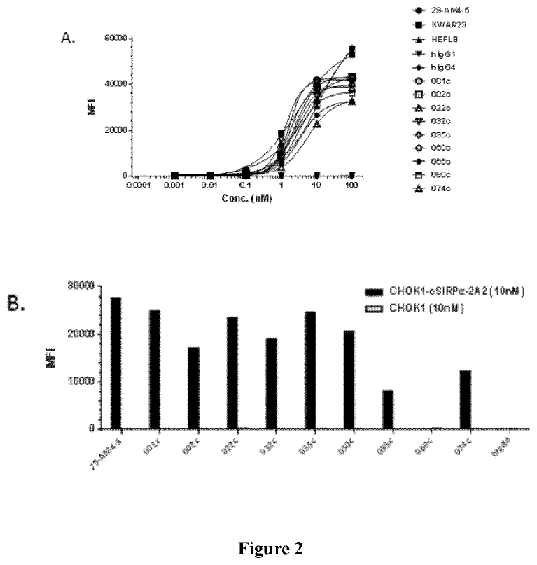

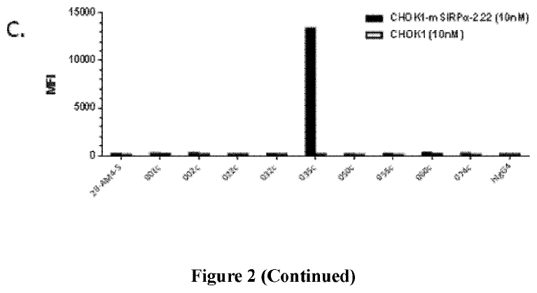

FIGS. 2A to 2C show species cross reactivity of anti-SIRPα chimeric antibodies. FIG. 2A shows the FACS binding curves of the antibodies against CHOK1-human SIRPα v1-1B4 cells. FIGS. 2B and 2C show the FACS binding of 10 nM antibodies against CHOK1-cyno SIRPα-2A2 cells and CHOK1-C57BL/6 mouse SIRPα-2.22 cells.

All the 9 chimeric antibodies as tested showed a subnanomolar EC₅₀ for binding to CHOK1-human SIRPα v1-1B4 cells (FIG. 2A, Table 13), as measured by FACS. The reference antibodies 29-AM4-5, KWAR23, and HEFLB also showed similar binding affinity.

TABLE 13

| Ab | EC₅₀ (nM) |
|---|---|
| 29-AM4-5 | 13.6 |
| KWAR23 | 2.8 |
| HEFLB | 11.2 |
| 001c | 1.7 |
| 002c | 2.4 |
| 022c | 1.7 |
| 032c | 3.0 |
| 035c | 1.3 |
| 050c | 2.4 |
| 055c | 3.4 |
| 060c | 2.8 |
| 074c | 5.5 |

As shown in FIG. 2B, the result indicates that, all antibodies except 060c (i.e. 001c, 002c, 022c, 032c, 035c, 050c, 055c, and 074c) have good cross reactivity against cyno SIRPα. As shown in FIG. 2C only 035c has cross reactivity against mouse SIRPα of C57BL/6 strain.

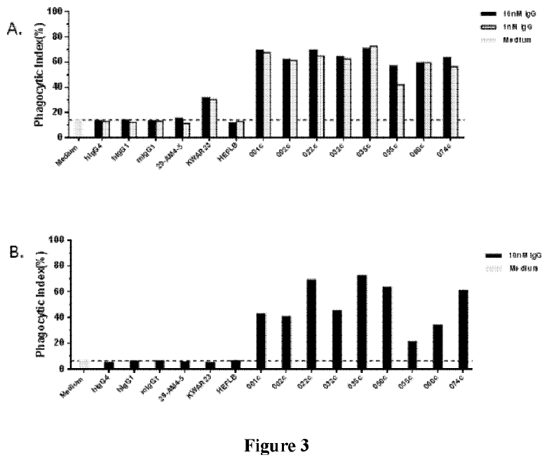

The purified chimeric antibodies were also tested in phagocytosis assay (refer to methods described in Example 3.2). FIGS. 3A to 3D show phagocytosis of Jurkat cells, Raji cells and DLD-1 cells by human macrophages in the presence of the indicated anti-SIRPα antibodies (human IgG4 chimeric antibodies with S228P mutation).

As shown in FIG. 3A to 3D, when used alone, 9 chimeric antibodies stimulated dose dependent potent macrophage phagocytosis of Jurkat cells (FIG. 3A, 3D), Raji cells (FIG. 3B) and DLD-1 cells (FIG. 3C), while the reference antibodies 29-AM4-5, KWAR23, and HEFLB showed no or weaker effect.

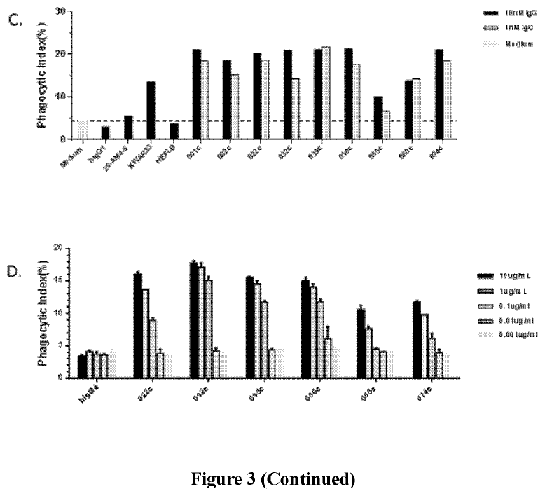

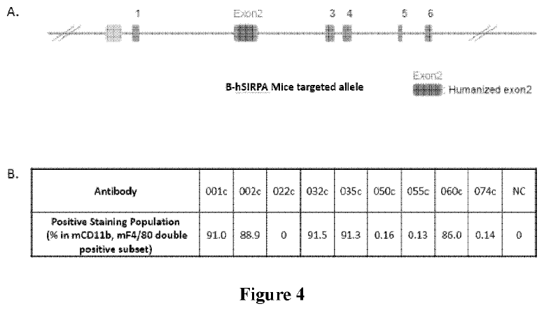

We speculated that the anti-SIRPα chimeric antibodies can block the interaction of CD47 and SIRPα by binding to SIRPα IgV domain, which is the critical region for CD47 interaction. To prove our hypothesis, we tested FACS binding of the anti-SIRPα chimeric antibodies against B-hSIRPA mice (Biocytogen) derived primary monocytes (FIG. 4B).

As shown in FIG. 4A, Sirpa gene exon2 of B-hSIRPA mice, encoding CD47 interacted SIRPα IgV domain, was humanized. The humanized mice express a chimeric SIRPα, comprised of IgV domain of human SIRPα and IgC1/C2, transmembrane and intracellular domains of mouse SIRPα. Briefly spleen cells of B-hSIRPA mice were harvested and incubated with anti-SIRPα chimeric antibodies at 4° C. for 1 hour. After washing, fluorescence labeled anti-human IgG 2^{nd} antibody (Life Technologies) was added and incubated at 4° C. for 1 hour. Mouse CD11b and mouse F4/80 were also stained to demonstrate monocytes. Anti-SIRPα positive staining population in mCD11b and mF4/80 double positive subset was calculated.

As shown in FIG. 4B, CD47 and SIRPα interaction blockers 001c, 002c, 032c, 035c and 060c can bind with B-hSIRPA mice derived primary monocytes, indicating they bind to human SIRPα IgV domain. However, it is not the case for CD47 and SIRPα interaction non-blockers 022c, 050c, 055c and 074c.

All these characterization data are consistent with what we got from hybridoma antibodies, suggesting obtained sequences of variable regions are correct. The characterization data are summarized in Table 14.

TABLE 14
anti-SIRPα chimeric antibodies characterization summary

| | Cross Reactivity (FACS) | | | | | | |
|---|---|---|---|---|---|---|---|
| | CHOK1-human | CHOK1-cyno | CHOK1-C57BL/6 | Specificity (ELISA, EC50, nM) | | | |
| | SIRPα v1 | SIRPα | mouse SIRPα | hSIRPα | hSIRPα | | |
| Antibody | (EC50, nM) | (MFI at 10 nM) | (MFI at 10 nM) | V1 | V2 | hSIIPβ | hSIRPγ |
| 001c | 1.7 | 24911 | − | 0.12 | 0.09 | 0.12 | − |
| 002c | 2.4 | 17074 | − | 0.06 | 0.06 | 0.11 | − |
| 032c | 3.0 | 18967 | − | 0.12 | 0.11 | 0.12 | − |
| 035c | 1.3 | 24634 | 13386 | 0.08 | 0.08 | 0.08 | 6.11 |
| 29-AM4-5 | 13.6 | 27642 | − | 0.11 | 0.08 | 0.08 | 0.11 |
| KWAR23 | 2.8 | 12269 | − | 0.10 | 0.09 | 0.08 | 0.05 |
| HEFLS | 11.2 | − | − | 0.11 | − | 0.08 | 15.52 |
| 055c | 3.4 | 8061 | − | 0.11 | 0.11 | − | − |
| 074c | 5.5 | 12220 | − | 0.14 | 0.14 | 0.11 | − |
| 022c | 1.7 | 23451 | − | 0.11 | 0.11 | 0.08 | 18.73 |
| 050c | 2.4 | 20510 | − | 0.11 | 0.10 | 0.08 | 0.27 |
| 060c | 2.8 | − | − | 0.08 | − | 0.24 | − |

| | | | | Affinity | | Binding | |
|---|---|---|---|---|---|---|---|
| | Phagocytosis (MDM/Target cell) | | | (KD, M) | | Domain | Epitope |
| Antibody | Jurkat | Raji | DLD1 | α V1 | α V2 | IgV | Group |
| 001c | +++ | ++ | ++ | 1.14E−08 | 2.06e−08 | Yes | I-a |
| 002c | +++ | ++ | ++ | 1.61E−08 | 2.83e−08 | Yes | I-a |
| 032c | +++ | ++ | ++ | 8.92E−09 | 1.47e−08 | Yes | I-a |
| 035c | +++ | +++ | ++ | 1.11E−09 | 2.62e−09 | Yes | I-b |
| 29-AM4-5 | − | − | − | N/A | N/A | N/A | I-a |
| KWAR23 | weak | − | + | 2.66E−09 | 1.47E−08 | N/A | I-a |
| HEFLS | − | − | − | N/A | N/A | N/A | I-c |
| 055c | + | + | + | 1.66E−08 | 2.85e−08 | No | II |
| 074c | ++ | +++ | ++ | 4.40E−09 | 2.52e−08 | No | III |
| 022c | ++ | +++ | ++ | 5.90E−09 | 2.56e−08 | No | IV |
| 050c | +++ | +++ | ++ | 3.07E−09 | 4.72e−09 | No | IV |
| 060c | +++ | ++ | + | 1.49E−09 | − | Yes | V |

N/A stands for no available data. Minus symbol stands for no specific signal or no activity. MFI stands for mean fluorescence intensity in indicated FACS assay. EC50 is the concentration of the indicated antibody to reach 50% of the signal in indicated FACS or ELISA assays.
Plus symbol stands for the tested antibody alone can stimulate macrophage phagocytosis of tumor cells at the concentration of 10 nM in indicated phagocytosis assays. Number of plus symbol is used to indicate relative activity level (+++ > ++ > +).

4.3. Binding Affinity Determined by Surface Plasmon Resonance (SPR)

Anti-SIRPα chimeric antibodies were characterized for binding affinity against human SIRPα v1, human SIRPα v2 and C57BL/6 mouse SIRPα using Biacore (GE). Briefly the antibodies to be tested were captured to CM5 chip (GE) using Human Antibody Capture Kit (GE). The antigens of 6×His tagged human SIRPα v1, human SIRPα v2 and C57BL/6 mouse SIRPα ECD recombinant proteins were serially diluted for multiple doses and injected at 30 μl/min for 180 s. Buffer flow was maintained for dissociation of 400 s. 3 M MgCl₂ was used for chip regeneration. The association and dissociation curves were fit with 1:1 binding model, and the Ka/Kd/K_{D} values for each antibody were calculated. The affinity data of anti-SIRPα chimeric antibodies are summarized in Table 15 and Table 14.

### Example 5. Antibody Humanization and Affinity Maturation

5.1 Humanization

The sequences of 035 heavy chain and light chain variable regions were searched in human antibody sequence database. VH7-4-1 and VK1-16 were selected as templates for humanization based on homology to the original mouse antibody sequences. The CDRs from the mouse antibody sequences were then grafted onto the templates, together with the residues to maintain the upper and central core structures of the antibodies. The obtained humanized antibodies for 035 are designated as hu035.01, where the prefix “hu” indicates “humanized”, and the number in the suffix denotes the serial number of the humanized antibody.

TABLE 15
antl-SIRPα chimeric antibodies affinity summary

| | Antigen | | | | | | | | |
|---|---|---|---|---|---|---|---|---|---|
| | hSIRPα V1 | | | hSIRPα V2 | | | mSIRPα (C57BL/6) | | |
| Antibody | ka (1/Ms) | kd (1/s) | KD (M) | ka (1/Ms) | kd (1/s) | KD (M) | ka (1/Ms) | kd (1/s) | KD (M) |
| 001c | 7.49E+04 | 8.55E−04 | 1.14E−08 | 3.97E+04 | 8.16E−04 | 2.06E−08 | − | − | − |
| 002c | 9.20E+04 | 1.48E−03 | 1.61E−08 | 5.16E+04 | 1.46E−03 | 2.83E−08 | − | − | − |
| 022c | 1.25E+05 | 7.40E−04 | 5.90E−09 | 5.61E+04 | 1.44E−03 | 2.56E−08 | − | − | − |
| 032c | 9.35E+04 | 8.34E−04 | 8.92E−09 | 5.26E+04 | 7.74E−04 | 1.47E−08 | − | − | − |
| 035c | 1.83E+05 | 2.03E−04 | 1.11E−09 | 5.90E+04 | 1.54E−04 | 2.62E−09 | 4.69E+03 | 2.47E−02 | 5.27E−06 |
| 050c | 2.52E405 | 7.74E−04 | 3.07E−09 | 1.12E+05 | 5.29E−04 | 4.72E−09 | − | − | − |
| 055c | 8.51E+04 | 1.41E−03 | 1.66E−08 | 4.83E+04 | 1.38E−03 | 2.85E−08 | − | − | − |
| 060c | 9.44E+04 | 1.41E−04 | 1.49E−09 | − | − | − | − | − | − |
| 074c | 5.21E+04 | 2.29E−04 | 4.40E−09 | 2.04E+04 | 5.15E−04 | 2.52E−08 | − | − | − |

Minus symbol stands for the antibody has no detectable interaction with the indicated antigen.

5.2. Humanized Antibody Characterization

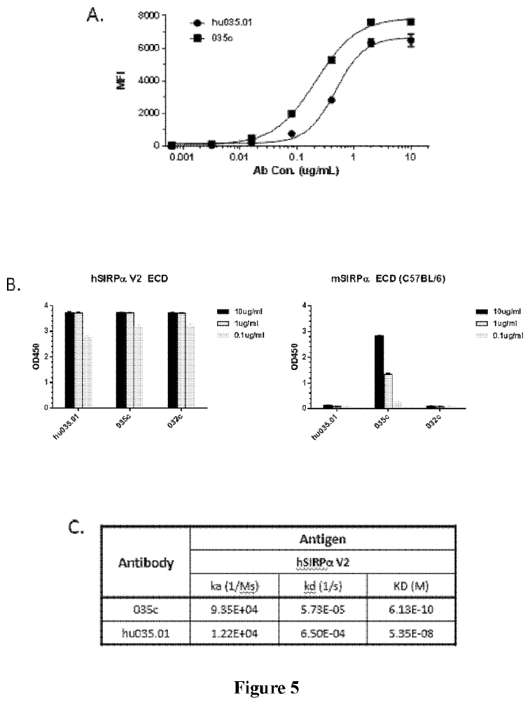

hu035.01, the first version of humanized 035, was characterized by FACS assay using CHOK1-human SIRPα v1-1B4 cells, ELISA assay using Fc tagged human SIRPα v2 ECD recombinant protein and SPR analysis using the antigen of 6×His tagged human SIRPα v2 ECD recombinant protein (refer to methods described in Example 3.4, Example 3.3 and Example 4.3). Compared with the parental antibody of 035c, humanized hu035.01 showed relatively weaker binding against CHOK1-human SIRPα v1-1B4 cells in FACS assay (FIG. 5A) and human SIRPα v2 ECD recombinant protein in ELISA assay (FIG. 5B). SPR analysis with the antigen of human SIRPα v2 ECD recombinant protein confirmed that the binding affinity of hu035.01 (53.4 nM) is lower than 035c (0.61 nM) (FIG. 5C). In particular, hu035.01 cannot be detected to bind with C57BL/6 mouse SIRPα ECD in ELISA assay possibly due to reduced binding activity (FIG. 5B).

5.3. Affinity Maturation

For the reason of reduced binding affinity, hu035.01 was optimized by affinity maturation. Briefly affinity maturation of the first CDR-grafted sequence was done by randomly mutating the heavy and light chains in scFv format and screening for better binders to human SIRPα and/or mouse SIRPα. Top binders were sequenced and cloned into mammalian expression vector, expressed in ExpiCHO cells and purified for further characterization. The obtained humanized antibodies after affinity maturation are designated as hu035.02, hu035.03, throughout to hu035.17, where the prefix “hu” indicates “humanized”, and the number in the suffix denotes the serial number of the humanized antibody.

5.4. Characterization of Humanized Antibodies after Affinity Maturation

The final 7 humanized and matured candidates, which assigned to hu035.02, hu035.03, hu035.09, hu035.10, hu035.13, hu035.14 and hu035.17, were applied for binding specificity analysis and species cross reactivity analysis (refer to methods described in Example 3.3 and 3.4).

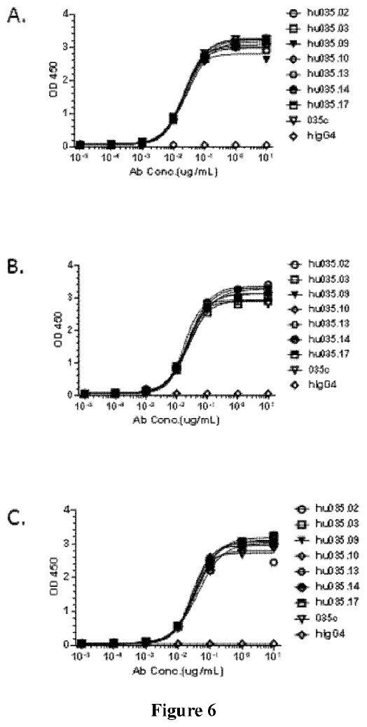

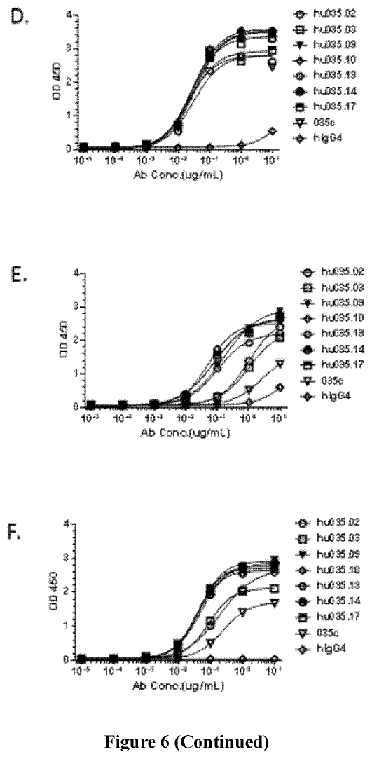

Compared with the parental antibody of 035c, the optimized hu035 candidates were confirmed to maintain comparable binding capability against the recombinant proteins of human SIRPα v1 ECD (FIG. 6A), human SIRPα v2 ECD (FIG. 6B), human SIRPα v8 ECD (FIG. 6C), and human SIRPβ ECD (FIG. 6D), in ELISA assay. In particular, they showed enhanced binding against the recombinant proteins of human SIRPγ ECD (FIG. 6E) and C57BL/6 mouse SIRPα ECD (FIG. 6F) in ELISA assay at different levels. The EC₅₀ values were calculated and summarized in Table 17.

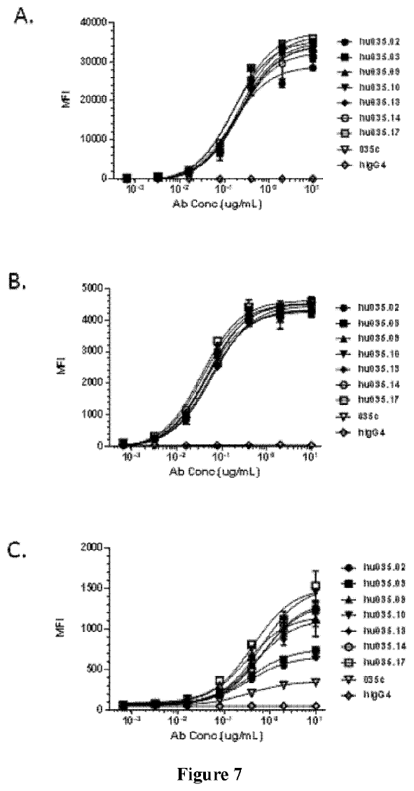

The optimized hu035 candidates were also confirmed to maintain comparable species cross reactivity against human SIRPα (FIG. 7A), cyno SIRPα (FIG. 7B), and C57BL/6 mouse SIRPα (FIG. 7C) by FACS assay. Consistent with the data from ELISA assay, they showed enhanced binding against the CHOK1-C57BL/6 mouse SIRPα-2.22 cells at different levels (FIG. 7C). The EC₅₀ values were calculated and summarized in Table 17.

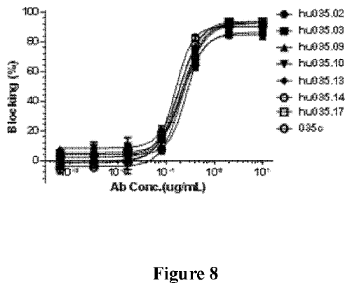

The optimized hu035 candidates were tested for the ability to block CD47 and SIRPα interaction (FIG. 8, refer to methods described in Example 3.5). Compared with the parental antibody of 035c, the optimized hu035 candidates hu035.02, hu035.03, hu035.09, hu035.10, hu035.13, hu035.14 and hu035.17 were confirmed to maintain comparable CD47 and SIRPα interaction blocking activity. The IC₅₀ values were calculated and summarized in Table 17.

SPR analysis further confirmed, compared with the parental antibody of 035c, the optimized hu035 candidates hu035.02, hu035.03, hu035.09, hu035.10, hu035.13, hu035.14 and hu035.17 exhibited comparable binding affinity against human SIRPα alleles, and improved binding affinity against C57BL/6 mouse SIRPα (refer to methods described in Example 4.3). The kinetic data is summarized in Table 16 and Table 17.

TABLE 16
Optimized hu035 candidates affinity kinetics summary

| | Antigen | | | | | | | | |
|---|---|---|---|---|---|---|---|---|---|
| | hSIRPα V1 | | | hSIRPα V2 | | | mSIRPα (C57BL/6) | | |
| Antibody | ka (1/Ms) | kd (1/s) | KD (M) | ka (1/Ms) | kd (1/s) | KD (M) | ka (1/Ms) | kd (1/s) | KD (M) |
| hu035.02 | 1.04E+05 | 1.70E−04 | 1.64E−09 | N/A | N/A | N/A | N/A | N/A | N/A |
| hu035.03 | 5.92E+04 | 1.89E−04 | 3.20E−09 | N/A | N/A | N/A | N/A | N/A | N/A |
| hu035.09 | 8.83E+04 | 1.91E−04 | 2.17E−09 | N/A | N/A | N/A | N/A | N/A | N/A |
| hu035.10 | 6.21E+04 | 2.38E−04 | 3.82E−09 | 1.04E+05 | 1.70E−04 | 1.64E−09 | 4.50E+04 | 1.52E−03 | 3.38E−08 |
| hu035.13 | 1.92E+05 | 1.17E−04 | 6.10E−10 | 5.92E+04 | 1.89E−04 | 3.20E−09 | 5.66E+04 | 2.59E−03 | 4.58E−08 |
| hu035.14 | 8.92E+04 | 1.29E−04 | 1.44E−09 | N/A | N/A | N/A | N/A | N/A | N/A |
| hu035.17 | 1.40E+05 | 1.20E−04 | 8.60E−10 | 8.83E+04 | 1.91E−04 | 2.17E−09 | 5.79E+04 | 1.50E−03 | 2.59E−08 |
| 035c | 1.45E+05 | 2.74E−04 | 1.89E−09 | 6.21E+04 | 2.38E−04 | 3.82E−09 | 4.69E+03 | 2.47E−02 | 5.27E−06 |

N/A stands for no available data.

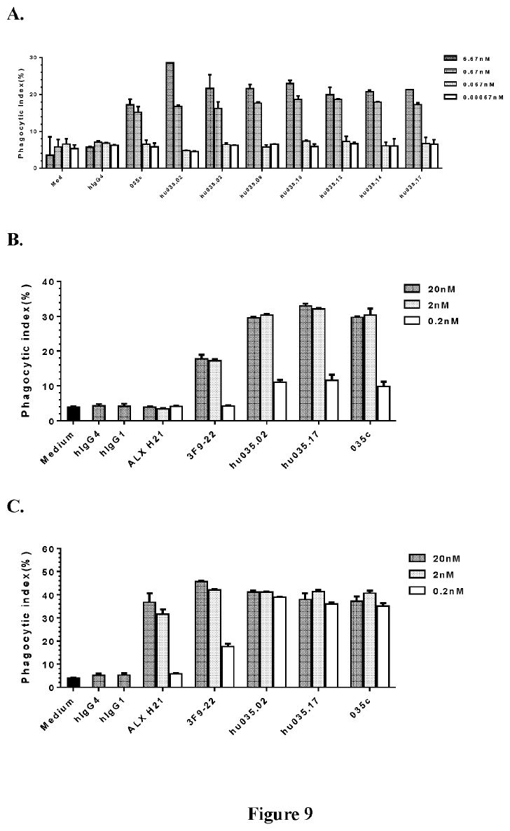

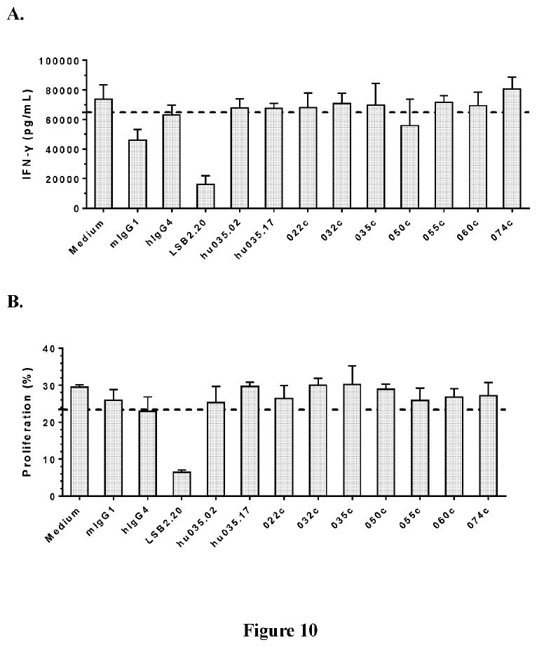

The optimized hu035 candidates were also tested in phagocytosis assay for function evaluation (refer to methods described in Example 3.2). As shown in FIG. 9, compared with the parental antibody of 035c, the optimized hu035 candidates hu035.02, hu035.03, hu035.09, hu035.10, hu035.13, hu035.14 and hu035.17 were confirmed to stimulate stronger or comparable macrophage phagocytosis of Jurkat cells (FIG. 9A), DLD1 cells (FIG. 9B), and Raji cells (FIG. 9C), while two known anti-SIRPα antibodies ALX H21 and 3F9-22 showed no or weaker effect.

It was reported that adhesion of human T cells to antigen-presenting cells through SIRPγ-CD47 interaction co-stimulates T cell proliferation. Since the 7 optimized hu035 candidates showed enhanced binding activity against human SIRP7 compared with the parental antibody of 035c (FIG. 6E), to exclude the possibility of interrupting T cell proliferation, these optimized hu035 candidates as well as some of the chimeric antibodies were tested in T cell activation assays. Briefly, CellTrace Violet (Life Technologies) labeled human primary T cells were stimulated with either ImmunoCul™ Human CD3/CD28 T Cell Activator (STEMCELL) for 4 days, or allogeneic mature dendritic cells generated in vitro for 5 days. Indicated antibodies were added from the beginning of the test at a saturating concentration (10 ug/ml). CellTrace Violet low staining was used to determine proliferation population. IFNγ secretion was determined with human IFN gamma kit (Cisbio).

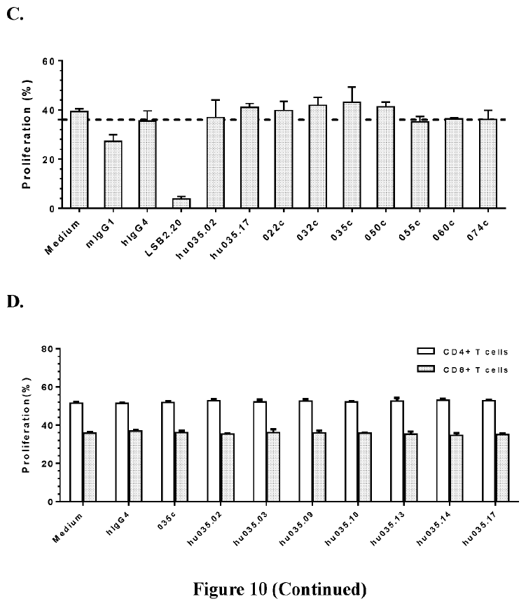

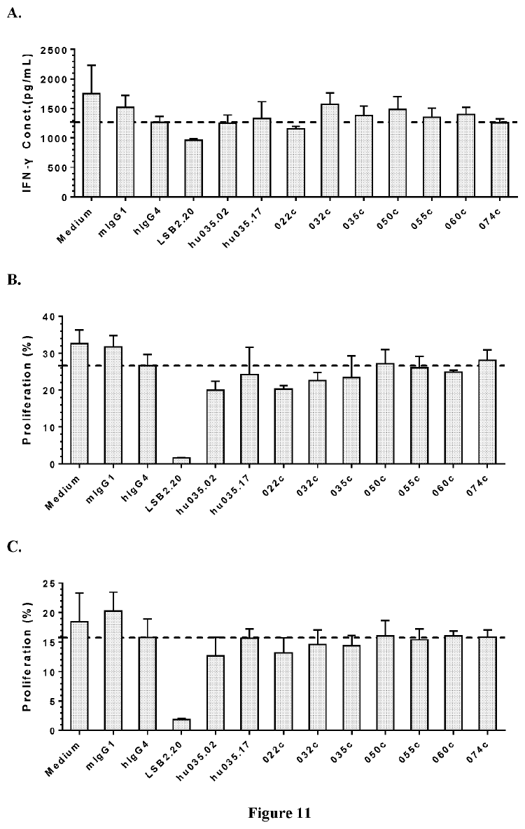

As shown in FIG. 10, the optimized hu035 candidates hu035.02, hu035.03, hu035.09, hu035.10, hu035.13, hu035.14 and hu035.17 as well as chimeric antibodies 035c, 022c, 032c, 050c, 055c, 060c and 074c showed no negative impact on CD4′ T cell proliferation (FIG. 10B, 10D), CD8′ T cell proliferation (FIG. 10C, 10D) when T cells were stimulated with CD3/CD28 T Cell Activator; the optimized hu035 candidates hu035.02 and hu035.17 as well as chimeric antibodies 022c, 032c, 035c, 050c, 055c, 060c and 074c showed no negative impact on IFNγ secretion (FIG. 10A) when T cells were stimulated with CD3/CD28 T Cell Activator. Similarly the optimized hu035 candidates hu035.02, hu035.17 as well as chimeric antibodies 035c, 022c, 032c, 050c, 055c, 060c and 074c showed no negative impact on CD4⁺ T cell proliferation (FIG. 11B), CD8⁺ T cell proliferation (FIG. 11C) and IFNγ secretion (FIG. 11A) when T cells were stimulated with allogeneic dendritic cells. As expected, the anti-SIRPγ antibody LSR2.20 (Biolegend) is an inhibitor of T cell activation.

All of these characterization data is summarized in Table 17, suggesting a successful humanization and affinity maturation.

TABLE 17
Optimized hu035 candidates characterization summary

| | Cross Reactivity (FACS) | | | Specificity | | | |
|---|---|---|---|---|---|---|---|
| | CHOK1-human | CHOK1-cyno | CHOK1-C57BL/6 | (ELISA, EC50, nM) | | | |
| | SIRPα v1 | SIRPα | mouse SIRPα | hSIRPα | hSIRPα | hSIRPα | |
| Antibody | (EC50, nM) | (EC50, nM) | (MFI at 2 ug/ml) | V1 | V2 | V8 | hSIIPβ |
| hu035.02 | 0.97 | 0.29 | 549 | 0.16 | 0.16 | 0.23 | 0.20 |
| hu035.03 | 1.46 | 0.30 | 626 | 0.13 | 0.11 | 0.23 | 0.17 |
| hu035.09 | 1.45 | 0.36 | 950 | 0.13 | 0.19 | 0.21 | 0.23 |
| hu035.10 | 1.44 | 0.33 | 1107 | 0.19 | 0.16 | 0.18 | 0.19 |
| hu035.13 | 1.03 | 0.26 | 876 | 0.14 | 0.17 | 0.16 | 0.18 |
| hu035.14 | 1.31 | 0.36 | 1046 | 0.15 | 0.16 | 0.28 | 0.23 |
| hu035.17 | 1.15 | 0.24 | 1123 | 0.18 | 0.15 | 0.26 | 0.15 |
| 035c | 1.50 | 0.42 | 310 | 0.16 | 0.13 | 0.20 | 0.13 |

| | | Phagocytosis | | | | |
|---|---|---|---|---|---|---|
| | | (MDM/Jurkat, | Affinity | | | |
| | Specificity | phagocytosis | (KD, M) | | | hSIRPα/CD47 |
| | (ELISA, EC50, nM) | Index at | hSIRPα | hSIRPα | Mouse | Blocking |
| Antibody | hSIRPγ | 0.1 ug/ml, %) | V1 | V2 | (C57BL/6) | (IC50, nM) |
| hu035.02 | 6.24 | 16.76 | 1.64E−09 | N/A | N/A | 1.74 |
| hu035.03 | 6.78 | 16.25 | 3.20E−09 | N/A | N/A | 1.43 |
| hu035.09 | 1.05 | 17.67 | 2.17E−09 | N/A | N/A | 1.36 |
| hu035.10 | 0.34 | 18.65 | 3.82E−09 | 2.15E−09 | 3.38E−08 | 1.49 |
| hu035.13 | 0.67 | 18.64 | 6.10E−10 | 1.24E−09 | 4.58E−08 | 1.33 |
| hu035.14 | 0.52 | 18.0 | 1.44E−09 | N/A | N/A | 1.46 |
| hu035.17 | 0.54 | 17.3 | 8.60E−10 | 1.43E−09 | 2.59E−08 | 1.22 |
| 035c | 16.44 | 15.13 | 1.89E−09 | 1.39E−09 | 5.27E−06 | 1.03 |

N/A stands for no available data.
MFI stands for mean fluorescence intensity in indicated FACS assays. EC50 or IC50 is the concentration of the indicated antibody to reach 50% of the signal in indicated ELISA, FACS or blocking assays.

1. An antibody or an antigen-binding fragment thereof capable of specifically binding to human SIRPα, comprising a heavy chain variable region comprising HCDR1, HCDR2 and HCDR3, and/or a light chain variable region comprising LCDR1, LCDR2 and LCDR3, wherein
a) the HCDR1 comprises a sequence selected from the group consisting of RNYWMN (SEQ ID NO: 1), TDYAMH (SEQ ID NO: 2), TX₁YAMN (SEQ ID NO: 3), THYSMH (SEQ ID NO: 4), SDYFMT (SEQ ID NO: 5), TNYDIS (SEQ ID NO: 6), SSYWIH (SEQ ID NO: 7); and
b) the HCDR2 comprises a sequence selected from the group consisting of EIX₂LKSNTYATHYAESVKG (SEQ ID NO: 8), WKNTETGESTYAEDFKG (SEQ ID NO: 9), X₃INTYTGEPTYAX₄X₅FKG (SEQ ID NO: 10), WINTETAEPTYVDDFKG (SEQ ID NO: 11), NVNYDGRSTYYLDSLKS (SEQ ID NO: 12), VIWTGGDTNFNSAFMS (SEQ ID NO: 13), or LIHPNSGNTDCSETFKN (SEQ ID NO: 14); and
c) the HCDR3 comprises a sequence selected from the group consisting of FTKVVADWHLDV (SEQ ID NO: 15), GGYGSNYVMDY (SEQ ID NO: 16), TRGYYDFDGGAFDY (SEQ ID NO: 17), GGLRQGDY (SEQ ID NO: 18), EGSQTPLYAVDY (SEQ ID NO: 19), VQYFGGSYGPMDY (SEQ ID NO: 20), DGASYDWFVH (SEQ ID NO: 21); and
d) the LCDR1 comprises a sequence selected from the group consisting of RSSQNIVHSNGNTYLE (SEQ ID NO: 22), KASEDIYNRLA (SEQ ID NO: 23), X₆ASQNVGTHLA (SEQ ID NO: 24), SATSSVSASYLY (SEQ ID NO: 25), KASQNVGTAVA (SEQ ID NO: 26), EASDHINDWLA (SEQ ID NO: 27), KSSQSLLYTNGKTYLN (SEQ ID NO: 28); and
e) the LCDR2 comprises a sequence selected from the group consisting of KX₇SNRFS (SEQ ID NO: 29), GATSLET (SEQ ID NO: 30), SAX₈YRYI (SEQ ID NO: 31), STSNLAS (SEQ ID NO: 32), LASNRYT (SEQ ID NO: 33), LVSKLDS (SEQ ID NO: 35); and
f) the LCDR3 comprises a sequence selected from the group consisting of FQGSHVPFT (SEQ ID NO: 36), QQYWNSPRT (SEQ ID NO: 37), QQYNTYPLT (SEQ ID NO: 38), HQWSSYPYT (SEQ ID NO: 39), QQYSIYPFT (SEQ ID NO: 40), QQYWNTPLT (SEQ ID NO: 41), VQGTHFPRT (SEQ ID NO: 42),
wherein X₁ is N or D, X₂ is S or T, X₃ is F or W, X₄ is Q or D, X₅ is D or G, X₆ is K or R, X₇ is V or I, X₈ is S or I.

2. The antibody or antigen-binding fragment thereof of claim 1, wherein
the HCDR1 comprises an amino acid sequence of SEQ ID NO: 1, and/or
the HCDR2 comprises an amino acid sequence of SEQ ID NO: 8, and/or
the HCDR3 comprises an amino acid sequence of SEQ ID NO: 15, and/or
the LCDR1 comprises an amino acid sequence of SEQ ID NO: 22, and/or
the LCDR2 comprises an amino acid sequence of SEQ ID NO: 29, and/or
the LCDR3 comprises an amino acid sequence of SEQ ID NO: 36.

3. (canceled)

4. The antibody or an antigen-binding fragment thereof of claim 1, wherein
the HCDR1 comprises an amino acid sequence of SEQ ID NO: 3, and/or
the HCDR2 comprises an amino acid sequence of SEQ ID NO: 10, and/or
the HCDR3 comprises an amino acid sequence of SEQ ID NO: 17, and/or
the LCDR1 comprises an amino acid sequence of SEQ ID NO: 24, and/or
the LCDR2 comprises an amino acid sequence of SEQ ID NO: 31, and/or
the LCDR3 comprises an amino acid sequence of SEQ ID NO: 38.

5. The antibody or an antigen-binding fragment thereof of claim 4, wherein
a) the HCDR1 comprises an amino acid sequence selected from the group consisting of TNYAMN (SEQ ID NO: 43) and TDYAMN (SEQ ID NO: 45), and/or
b) the HCDR2 comprises an amino acid sequence selected from the group consisting of FINTYTGEPTYADDFKG (SEQ ID NO: 50), WINTYTGEPTYAQGFKG (SEQ ID NO: 51), and FINTYTGEPTYAQGFKG (SEQ ID NO: 52), and/or
c) the HCDR3 comprises an amino acid sequence of SEQ ID NO: 17, and/or
d) the LCDR1 comprises an amino acid sequence selected from the group consisting of KASQNVGTHLA (SEQ ID NO: 53), and RASQNVGTHLA (SEQ ID NO: 54), and/or
e) the LCDR2 comprises an amino acid sequence selected from the group consisting of SASYRYI (SEQ ID NO: 57) and SAIYRYI (SEQ ID NO: 58), and/or
f) the LCDR3 comprises an amino acid sequence of SEQ ID NO: 38.

6. The antibody or an antigen-binding fragment thereof of claim 1, wherein the heavy chain variable region comprises:
a) a HCDR1 comprising the sequence of SEQ ID NO: 1, a HCDR2 comprising the sequence of SEQ ID NO: 48, and a HCDR3 comprising the sequence of SEQ ID NO: 15; or
b) a HCDR1 comprising the sequence of SEQ ID NO: 1, a HCDR2 comprising the sequence of SEQ ID NO: 49, and a HCDR3 comprising the sequence of SEQ ID NO: 15; or
c) a HCDR1 comprising the sequence of SEQ ID NO: 2, a HCDR2 comprising the sequence of SEQ ID NO: 9, and a HCDR3 comprising the sequence of SEQ ID NO: 16; or
d) a HCDR1 comprising the sequence of SEQ ID NO: 43, a HCDR2 comprising the sequence of SEQ ID NO: 50, and a HCDR3 comprising the sequence of SEQ ID NO: 17; or
e) a HCDR1 comprising the sequence of SEQ ID NO: 43, a HCDR2 comprising the sequence of SEQ ID NO: 51, and a HCDR3 comprising the sequence of SEQ ID NO: 17; or
f) a HCDR1 comprising the sequence of SEQ ID NO: 45, a HCDR2 comprising the sequence of SEQ ID NO: 52, and a HCDR3 comprising the sequence of SEQ ID NO: 17; or
g) a HCDR1 comprising the sequence of SEQ ID NO: 43, a HCDR2 comprising the sequence of SEQ ID NO: 52, and a HCDR3 comprising the sequence of SEQ ID NO: 17; or
h) a HCDR1 comprising the sequence of SEQ ID NO: 4, a HCDR2 comprising the sequence of SEQ ID NO: 11, and a HCDR3 comprising the sequence of SEQ ID NO: 18; or
i) a HCDR1 comprising the sequence of SEQ ID NO: 5, a HCDR2 comprising the sequence of SEQ ID NO: 12, and a HCDR3 comprising the sequence of SEQ ID NO: 19; or
j) a HCDR1 comprising the sequence of SEQ ID NO: 6, a HCDR2 comprising the sequence of SEQ ID NO: 13, and a HCDR3 comprising the sequence of SEQ ID NO: 20; or
k) a HCDR1 comprising the sequence of SEQ ID NO: 7, a HCDR2 comprising the sequence of SEQ ID NO: 14, and a HCDR3 comprising the sequence of SEQ ID NO: 21.

7. The antibody or an antigen-binding fragment thereof of claim 1, wherein the light chain variable region comprises:
a) a LCDR1 comprising the sequence of SEQ ID NO: 22, a LCDR2 comprising the sequence of SEQ ID NO: 55, and a LCDR3 comprising the sequence of SEQ ID NO: 36; or
b) a LCDR1 comprising the sequence of SEQ ID NO: 22, a LCDR2 comprising the sequence of SEQ ID NO: 56, and a LCDR3 comprising the sequence of SEQ ID NO: 36; or
c) a LCDR1 comprising the sequence of SEQ ID NO: 23, a LCDR2 comprising the sequence of SEQ ID NO: 30, and a LCDR3 comprising the sequence of SEQ ID NO: 37; or
d) a LCDR1 comprising the sequence of SEQ ID NO: 53, a LCDR2 comprising the sequence of SEQ ID NO: 57, and a LCDR3 comprising the sequence of SEQ ID NO: 38; or
e) a LCDR1 comprising the sequence of SEQ ID NO: 54, a LCDR2 comprising the sequence of SEQ ID NO: 57, and a LCDR3 comprising the sequence of SEQ ID NO: 38; or
f) a LCDR1 comprising the sequence of SEQ ID NO: 54, a LCDR2 comprising the sequence of SEQ ID NO: 58, and a LCDR3 comprising the sequence of SEQ ID NO: 38; or
g) a LCDR1 comprising the sequence of SEQ ID NO: 25, a LCDR2 comprising the sequence of SEQ ID NO: 32, and a LCDR3 comprising the sequence of SEQ ID NO: 39; or
h) a LCDR1 comprising the sequence of SEQ ID NO: 26, a LCDR2 comprising the sequence of SEQ ID NO: 33, and a LCDR3 comprising the sequence of SEQ ID NO: 40; or
i) a LCDR1 comprising the sequence of SEQ ID NO: 27, a LCDR2 comprising the sequence of SEQ ID NO: 30, and a LCDR3 comprising the sequence of SEQ ID NO: 41; or
j) a LCDR1 comprising the sequence of SEQ ID NO: 28, a LCDR2 comprising the sequence of SEQ ID NO: 35, and a LCDR3 comprising the sequence of SEQ ID NO: 42.

8. The antibody or an antigen-binding fragment thereof of claim 1, wherein
a) the HCDR1 comprises the sequence of SEQ ID NO: 1, the HCDR2 comprises the sequence of SEQ ID NO: 48, the HCDR3 comprises the sequence of SEQ ID NO: 15, the LCDR1 comprises the sequence of SEQ ID NO: 22, the LCDR2 comprises the sequence of SEQ ID NO: 55, and the LCDR3 comprises the sequence of SEQ ID NO: 36: or
b) the HCDR1 comprises the sequence of SEQ ID NO: 1, the HCDR2 comprises the sequence of SEQ ID NO: 49, the HCDR3 comprises the sequence of SEQ ID NO: 15, the LCDR1 comprises the sequence of SEQ ID NO: 22, the LCDR2 comprises the sequence of SEQ ID NO: 56, and the LCDR3 comprises the sequence of SEQ ID NO: 36, or
c) the HCDR1 comprises the sequence of SEQ ID NO: 1, the HCDR2 comprises the sequence of SEQ ID NO: 49, the HCDR3 comprises the sequence of SEQ ID NO: 15, the LCDR1 comprises the sequence of SEQ ID NO: 22, the LCDR2 comprises the sequence of SEQ ID NO: 55, and the LCDR3 comprises the sequence of SEQ ID NO: 36; or
d) the HCDR1 comprises the sequence of SEQ ID NO: 2, the HCDR2 comprises the sequence of SEQ ID NO: 9, the HCDR3 comprises the sequence of SEQ ID NO: 16, the LCDR1 comprises the sequence of SEQ ID NO: 23, the LCDR2 comprises the sequence of SEQ ID NO: 30, and the LCDR3 comprises the sequence of SEQ ID NO: 37; or
e) the HCDR1 comprises the sequence of SEQ ID NO: 43, the HCDR2 comprises the sequence of SEQ ID NO: 50, the HCDR3 comprises the sequence of SEQ ID NO: 17, the LCDR1 comprises the sequence of SEQ ID NO: 53, the LCDR2 comprises the sequence of SEQ ID NO: 57, and the LCDR3 comprises the sequence of SEQ ID NO: 38; or
f) the HCDR1 comprises the sequence of SEQ ID NO: 43, the HCDR2 comprises the sequence of SEQ ID NO: 51, the HCDR3 comprises the sequence of SEQ ID NO: 17, the LCDR1 comprises the sequence of SEQ ID NO: 54, the LCDR2 comprises the sequence of SEQ ID NO: 57, and the LCDR3 comprises the sequence of SEQ ID NO: 38; or
g) the HCDR1 comprises the sequence of SEQ ID NO: 45, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 57, and the LCDR3 comprises the sequence of SEQ ID NO: 38; or
h) the HCDR1 comprises the sequence of SEQ ID NO: 45, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 58, and the LCDR3 comprises the sequence of SEQ ID NO: 38; or
i) the HCDR1 comprises the sequence of SEQ ID NO: 43, the HCDR2 comprises the sequence of SEQ ID NO: 52, the HCDR3 comprises the sequence of SEQ ID NO: 17, the LCDR1 comprises the sequence of SEQ ID NO: 54, the LCDR2 comprises the sequence of SEQ ID NO: 58, and the LCDR3 comprises the sequence of SEQ ID NO: 38; or
j) the HCDR1 comprises the sequence of SEQ ID NO: 4, the HCDR2 comprises the sequence of SEQ ID NO: 11, the HCDR3 comprises the sequence of SEQ ID NO: 18, the LCDR1 comprises the sequence of SEQ ID NO: 25, the LCDR2 comprises the sequence of SEQ ID NO: 32, and the LCDR3 comprises the sequence of SEQ ID NO: 39, or
k) the HCDR1 comprises the sequence of SEQ ID NO: 5, the HCDR2 comprises the sequence of SEQ ID NO: 12, the HCDR3 comprises the sequence of SEQ ID NO: 19, the LCDR1 comprises the sequence of SEQ ID NO: 26, the LCDR2 comprises the sequence of SEQ ID NO: 33, and the LCDR3 comprises the sequence of SEQ ID NO: 40; or
l) the HCDR1 comprises the sequence of SEQ ID NO: 6, the HCDR2 comprises the sequence of SEQ ID NO: 13, the HCDR3 comprises the sequence of SEQ ID NO: 20, the LCDR1 comprises the sequence of SEQ ID NO: 27, the LCDR2 comprises the sequence of SEQ ID NO: 30, and the LCDR3 comprises the sequence of SEQ ID NO: 41; or
m) the HCDR1 comprises the sequence of SEQ ID NO: 7, the HCDR2 comprises the sequence of SEQ ID NO: 14, the HCDR3 comprises the sequence of SEQ ID NO: 21, the LCDR1 comprises the sequence of SEQ ID NO: 28, the LCDR2 comprises the sequence of SEQ ID NO: 35, and the LCDR3 comprises the sequence of SEQ ID NO: 42.

9. The antibody or an antigen-binding fragment thereof of claim 1, further comprising one or more of heavy chain HFR1, HFR2, HFR3 and HFR4, and/or one or more of light chain LFR1, LFR2, LFR3 and LFR4, wherein:
a) the HFR1 comprises QX₉QLVQSGSELKKPGASVKVSCX₁₀AX₁₁GYX₁₂X₁₃ (SEQ ID NO: 92) or a homologous sequence of at least 80% sequence identity thereof,
b) the HFR2 comprises WVRQAPGQGLEWMG (SEQ ID NO: 93) or a homologous sequence of at least 80% sequence identity thereof,
c) the HFR3 sequence comprises RFVFSLDTSVSTAYLQIX₁₄SLKAEDTAVYYCAR (SEQ ID NO: 96) or a homologous sequence of at least 80% sequence identity thereof,
d) the HFR4 comprises WGQGTLVTVSS (SEQ ID NO: 97) or a homologous sequence of at least 80% sequence identity thereof,
e) the LFR1 comprises DIQMTQSPSX₁₅LX₁₆ASVGDRVTITC (SEQ ID NO: 100) or a homologous sequence of at least 80% sequence identity thereof,
f) the LFR2 comprises WX₁₇QQKPGKX₁₈PKX₁₉LIX₂₀ (SEQ ID NO: 104) or a homologous sequence of at least 80% sequence identity thereof,
g) the LFR3 comprises GVPSRFSGSGSGTDFTLTISX₂₁LQPEDFATYX₂₂C (SEQ ID NO: 108) or a homologous sequence of at least 80% sequence identity thereof, and
h) the LFR4 comprises FX₂₃QGTKLEIKX₂₄ (SEQ ID NO: 47) or a homologous sequence of at least 80% sequence identity thereof,
wherein X₉ is I or V, X₁₀ is R or K, X₁₁ is G or R or S, X₁₂ is T or S, X₁₃ is L or I or F, X₁₄ is G or S, X₁₅ is S or R, X₁₆ is S or G, X₁₇ is Y or F, X₁₈ is A or S, X₁₉ is S or A, X₂₀ is Y or F, X₂₁ is S or N, X₂₂ is Y or F, X₂₃ is G or D, X₂₄ is R or absent.

10. (canceled)

11. The antibody or an antigen-binding fragment thereof of claim 1, wherein the heavy chain variable region comprises the sequence selected from the group consisting of SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and a homologous sequence thereof having at least 80% sequence identity yet retaining specific binding affinity to human SIRPα, and/or the light chain variable region comprises the sequence selected from the group consisting of SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, and a homologous sequence thereof having at least 80% sequence identity yet retaining specific binding affinity to human SIRPα.

12. (canceled)

13. The antibody or an antigen-binding fragment thereof of claim 1, wherein
a) the heavy chain variable region comprises the sequence of SEQ ID NO: 59 and the light chain variable region comprises the sequence of SEQ ID NO: 73; or
b) the heavy chain variable region comprises the sequence of SEQ ID NO: 60 and the light chain variable region comprises the sequence of SEQ ID NO: 74; or
c) the heavy chain variable region comprises the sequence of SEQ ID NO: 61 and the light chain variable region comprises the sequence of SEQ ID NO: 75; or
d) the heavy chain variable region comprises the sequence of SEQ ID NO: 62 and the light chain variable region comprises the sequence of SEQ ID NO: 76; or
e) the heavy chain variable region comprises the sequence of SEQ ID NO: 63 and the light chain variable region comprises the sequence of SEQ ID NO: 77; or
f) the heavy chain variable region comprises the sequence of SEQ ID NO: 64 and the light chain variable region comprises the sequence of SEQ ID NO: 78; or
g) the heavy chain variable region comprises the sequence of SEQ ID NO: 65 and the light chain variable region comprises the sequence of SEQ ID NO: 79; or
h) the heavy chain variable region comprises the sequence of SEQ ID NO: 65 and the light chain variable region comprises the sequence of SEQ ID NO: 80; or
i) the heavy chain variable region comprises the sequence of SEQ ID NO: 66 and the light chain variable region comprises the sequence of SEQ ID NO: 81; or
j) the heavy chain variable region comprises the sequence of SEQ ID NO: 65 and the light chain variable region comprises the sequence of SEQ ID NO: 82; or
k) the heavy chain variable region comprises the sequence of SEQ ID NO: 67 and the light chain variable region comprises the sequence of SEQ ID NO: 83; or
l) the heavy chain variable region comprises the sequence of SEQ ID NO: 68 and the light chain variable region comprises the sequence of SEQ ID NO: 82; or
m) the heavy chain variable region comprises the sequence of SEQ ID NO: 65 and the light chain variable region comprises the sequence of SEQ ID NO: 84; or
n) the heavy chain variable region comprises the sequence of SEQ ID NO: 69 and the light chain variable region comprises the sequence of SEQ ID NO: 85; or
o) the heavy chain variable region comprises the sequence of SEQ ID NO: 70 and the light chain variable region comprises the sequence of SEQ ID NO: 86; or
p) the heavy chain variable region comprises the sequence of SEQ ID NO: 71 and the light chain variable region comprises the sequence of SEQ ID NO: 87; or
q) the heavy chain variable region comprises the sequence of SEQ ID NO: 72 and the light chain variable region comprises the sequence of SEQ ID NO. 88.

14. The antibody or an antigen-binding fragment thereof of claim 1, further comprising one or more amino acid residue substitutions or modifications yet retains specific binding affinity to human SIRPα.

15. The antibody or an antigen-binding fragment thereof of claim 14, wherein at least one of the substitutions or modifications is in one or more of the CDR sequences, and/or in one or more of the non-CDR sequences of the heavy chain variable region or light chain variable region.

16. The antibody or an antigen-binding fragment thereof of claim 1, further comprising an Fc region, optionally an Fc region of human immunoglobulin (Ig), or optionally an Fc region of human IgG, or optionally an Fc region derived from human IgG4, or optionally an Fc region derived from human IgG4 comprising a S228P mutation and/or a L235E mutation.

17-18. (canceled)

19. The antibody or an antigen-binding fragment thereof of claim 1, which is humanized.

20. The antibody or an antigen-binding fragment thereof of claim 1, which is a monoclonal antibody, a bispecific antibody, a multi-specific antibody, a recombinant antibody, a chimeric antibody, a labeled antibody, a bivalent antibody, an anti-idiotypic antibody or a fusion protein.

21. The antibody or an antigen-binding fragment thereof of claim 1, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

22-23. (canceled)

24. An anti-SIRPα antibody or an antigen-binding fragment thereof, which competes for binding to human SIRPα with the antibody or an antigen-binding fragment thereof of claim 1.

25. The antibody or an antigen-binding fragment thereof of claim 24, which competes for binding to human SIRPα with (i) an antibody comprising a heavy chain variable region comprising the sequence of SEQ ID NO: 70, and a light chain variable region comprising the sequence of SEQ ID NO: 86; (ii) an antibody comprising a heavy chain variable region comprising the sequence of SEQ ID NO: 72, and a light chain variable region comprising the sequence of SEQ ID NO: 88; (iii) an antibody comprising a heavy chain variable region comprising the sequence of SEQ ID NO: 62, and a light chain variable region comprising the sequence of SEQ ID NO: 76; (iv) an antibody comprising a heavy chain variable region comprising the sequence of SEQ ID NO: 69, and a light chain variable region comprising the sequence of SEQ ID NO: 85; or (v) an antibody comprising a heavy chain variable region comprising the sequence of SEQ ID NO: 71, and a light chain variable region comprising the sequence of SEQ ID NO: 87.

26-32. (canceled)

33. The antibody or an antigen-binding fragment thereof of claim 1, which is linked to one or more conjugate moieties.

34. (canceled)

35. A pharmaceutical composition comprising the antibody or an antigen-binding fragment thereof of claim 1, and one or more pharmaceutically acceptable carriers.

36. An isolated polynucleotide encoding the antibody or an antigen-binding fragment thereof of claim 1.

37. A vector comprising the isolated polynucleotide of claim 36.

38. A host cell comprising the vector of claim 37.

39. A kit comprising the antibody or an antigen-binding fragment thereof of claim 1 and/or a pharmaceutical composition comprising the antibody or antigen-binding fragment thereof and one or more pharmaceutically acceptable carriers, and a second therapeutic agent.

40. A method of expressing the antibody or an antigen-binding fragment thereof of claim 1, comprising culturing the host cell comprising a vector comprising an isolated polynucleotide encoding the antibody or antigen-binding fragment thereof under the condition at which the vector is expressed.

41. A method of treating, preventing or alleviating a SIRPα related disease, disorder or condition in a subject, comprising administering to the subject a therapeutically effective amount of the antibody or an antigen-binding fragment thereof of claim 1 and/or a pharmaceutical composition comprising the antibody or antigen-binding fragment thereof and one or more pharmaceutically acceptable carriers.

42. The method of claim 41, wherein the disease, disorder or condition is cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis.

43. The method of claim 42, wherein the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, and adenocarcinoma.

44-46. (canceled)

47. The method of claim 41, further comprising administering a therapeutically effective amount of a second therapeutic agent.

48. (canceled)

49. A method of modulating SIRPα activity in a SIRPα-positive cell, comprising exposing the SIRPα-positive cell to the antibody or antigen-binding fragment thereof of claim 1 and/or a pharmaceutical composition comprising the antibody or antigen-binding fragment thereof and one or more pharmaceutically acceptable carriers.

50. (canceled)

51. A method of detecting the presence or amount of SIRPα in a sample, comprising contacting the sample with the antibody or an antigen-binding fragment thereof of claim 1 and/or a pharmaceutical composition comprising the antibody or antigen-binding fragment thereof and one or more pharmaceutically acceptable carriers, and determining the presence or the amount of SIRPα in the sample.

52. A method of diagnosing a SIRPα related disease, disorder or condition in a subject, comprising: a) contacting a sample obtained from the subject with the antibody or an antigen-binding fragment thereof of claim 1 and/or a pharmaceutical composition comprising the antibody or antigen-binding fragment thereof and one or more pharmaceutically acceptable carriers; b) determining the presence or amount of SIRPα in the sample; and c) correlating the presence or the amount of SIRPα to existence or status of the SIRPα related disease, disorder or condition in the subject.

53-56. (canceled)

57. A kit comprising the antibody or an antigen-binding fragment thereof of claim 1 and/or a pharmaceutical composition comprising the antibody or antigen-binding fragment thereof and one or more pharmaceutically acceptable carriers, useful in detecting SIRPα.

58. (canceled)

59. A method of inducing phagocytosis in a subject, comprising administering to the subject the antibody or an antigen-binding fragment thereof of claim 1 and/or a pharmaceutical composition comprising the antibody or antigen-binding fragment thereof and one or more pharmaceutically acceptable carriers in a dose effective to induce phagocytosis.

60-61. (canceled)

62. A method of inducing phagocytosis in vitro, comprising contacting a target cell with a SIRPα positive phagocytic cell sample in the presence of the antibody or an antigen-binding fragment thereof of claim 1 and/or a pharmaceutical composition comprising the antibody or antigen-binding fragment thereof and one or more pharmaceutically acceptable carriers, thereby inducing the phagocytosis of the target cell by the SIRPα positive phagocytic cell.

63. (canceled)
